(19) United States
(12) Patent Application Publication
(10) Pub. No.: US 2018/0344215 A1
(43) Pub. Date: Dec. 6, 2018

(54) AUTOMATED HEALTH DATA ACQUISITION, PROCESSING AND COMMUNICATION SYSTEM AND METHOD

(71) Applicant: dacadoo ag, Zurich (CH)

(72) Inventors: Peter Ohnemus, Herrliberg (CH); Andre Naef, Zurich (CH); Laurence Jacobs, Thalwil (CH); David Leason, Chappaqua, NY (US)

(21) Appl. No.: 16/038,058

(22) Filed: Jul. 17, 2018

Related U.S. Application Data
(63) Continuation-in-part of application No. 15/778,999, filed on May 24, 2018.
(60) Provisional application No. 62/533,557, filed on Jul. 17, 2017.
(60) Provisional application No. 62/259,593, filed on Nov. 24, 2015.
(60) Provisional application No. 62/269,808, filed on Dec. 18, 2015.
(60) Provisional application No. 62/341,421, filed on May 25, 2016.
(60) Provisional application No. 62/383,027, filed on Sep. 2, 2016.
(60) Provisional application No. 62/409,329, filed on Oct. 17, 2016.

(51) Int. Cl. A61B 5/11; G16H 20/30; G09B 19/00; A63F 13/816; A61B 5/00

(52) U.S. Cl. CPC A61B 5/1118; G16H 20/30; G09B 19/00; A63F 13/816; A61B 5/746; A63F 2300/8005; A61B 5/4866; A61B 5/0002; A61B 5/7475; A61B 5/7435; A61B 5/743

(57) ABSTRACT

Dynamic tracking of user-based activity is provided as a function of information received from a plurality of data sources. Tracking of user activity information via a first application occurs correlated to steps taken by a user over a period of time. A health score associated with the user is presented in a GUI. Further, communication with a second application occurs during gameplay. Moreover, the at least one computing device is configured to combine, using the first application, the tracked user activity information with at least a portion of the user interaction information into an activity value. The computing device updates the health score using the activity value. Still further, the computing device is configured to store the updated health score in association with a record concerning the user, and to cause a notification on the mobile computing device of the updated health score when the first application is not executing.

### CROSS-REFERENCE TO RELATED PATENT APPLICATIONS

This application is based on and claims priority to U.S. Provisional Patent Application 62/533,557, filed Jul. 17, 2017, and further this application is a continuation-in-part of U.S. Non-Provisional patent application Ser. No. 15/778,999, filed May 24, 2018. U.S. Non-Provisional patent application Ser. No. 15/778,999 is a U.S. National Phase Application under 35 U.S.C. § 371 of International Patent Application No. PCT/US2016/063606, filed Nov. 23, 2016, which is based on and claims priority to: U.S. Provisional Patent Application No. 62/259,593, filed Nov. 24, 2015; U.S. Provisional Patent Application No. 62/269,808, filed Dec. 18, 2015; U.S. Provisional Patent Application No. 62/341,421, filed on May 25, 2016; U.S. Provisional Patent Application No. 62/383,027, filed on Sep. 2, 2016; and U.S. Provisional Patent Application No. 62/409,329, filed on Oct. 17, 2016. Each of all the above-identified applications is incorporated by reference as if set forth in its respective entirety herein.

### FIELD

The present application relates, generally, to automated health data acquisition and, more particularly, to automatically classifying health-related activity information in view of biological and/or behavioral tracking and/or external conditions.

### BACKGROUND OF THE INVENTION

Despite advances in many areas of technology, there are still barriers to assessing the relative health of a person in a rapid, cost effective, and timely manner. With the increase in health care costs and prevalence of diseases related to unhealthy lifestyles such as diabetes and heart disease, it is important to assess the relative health of individuals, and this has not been adequately addressed. Moreover, in many areas of the world access to doctors is limited. Even in the developed world, a doctor's time is considered a precious commodity and there are often long waiting lists and doctor-to-specialist referral systems that have to be navigated before being seen. Accordingly, it can be very difficult to gain access to medical professionals in order to receive information about one's health.

Over recent years, fitness tracking devices have been employed to monitor the health and activity of individuals. Devices for biological and physiological monitoring, such as for blood pressure, heartrate and temperature, can communicate via Bluetooth or other wireless protocol to a computing device. Behavioral and activity monitoring can also be provided via one or more devices, such as pedometers and other accelerometer-based devices, which can also send signals representing fitness activity of an individual wirelessly to one or more computing devices. Moreover, hardware has been integrated and implemented in communication devices, such as smart phones, and used in tracking user activity, as well as biological and physiological information. Unfortunately, such tracking devices operate in an isolated fashion and do not collectively contribute to provide an accurate and timely representation of a person's biological, physiological and/or activity-based conditions.

More recently, mobile apps have been developed that integrate signals received from various fitness tracking devices. Exercise goals can be defined by users of such apps, and as a user exercises, the user can be informed of how well he or she is doing to meet such goals. For example, active diaries (e.g., provided by “Moves” app) can record walking, cycling, running and other activity, and also present reports for users regarding distance, duration, number of steps taken and number of calories burned for respective activities.

Despite recent advances in fitness tracking technology, fitness activity continues to be represented in misleading ways and requires user input and manual intervention to correct for inaccuracy.

### SUMMARY

It is with respect to these and other considerations that the present application is presented.

In one or more implementations, the present application provides a system and/or method for dynamic tracking of user-based activity as a function of information received from a plurality of data sources. At least one computing device is configured with a processor and non-transitory processor readable media and instructions that, when executed by the at least one computing device, configures the computing device to track via a first application user activity information correlated to steps taken by a user over a period of time, the first application executing on a mobile computing device and including a graphical user interface that presents at least a health score associated with the user. Further, the at least one computing device is configured to communicate with a second application executed by the mobile computing device gameplay information, the communications including information concerning user interaction with a gaming environment provided by the mobile computing device. Moreover, the at least one computing device is configured to combine, using the first application, the tracked user activity information with at least a portion of the user interaction information into an activity value. Still further, the at least one computing device is configured to update the health score of the user using the activity value. Still further, the at least one computing device is configured to store the updated health score in association with a record concerning the user, and to cause a notification on the mobile computing device of the updated health score when the first application is not executing.

In one or more implementations, the present application provides a system and/or method for updating a health score of a user through dynamic tracking of user-based activity as a function of information received from a plurality of data sources. For example, tracking occurs via a first application user activity information correlated to steps taken by the user over a period of time, the first application executing on a mobile computing device and including a graphical user interface that presents at least the health score. Further, communication occurs with a second application executed by the mobile computing device gameplay information, the communications including information concerning user interaction with a gaming environment provided by the mobile computing device. Still further, using the first application, the tracked user activity information is combined with at least a portion of the user interaction information into an activity value, and the health score of the user is updated using the activity value and stored in association with a record concerning the user. Thereafter, the updated health score is presented in the graphical user interface by causing a notification independent of launching the application.

### BRIEF DESCRIPTION OF THE DRAWING FIGURES

Various features, aspects and advantages of the invention can be appreciated from the following detailed description and the accompanying drawing figures.

FIG. 1 a diagram of an example hardware arrangement in accordance with an example implementation;

FIG. 2 illustrates functional elements of an example information processor and/or workstation in accordance with an implementation of the present application;

FIG. 3 is a block diagram illustrating components associated with a self-learning activity identification system;

FIG. 4 illustrates an example flow of information and steps associated with a self-learning activity identification system in accordance with one or more implementations of the application;

FIG. 5 is an example display screen that illustrates a determination of user activity as a function of a user's heartrate, in accordance with an example implementation of the present application;

FIGS. 6 and 7 illustrate example data entry display screens associated with example implementation of the present application;

FIG. 8 illustrates an example interactive data entry display screen associated with entering data, associated with activity;

FIG. 9 is an example graph that identifies opportunities that can be provided in accordance with one or more implementations of the present application;

FIG. 10 illustrates an example chart identifying populations suitable for servicing in accordance with the present application;

FIG. 11 illustrates example graphs showing traditional and improved life insurance in accordance with one or more implementations of the present application;

FIG. 12 illustrates an example chart that identifies premium percentage discounts offered for users in accordance with the present application;

FIG. 13 illustrates an example display screen that is provided on a mobile computing device and that identifies a respective user's health score at the present time;

FIG. 14 illustrates an example data entry display form that includes options for a user to select in connection with maintaining control over information and privacy;

FIGS. 15-23 identify one or more implementations for engaging a user for information, calculating a health score range and using the information publicly;

FIG. 24 is a block diagram that illustrates the source of example validated records that are suitable for entry in a distributed secure ledger, such as a blockchain;

FIG. 25 is a block diagram illustrating an example blockchain that includes validated private electronic medical records and validated institutional electronic medical records;

FIG. 26 is a block diagram illustrating an example blockchain that includes a smart contract offer;

FIG. 27 illustrates an example network of interaction between counterparties and including distributed ledger's that include smart contract offers from individual owners of health information;

FIG. 28 illustrates an example decentralized marketplace, including distributed ledgers of life in accordance with an example implementation of the present application;

FIG. 29 illustrates an alternative representation of the present application including health information that is generated in distributed as a function of health devices;

FIG. 30 illustrates example features and benefits of the present application which use of a blockchain or similar technology serves for transforming various industry, such as insurance and other health related industries;

FIG. 31 illustrates a flow diagram of one example implementation of the present application in accordance with exchange;

FIG. 32 illustrates a flow diagram of an example alternative implementation of the present application in accordance with at least a decentralized smart contract model;

FIG. 33 is a diagram illustrating dynamic activity data integration and data flow in accordance with an example implementation;

FIG. 34 is a flow chart illustrating steps associated with an example implementation of the present application;

FIG. 35 is a block diagram that illustrates example modules executed by a processor in an example implementation;

FIG. 36 illustrates graphs representing features associated with a walk score, in accordance with the present application;

FIG. 37 illustrates an example walk score value a corresponding number of days until the walk score decays or drops, as well as a financial representation associated with a health reservoir;

FIG. 38 illustrates an example implementation that includes a graphical virtual mapping functionality, in accordance with the present application;

FIGS. 39-44 illustrate example screen displays of a game platform in accordance with an example implementation of the present application;

FIGS. 45A and 45B illustrate example display screens that display respective gameplay levels that the user has reached and/or unlocked;

FIGS. 46-49 illustrate example display screens that provide information associated with activity, accomplishments, and status over time, in accordance with one or more implementations of the present application;

FIGS. 50 and 51 illustrate an example implementation of a photo Safari, in connection with the present application;

FIG. 52 illustrates an example screen display showing an on-line reward shop, in which users can redeem coins for goods/services in accordance with one or more implementations of the present application;

FIG. 53 illustrates an example data screen display of a data record representing a settled spawn, in accordance with the present application;

FIG. 54 illustrates an example image representing an item with a gem overlaid thereon, in accordance with an example implementation of the present application; and

FIG. 55 illustrates an example display screen 5500 for defining respective user settings, in accordance with an example implementation of the present application.

### DETAILED DESCRIPTION OF THE EMBODIMENTS

By way of overview and introduction, the present application provides an improved system and method for integrating information received from a plurality of distinct tracking devices with historical and behavioral information associated with user activity and health. A sophisticated, machine-learning health and activity classification engine is provided that analyzes information received from various sources, including fitness tracking devices, to classify a user's activity and health. Information representing individual activity units can be flexibly joined together to represent specific activity in a new and accurate way. Moreover, the present application provides a predictive element that, beyond simply determining specific activity that a user may be engaged in, defines activity goals that are modified over time to encourage and press users to adapt to more strenuous (or less strenuous) exercise regimen.

In one or more implementations, a classification engine is provided with a sequential and/or continuous stream of user information that is passively tracked. Such information can include sensor data from various sources, such as a pulse monitoring device, one or more accelerometer-related applications (e.g., a pedometer or other motion detecting application), a temperature sensing device, a pulse oximeter and/or a global positioning system (“GPS”) device. In addition to such sensor data, user profile information is accessed and/or managed that represents, for example, a user's age, gender, height and weight, as well as various biological and/or physiological characteristics that are particular to an individual. One more modules can be configured by executing code to aggregate such user profile information. Results of one or more aggregation processes can provide demographic information, which can function as input to other processes, such as predictive mechanisms in connection with people's health and/or activity. Moreover, other sources of information can be accessed or otherwise managed by the classification engine that contribute to determining and/or predicting various forms of user activity and health. For example, calendar information for a user's personal and/or professional schedule can be accessed from one or more computing devices, such as mobile computing devices, and managed by the classification engine. Such calendar information can include workout schedules, personal schedules, work schedules or virtually any other calendar entry. The classification engine can further be configured to access and process various external data sets, such as time of day, as well as specific day, week and month information, including local weather, in order to classify and predict user activity and health.

Thus, information from various sources, including sensors, user profile databases and calendar-related information can be received, managed and processed. One or more processors can execute code, such as to provide respective modules, and process the information to recognize activity patterns and predict and classify specific activity that a person may be engaged in. As a person engages in a respective activity, such as one or more of a plurality of regular workouts, a determination module can assess that the user has reached a plateau and should be engaged in more strenuous and/or vigorous activities. As a person physically develops, such as a result of engaging in various and regular exercise, that person's tolerance improves and the effectiveness of the initial exercise regimen reduces. Thus, one aspect of the classification engine is to access information associated with a user's workout and detect when the person is ready to advance further in his or her exercise regimen. Thereafter, information can be used to coach the user accordingly, such as in connection via one or more interactive graphical user interfaces provided by a coaching module.

In one or more implementations of the present application, a user's heartrate can be periodically or continuously detected and monitored. A baseline heartrate and/or a calculated MET value, can be established as a function of such monitoring, and any detected heartrate within a respective range (e.g., between 65 and 75 bpm) can be used to form a determination that a user is at rest. At another point in time when the user's heartrate is elevated, such as to 105 beats per minute (“bmp”), a determination can be made that the user is performing some activity. Upon detection of the change in heartrate, one or more algorithms can be executed by a computing device and the respective activity can be automatically determined and classified, in accordance with the teachings herein, to define an activity unit having a start time, an end time, and an activity name. In other implementations, activity units can be defined based on other information sources provided to the classification engine, such as location information.

### Hardware/Software Modules and Implementation

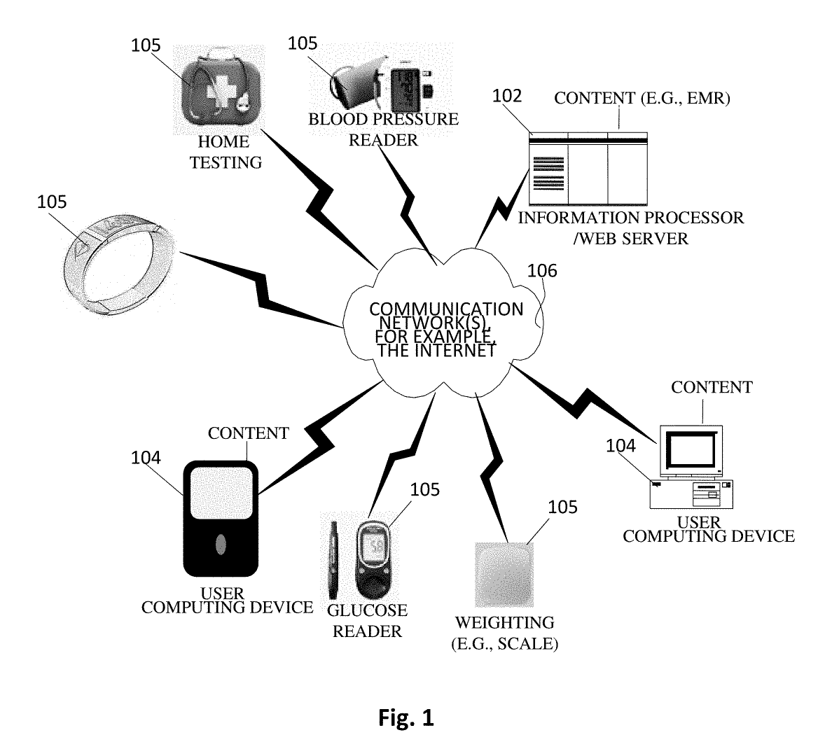

Referring now to the drawing figures, in which like reference numerals refer to like elements, FIG. 1 shows an example hardware arrangement for obtaining, processing, and outputting content, including via a communication network, such as the Internet. As shown in FIG. 1, one or more of a plurality of user computing devices, such as information processor(s) 102 and user computing devices(s) 104 are configured to receive and/or send electronic content from/to one or more health devices 105. In the example illustrated in FIG. 1, such health devices 105 include, but are not limited to, medical home testing kit 105, blood pressure reader 105, wearable band 105, glucose reader 105 and weighting reader (scale) 105. Thus, as illustrated in FIG. 1, the present application provides a decentralized system in which data acquisition, data storage, and data processing can occur as a function of independently operating and configurable devices.

In one or more implementations, information processed in accordance with the present application can be used to produce a numerical score as a basis for assessing the relative health of a user (i.e., production of a user's health score). Specific implementations regarding features associated with calculating and distributing a user's health score is more fully shown and described in commonly assigned U.S. Non-Provisional patent application Ser. No. 14/257,855, entitled “AUTOMATED HEALTH DATA ACQUISITION, PROCESSING AND COMMUNICATION SYSTEM,” which is incorporated by reference in its entirety herein.

The present application supports computer-based systems and methods for the collection of health-related parameters of a user and a user interface for the presentation (e.g., display) of data, as a function of one or more applications executing on one or more user computing devices 104 and/or information processors 102. The computer-based application can be implemented via a microcontroller that includes a processor, a memory and code executing therein so as to configure the processor to perform at least some of the functionality described herein. The memory can store data and instructions suitable for controlling the operation of one or more processors. An implementation of memory can include, by way of example and not limitation, a random access memory (RAM), a hard drive, or a read only memory (ROM). One of the components stored in the memory is a program. Specific instances regarding validating, storing, and transacting in accordance with secured ledgers associated with a user's health information (including health score information) are described in greater detail below.

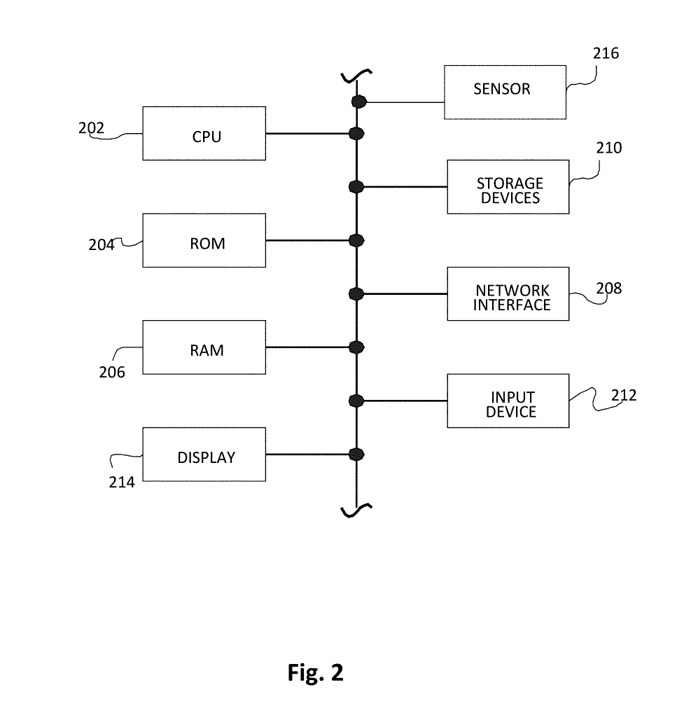

FIG. 2 illustrates one or more functional elements associated with information processor 102, user computing device 104 and/or health device(s) 105, and include a processing subsystem having one or more central processing units (CPU) 202 used to execute software code and control the operation of device 102, 104 and/or 105, and processor-readable media including, for example, read-only memory (ROM) 204, random access memory (RAM) 206, as well as one or more network interfaces 208 to transmit and receive data to and from other computing devices across a communication network, storage 210 such as a solid state memory, a hard disk drive, CD ROM or DVD ROM for storing program code, databases and application data, one or more input devices 212 such as a keyboard, mouse, track ball, virtual keyboard, touchscreen, microphone and the like, and a display 214.

The memory 204 and/or 206 can be a persistent or non-persistent storage device that is operative to store an operating system for the processor in addition to one or more of software modules. In accordance with one or more embodiments, the memory comprises one or more volatile and non-volatile memories, such as Read Only Memory (“ROM”), Random Access Memory (“RAM”), Electrically Erasable Programmable Read-Only Memory (“EEPROM”), Phase Change Memory (“PCM”), Single In-line Memory (“SIMM”), Dual In-line Memory (“DIMM”) or other memory types. Such memories can be fixed or removable, as is known to those of ordinary skill in the art, such as through the use of removable media cards or modules. In addition, one or more remote or local storage devices, such as databases, may comprise caches, including database caches and/or web caches. Programmatically, data storage may comprise flat-file data store, a relational database, an object-oriented database, a hybrid relational-object database, a key-value data store such as HADOOP or MONGODB, in addition to other systems for the structure and retrieval of data that are well known to those of skill in the art.

As used herein, the term, “processor” or “computer” refers, generally, to one or more electronic devices (e.g. semiconductor-based microcontrollers) configured with code in the form of software, to execute a given instruction set. For example, the mobile computing device 104 can include one or more processing or computing elements executing commercially available or custom operating system, e.g., MICROSOFT WINDOWS, APPLE OSX, UNIX or Linux based operating system implementations, such as APPLE IPAD/IPHONES®, ANDROID® devices or other electronic devices. In other implementations, the mobile computing device 104 may be one of any custom or non-standard hardware, firmware or software configurations. The mobile computing device 104 can communicate with the one or more remote networks using USB, digital input/output pins, eSATA, parallel ports, serial ports, FIREWIRE, Wi-Fi, Bluetooth, RF transmitters/transponders or other communication interfaces. In a particular configuration, the mobile computing device(s) are also configured, through hardware and software modules, to connect to more remote servers, computers, peripherals or other hardware using standard or custom communication protocols and settings (e.g., TCP/IP, etc.) either through a local or remote network or through the Internet 106.

Moreover, the information processor 102 referenced herein can include servers, cloud computing platforms, micro-computing elements, computer(s)-on-chip, home entertainment consoles, media players, set-top boxes, prototyping devices or “hobby” computing elements. Such computing elements described are connected, directly or indirectly, to one or more memory storage devices (memories) to form a microcontroller structure.

The computer memories may also comprise secondary computer memory, such as magnetic or optical disk drives or flash memory, that provide long term storage of data in a manner similar to the persistent memory device. In one or more embodiments, the memory of the processors provide for storage of application programs and data files when needed.

The processors or computers described are configured to execute code written in a standard, custom, proprietary or modified programming language such as a standard set, subset, superset or extended set of JavaScript, PHP, Ruby, Scala, Erlang, C, C++, Objective C, Swift, C#, Java, Assembly, Go, Python, Pearl, R, Visual Basic, Lisp, or Julia or any other object oriented, functional or other paradigm based programming language.

Respective computer programs can include instructions that cause the processor 202 to execute steps that implement the methods described herein. The program can be implemented as a single module or as a plurality of modules that operate in cooperation with one another. The program can include software that can be used in connection with one or more implementations of the present application. For example, a communication subsystem can be provided for communicating information from the microprocessor to the user interface, such as an external device (e.g., handheld unit or a computer that is connected over a network to the communication subsystem). Information can be communicated by the communication subsystem in a variety of ways including Bluetooth, Wi-Fi, Wi-Max, RF transmission, near-field communications or other suitable communication protocol. A number of different network topologies can be utilized in a conventional manner, such as wired, optical, 3G, 4G, 5G or other suitable networking protocol.

The communication subsystem can be part of a communicative electronic device including, by way of example, a smart phone or cellular telephone, a personal digital assistant (PDA), tablet computer, netbook, laptop computer, or other computing device. For instance, the communication subsystem can be directly connected through a device such as a smartphone such as an iPhone, Google Android Phone, Samsung Tizen, BlackBerry, and Microsoft Windows Mobile enabled phone, or a device such as a heartrate or blood pressure monitor, weight measurement scales, exercise equipment or the like. One or more of these devices can include or otherwise interface with a module or communication unit with the subsystem to allow information and control signals to flow between the subsystem and the external user interface device. The communication sub-system can cooperate with a conventional communicative device or can be part of a device that is dedicated to the purpose of communicating information processed by the microcontroller.

Content provided in accordance with devices 102, 104 and/or 105 can include, for example, numerical, textual, graphical, pictorial, audio and video material. Communication of such content can occur by and between one or more of the respective devices 102, 104 and 105. Thus, in one or more implementations, any of the health devices 105 can employ hardware and software modules that collect and/or receive information, process information, and transmit information to device 102/104. Information processor 102 and/or user computing device 104 can employ software for enabling a communication session, for example, an HTTP session, to be established between the information processor 102/user workstation 104 and one or more various respective devices, including to effect secure blockchain transactions.

In addition to monitoring biological and physiological characteristics of a user, health devices 105 can be configured to monitor exercise, such as steps that are identified by interval, size and/or by intensity. A number of steps taken in quick succession, for example, may be more strenuous than taken over a long period of time. Moreover, uphill steps may be more strenuous than downhill steps. These and other factors may be included with the exercise/activity data from respective health device 105. Additionally, a respective health device 105 can be configured with global positioning system (“GPS”) technology, which may provide location information regarding altitude, steepness of steps, or other relevant geographic information. In another example, one or more of the health devices 105 can be configured with a galvanic skin sensor, for example, to measure stress levels of the wearer. Moreover, a health device 105 can be configured for sleep monitoring of the wearer as well. In one or more implementations, the health band 101 is configured with memory for storing information associated the wearer. In addition to obtaining and/or generating information substantially automatically, information regarding exercise and/or other activity may be input by the wearer, including via a user interface via BLUETOOTH or other suitable communications with the user computing device 104.

In addition to receiving and transmitting information associated with the wearer, one or more of the health devices 105 can be configured to process information, such as in connection with information collected and/or processed by sensing components. “Raw” data, which may be in the form of signals, may be collected by a respective health device 105 and processed to represent biological and/or physiological information. Such information can include personal identification information, blood type, DNA information, weight information, blood pressure information, and other medical, physiological and/or biological information. A respective health device 105 can be further configured with one or more algorithms stored in a memory which, when executed by a processor, calculates information, such as the wearer's health score. The wearer's health score (and/or the processed biological/physiological information) can be, thereafter, transmitted to the information processor 102/user computing device 104 for further processing, such as establishing secure private objects for storage and validation in a validated ledger record (e.g., a blockchain), or other suitable technology.

### Self-Learning Activity Identification

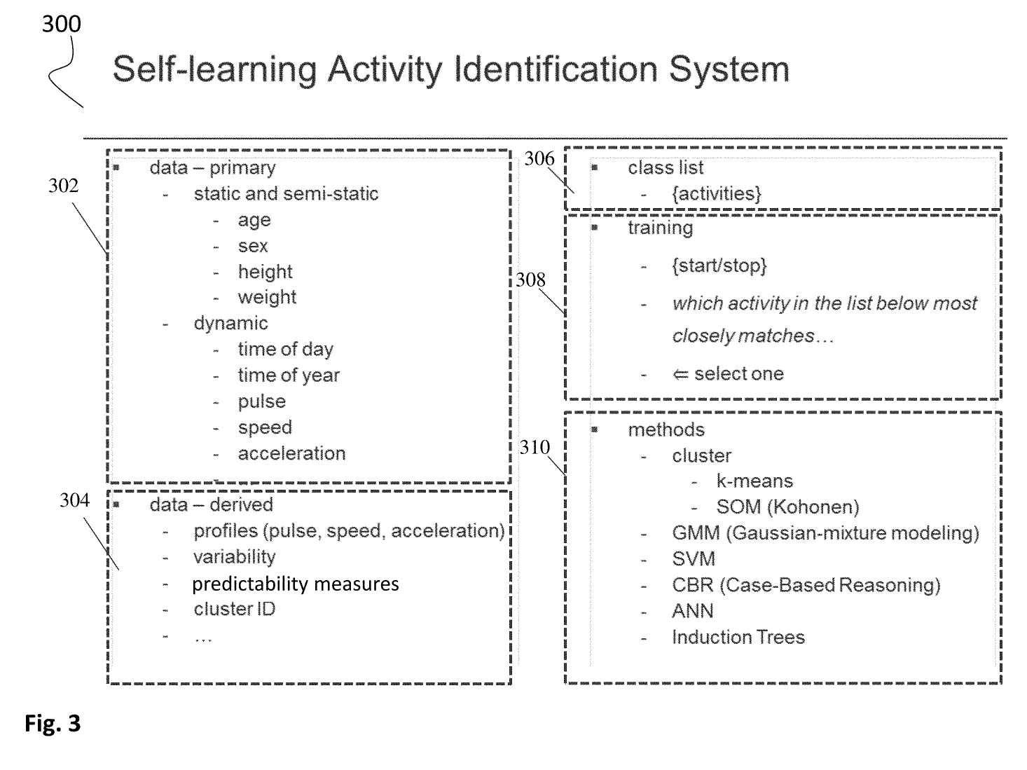

Referring now to the drawings figures in which like reference numerals refer to like elements, FIG. 3 is a block diagram illustrating components associated with a self-learning activity identification system 300, in accordance with an implementation of the present application. As illustrated in FIG. 3, primary data 302 can include, for example, static and semi-static values associated with a user's age, gender, height and weight. Dynamic primary data can include dynamic values that are sensed or detected, such as time of day, time of year, heartrate (pulse) speed and acceleration. Derived data values 304 can include profiles associated with heartrate, speed and acceleration, variability, predictability measures and cluster ID. Activity class list 306 represents, for example, activities that a user can be engaged in and is usable by the classification engine to determine and classify activity that is detected as a function of biological and/or physiological values, e.g., pulse rate, temperature, blood pressure or the like. Examples of user activities can include, for example, running, walking, swimming or engaging in various sports. In one or more implementations of the present application, the classification engine can be trained, such as for future classification of activity. For example, training section 308 identifies starting and stopping training operations, and enabling selectable options for users to identify respective activity that may have occurred or be occurring. Machine learning methods 310 can include cluster-based learning, such as for pattern recognition (e.g., K-Mean clustering, self-organizing mapping (SOM, Kohonen), Gaussian-Mixture Modeling (GMM), Support Vector Machine (SVM), Case-Based Reasoning (CBR), ANN, and Induction Trees).

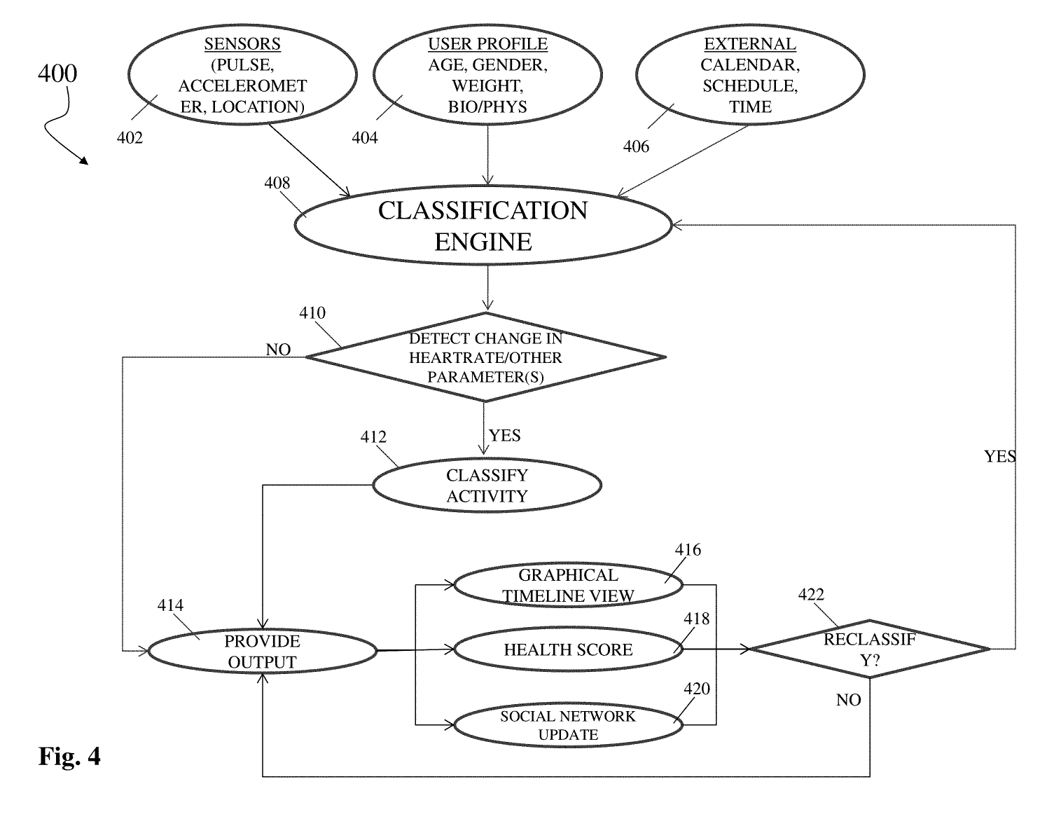

FIG. 4 illustrates an example flow of information and steps associated with a self-learning activity identification system in accordance with one or more implementations of the application. Sensor information 402, user profile information 404 and external information 406 can be transmitted to or otherwise accessed by classification engine 408. At step 410, a change is detected, for example, in the user's heartrate, respiratory rate or other parameter. As noted above, a user's heartrate while at rest can be established as a parameter, and as sensed information is obtained by one or more processors that the user's heartrate has exceeded the at rest parameter, a determination can be made that some user activity, such as exercise, is occurring. Thereafter, the activity is classified as specific activity, such as a function of one or more machine learning methods 310 (412), with a start time and, eventually, after detection of the conclusion of the activity, an end time. It should be appreciated that when one activity ends, another, in theory, begins, if only an “at rest” activity. Whether the activity conclusion and start have been correctly discerned by the algorithm or established is a facet of one or more implementations of the present application.

The step of classifying activity is not trivial and can be the result of complex processing of dynamic information received from a plurality of sensing devices with profile and external data that are accessible to one or more computing devices. For example, using GPS and calendar information, one or more machine learning techniques detect a pattern that a user goes to the gym between the hours of 4:00-5:00 p.m. on Sundays. The user may also provide information via a feedback loop (shown and described herein) that the user enjoys body pump (weightlifting) activity most, and is engaged in body pump challenges with other users. Upon a detection of a change in heartrate from the baseline condition, available signals from tracking devices now known or later developed can be processed to classify the respective activity that the user is engaged in. In another example, user profile information may represent that a user engages in skiing activity and sailing activity. Using various forms of dynamic and external information, detected activity can be classified correctly. For example, a computing device would be incorrectly classifying user activity as sailing in view of GPS coordinates that identify that the user located on top of a mountain, and in view of calendar information that represents that it is wintertime, and further in view of weather information that represents it is snowing where the user is located. Such activity, in view of all of the relevant composited information and associated with the user's elevated heartrate would correctly classify the activity as skiing. Moreover, monitoring heartrate information (and other physiological and metabolic-based information) enables a period of time for which activity is occurring.

Thus, the present application provides sophisticated processing of activity-based information to classify an activity accurately. This can be the result of processing specific and discrete periods of time and various information shown and described herein. For example, discrete determinations represent that a user is very active for five minutes, relatively inactive for the next two minutes, and active again for the following five minutes could result in three activity units being defined, or three minor units of a major activity unit; each having respective begin and end times. Location-based information over the course of the same 12 minutes represents that the user traveled five miles. An inaccurate classification of activity would be that the user walked briskly to a bus stop, rode a bus for 4-5 miles, got off the bus and continued walking briskly. However, the present application correctly classifies the user's activity as riding a bicycle and the periods of strenuous activity represent peddling uphill, the period of relative inactivity represents coasting downhill. The automated classification engine in accordance with the present application analyzes not just one or two data points but provides for a holistic and complex processing of many sources of information, including extrinsic activity such as time of day, day of week, location and weather, to correctly classify activity. For example, the same 12 minutes activity information that are identified to have occurred late at night and in the winter during a heavy snowfall would not result in a classification of bicycle riding. This represents the complex arrangements and collaborative-based system of respective hardware and software devices that contribute to an analysis of discrete portions of activity over time for improved and accurate classification of such activity.

In addition to providing automatic classifications of user activity, the present application provides for fault tolerance and provides users with an ability to reclassify activity. In one or more implementations, a user interface is provided that includes interactive and selectable elements that represent user activity over time (e.g., a graphical representation of a timeline). For example, a timeline is generated and displayed that identifies when a user woke, a period of time, distance and number of steps taken before getting in a car, the distance and time spent while in motorized transit, the distance, period of time, and number of steps while running, a period of time in which the user is thereafter dormant (e.g., sitting behind a desk), the amount of food consumed during lunch, a period of time, distance and number of steps taken during an afternoon run, a period of time in which the user is again dormant (e.g., sitting behind the desk), and a period of time and type of activity after the user left work (e.g., 15 minutes of bicycle riding for five miles). In one or more implementations, each of the respective timeline entries comprises a flexible activity unit which is selectable, and the user can take corrective action, such as to reclassify the activity, merge several activity units, revise the amount of time during the activity or revise any other data element associated therewith. Each time a reclassification or other revision is made, such information is preferably provided back to the classification engine for further learning. Optionally, changes to activity unit labels, duration, and joinder to other activity units can be included in an audit trail for reporting and compliance review purposes.

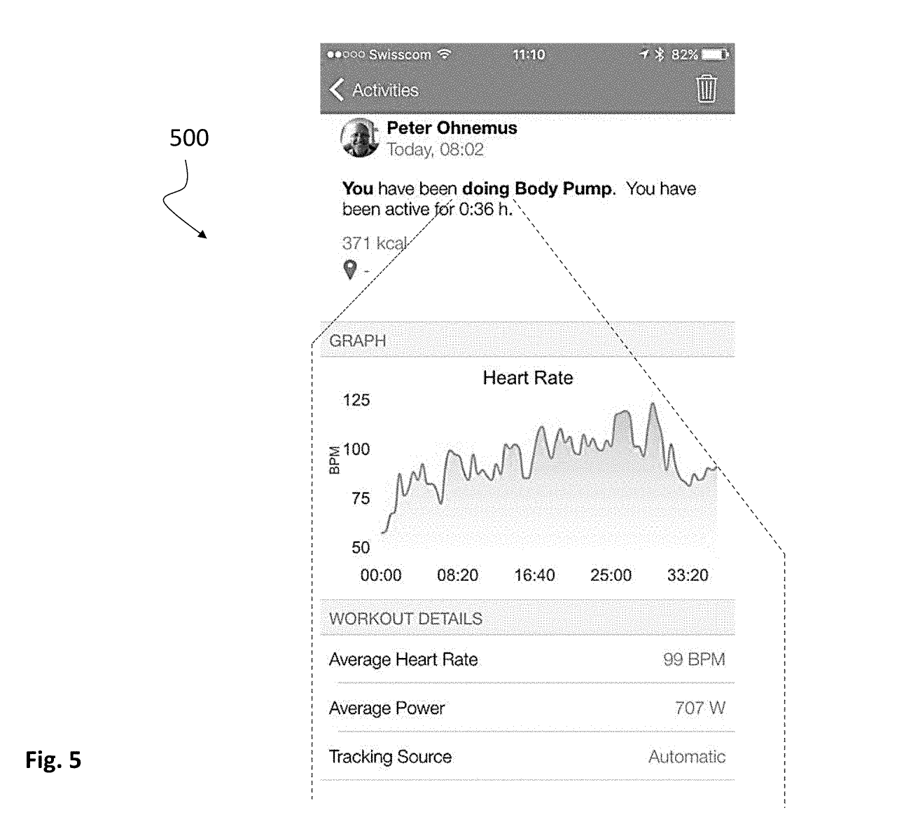

FIG. 5 illustrates an example display screen 500, such as provided on a mobile computing device (e.g., a smartphone), in which user activity has be detected substantially automatically, such as by monitoring heartrate and during a specific period of time (e.g., 0.36 h). The user's activity can be determined, for example, via machine learning in accordance with the teachings herein. The user can further, for example, be prompted to communicate with the avatar to express how the user felt during the respective activity. Other information illustrated in FIG. 5 includes a graphical display of the user's heartrate over time, as well as determinations of average heartrate, average power, and tracking source.

Continuing with reference to the process/information flow 400, at step 414 output is provided, for example, to a computing device associated with a user, that represents the classified activity. For example, a graphical representation of timeline is generated and provided (416), a user's health score (as shown and described herein) is generated and provided (418), an update is provided to one or more social networks (420) or other suitable output is provided. Thereafter, a determination is made that activity information, such as provided in a timeline, is to be reclassified (422). If so, then the process branches to 408 and information associated with the reclassified entry is provided back to the classification engine. Alternatively, if the determination in 422 is that no reclassification is needed, then the process branches back to step 414 and output is provided.

In one or more implementations, individual activity units representing discrete periods of activity are classified automatically by a computing device in accordance with information processed by the health and activity classification engine. For example, a user at a gym spends 10 minutes in body pump activity and raises his heartrate to 110 bpm. After a few moments, the user's heartrate returns to an at-rest state (e.g., 70 bpm). In this example, the determination that the heartrate has resumed an at-rest state signifies the end of the discrete activity unit. Thereafter, the user spends 5 minutes on a treadmill, and raises his heartrate again to 105 bpm, thereby signifying to the computing device another discrete activity unit. Thereafter, the user spends 5 minutes on a stationary bicycle, which is interpreted as a third discrete activity unit. Each of these activity units can be joined into a major activity unit, e.g., “gym workout” which has an overall duration that spans the beginning of the body pump activity through the end of the treadmill activity. This joinder is different than a merge, which is an alternative way of reducing the number of activity units by combining two or more units.

The benefit of the present application is that activity units are provided in a flexible format that enable users to reclassify activity units, as well as to combine a plurality of activity units into a unified group, such as a plurality of “minor” activity units into a single “major” activity unit or merge two or more units into a single major or minor unit. Continuing with the previous example, the user reviews a timeline of activity that occurred while the user was at the gym. Upon detecting three respective activity units (body pump, treadmill and stationary bicycle) the user makes selections, via one or more graphical screen controls provided in the user interface, to classify the plurality of activity units into a single activity unit: “gym workout.” Thus, minor activity units can be classified as part of a single major activity unit, in accordance with the present application. Similarly, options can be provided for user to parse a single activity unit into a plurality of activity units. As activity is reclassified, including into single (major) or multiple (minor) units, information is provided back to the classification engine and used for future classifying of user activity, as well as for audit purposes.

While many of the descriptions set forth in here identify changes in a user's heartrate to trigger a determination of specific user activity, the application is not so limited. As described herein, the classification engine is configured to access or otherwise receive information from sensors, individuals as well as external sources. Any one or combination of information sources can be used as a triggering by the classification engine for detecting and/or classifying respective user activity. For example, information from a GPS device and calendar source can be processed to determine that a user has arrived at a gym at a certain time of day and on a certain day of the week. This combination of data points can be used to demarcate a major activity unit: gym workout. While the user engages in various exercise activities, such as body pumping, stationary bicycling and running on a treadmill, the classification engine can define minor activity units associated with each and can generate and provide one or more summaries representing specific activity, the duration of such activity, a graphical representation of the user's heartrate during such activity over the duration, or the like. Thus, the classification engine can be used to classify broadly a plurality of activities into a single major activity unit, while maintaining and providing information on each of the respective minor activities comprising the major unit.

In one or more example implementations, the present application provides a computer-implemented system and method configured to acquire health-related and/or medical-related data, and to process the data. For example, the systems and methods herein provide for providing feedback substantially in real-time via an on-line and/or mobile platform. Using the systems and methods disclosed herein, information can be received from user devices, and the information can be processed to provide various form of feedback, such as alerts and notifications. Upon accessing one or more modules associated with the present application, such as by a user submitting initial registration information, a baseline assessment in the form of a health score can be calculated and provided to a user at no or nominal charge. This results in engaging users with valuable health-related information and increases the likelihood that users will remain engaged and regularly provide health-related information that can be used in connection, for example, with a metric health model score and/or a quality of life model score.

In one or more implementations, the baseline assessment (e.g., the health score) is based on four data points: Age, Height, Gender and Weight (ex. Instant Health Score). In one or more implementations, a health score distribution of an organization, such as based on exciting data. A health score can be calculated across an enterprise, such as in a stateless manner, that precludes social networking, gamification or other individual identifying element.

The present application provides a distributed system in which data acquisition, data storage, and data processing can be used to produce a numerical score as a basis for assessing the relative health of a user. In an implementation, a computer-based application is provided for the collection of health-related parameters of a user and a user interface for the presentation (e.g., display) of data. The computer-based application can be implemented via a microcontroller that includes a processor, a memory and code executing therein so as to configure the processor to perform at least some of the functionality described herein. The memory can store data and instructions suitable for controlling the operation of one or more processors. An implementation of memory can include, by way of example and not limitation, a random access memory (RAM), a hard drive, or a read only memory (ROM). One of the components stored in the memory is a program. The program includes instructions that cause the processor to execute steps that implement the methods described herein. The program can be implemented as a single module or as a plurality of modules that operate in cooperation with one another. The program can include software that can be used in connection with one or more implementations of the present application.

A communication subsystem can be provided for communicating information from the microprocessor to the user interface, such as an external device (e.g., handheld unit or a computer that is connected over a network to the communication subsystem). Information can be communicated by the communication subsystem in a variety of ways including Bluetooth, Wi-Fi, Wi-Max, RF transmission, near-field communications or other suitable communication protocol. A number of different network topologies can be utilized in a conventional manner, such as wired, optical, 3G, 4G or other suitable networking protocol.

The communication subsystem can be part of a communicative electronic device including, by way of example, a smart phone or cellular telephone, a personal digital assistant (PDA), tablet computer, netbook, laptop computer, or other computing device. For instance, the communication subsystem can be directly connected through a device such as a smartphone such as an iPhone, Google Android Phone, BlackBerry, and Microsoft Windows Mobile enabled phone, or a device such as a heartrate or blood pressure monitor, weight measurement scales, exercise equipment or the like. One or more of these devices can include or otherwise interface with a module or communication unit with the subsystem to allow information and control signals to flow between the subsystem and the external user interface device. The communication sub-system can cooperate with a conventional communicative device, or can be part of a device that is dedicated to the purpose of communicating information processed by the microcontroller.

It is recognized that users can become confused regarding which respective device should be activated and when. Referred to herein, generally, as “double counting,” users may inadvertently account for the same activity and/or health-related event more than once, which can result in inaccuracy and calculations that distort a user's overall health score. Moreover, some users may provide unclear or partial replies, or even intentionally distort facts. The automated processes and devices shown and described herein reduce or preclude the opportunity for users to accidentally or intentionally distort or misidentify various activity information. Moreover, audit trails can be created, as noted, to assist user's in classification and to enforce compliance.

When a communicative electronic device such as the types noted herein are used as an external user interface device, the display, processor, and memory of such devices can be used to process the health-related information, such as to calculate and provide a numerical assessment. Otherwise, the system can include a display and a memory that are associated with the external device and used to support data communication in real-time or otherwise. More generally, the system includes a user interface, which can be implemented, in part, by software modules executing in the processor of the microcontroller or under control of the external device. In part, the user interface can also include an output device such as a display (e.g., the display). Display may include, for example, organic light emitting diode (OLED) displays, thin film transistor liquid crystal displays and plasma displays.

Moreover, one or more computing devices, including a server, a smartphone, a laptop, tablet or other computing device can send and receive electronic content to/from a health band that is worn by a user. The content may include, for example, numerical, textual, graphical, pictorial, audio and video material. Such communications may occur directly and/or indirectly, such as between the server and the health band via a mobile computing device such as a smart phone, tablet computer or other device. Alternatively, such communications may occur between the server and the health band without the use of computing device. Thus, in one or more implementations, the health band may employ hardware and software modules that collect and/or receive information, process information, and transmit information between the health band and a server and/or between the health band and a mobile device.

In addition to communications-related modules, the respective health devices 105 can be configured with hardware and/or software modules that configure one or more of the health devices 105 to track various forms of biological/physiological characteristics, exercise and/or activity of the wearer. For example, a health device 105 can be configured with one or more sensors and software that collect information relating to biological/physiological characteristics of the wearer, such as temperature, heart rate, heart rate variability (“HRV”), blood pressure, perspiration or the like. Sensors can be used to directly collect health information about a user and transmit that information to user computing device 104 and/or information processor 102. A biosensor can be placed in contact with the user's body to measure vital signs or other health related information from the user. For example, the biosensor can be a pulse meter that is worn by the user in contact with the user's body so that the pulse of the user can be sensed, a heart rate monitor, an electrocardiogram device, a pedometer, a blood glucose monitor or one of many other devices or systems. The biosensor can include a communication module (e.g., communication subsystem) so that the biosensor can communicate, either wired or wirelessly, the sensed data. The biosensor can communicate the sensed data to the user interface device, which in turn communicates that information to the microcontroller. Optionally, the biosensor can directly communicate the sensed the data to the microprocessor. The use of biosensors provides a degree of reliability in the data reported because it eliminates user error associated with manually, self-reported data.

In one or more implementations of the present application, one or more algorithms can be implemented by the server, the mobile device and/or the health band that is based on the user's heartrate. One or more modules can detect automatically the user's average heartrate during the day and convert that into MET per day. For example, the user's heartrate is monitored over time and, thereafter, averaged. Thereafter, while the user's heartrate increases, user activity can be determined and/or detected.

In one or more implementations, the present application provides a health platform, including for calculating a health score, as well as implementing many of the features shown and described herein. The health platform system in accordance with the present application can be accessed via Internet web browser software applications (e.g., CHROME, FIREFOX, SAFARI, INTERNET EXPLORER), and by using a desktop or laptop computer as well as from a mobile device, such as a Smartphone or Tablet via a mobile optimized version of the web site. Moreover, one or more graphical user interfaces provide access for a user, such as via screen controls (e.g., buttons, icons, drop-down lists, radio buttons, checkboxes, textboxes or the like) to enter, view, change and/or delete information. For example, information, such as relating to indoor and outdoor activity can be inserted manually via a web form or via a mobile platform and users can also choose to upload images together the information associated with their activity. Alternatively, (or in addition), data entry can occur substantially automatically, such as via an import process of one or more files formatted in one of various file types (e.g., TXT, DOC, PNG, JPEG, GIF, GPX, and TCX).

The health platform provided in accordance with the present application can be configured with a smartphone software application, referred to herein generally, as a “tracker application,” to track fitness activities in an easy and automatic way (in addition to providing for manual entry) and the recorded/tracked activities can be uploaded automatically on the health platform. The tracker application can be provided for devices operating IOS, Android and BlackBerry operating systems, and can be provided at no charge to the user. For example, workout data can be uploaded via the tracker application.

In one or more implementations, the present application offers the tracker application to track a user's fitness activity, and can be implemented on devices running IOS, ANDROID, WINDOWS PHONE, BLACKBERRY and other suitable mobile device operating systems. Outdoor and indoor activities can be tracked, and data upload to a server computer or other device can be provided in a secure format. The data can be seamlessly and automatically integrated for calculating a user's health score. For example, daily activity measured by step counters/pedometers or other similar devices can be integrated using the systems and methods shown and described herein.

### Passive Tracking and Feedback Loop Modules

In one or more implementations, an interactive interface is provided for interacting with a user, including to encourage the user to provide health-related information. Generally, notifications can include questionnaires or prompts for information, and can be presented by an avatar or virtually any other interactive mechanisms.

As noted above, the present application can be configured to provide representations of “minor” activity units that are classified in a respective “major” activity unit. Moreover, as a user engages in an exercise regimen, one or more activity goals can be defined that are modified over time to encourage and press users to engage in more strenuous activity. In one or more implementations, an avatar or other interactive interface can prompt the user to adjust a major activity unit in an effort to recommend engaging in an exercise activity that may require additional effort or energy. Upon receiving an indication that the user is agreeable, the present application can dynamically adjust the minor activity units, such as the length of time for performing one or more of the activity units, or increasing an adjustable setting associated with particular equipment (e.g., a resistance of a treadmill). Thus, the present application can be configured to monitor and classify respective activity and adjust such activity in one or more ways to increase exercise regimens and improve health.

In addition to a baseline calculated health score, the present application includes one or more modules that provide a form of a health score screener that can provide health score values that go beyond the individual person level. Providing health-related information associated with a population in a concise, anonymous and meaningful format can aid many industries and technologies. For example, insurance companies can use population-based health scores for risk modeling, and can assist employers to reduce their premiums and improve the health of their employees by identifying specific at-risk populations. Populations can be defined in countless ways, including by geography, by socio-economic demographic, by gender and by occupation, to name but a few. Health-related information, including population-based health scores, can identify a return on investment (“ROI”) and associated costs resulting inaction.

In a broad aspect, a method according to the invention can be understood as collecting health related information, processing the information into a health score, and publishing the health score is provided. A system for implementing the method can include a computer having, a processor, memory, and code modules executing in the processor for the collection, processing, and publishing of the information. Baseline information can be determined and provided in response to relatively little and initially received responses from a user, which can engage the user in the future. Further, population health score information can be provided, including as a function of demographics. In addition, changes in heartrate information can be detected, and used to determine (including automatically) respective activity of a user.

In one or more implementations, a daily, substantially real-time calculated health score is provided and is integrated in connection with a feedback loop and artificial intelligence (e.g., avatar) that enables users to learn about their health and lifestyles. The health score can be provided with content in connection with activity tracking, sleep monitoring and/or stress exposure. In connection with activity monitoring, a user's heartrate and/or calculated MET can be based on information detected by breast heart bands, smart watches, soft sensors, contact devices, nano sensors, and/or other suitable technology.

Moreover, one or more goals and/or challenges can be tailored for individuals' lifestyles. The result can be a relevant, easy and fun factor that represents a degree in which a user and/or members of an organization are involved with maintaining good health in accordance with the teachings herein. Moreover, a reporting module can be provided for individuals and/or various groups of persons, such as company departments, profit centers, entire companies, and even regionally (e.g., over one or more countries). The result is significant cost savings attributed to healthcare.

In non-limiting use, a passive tracking device can include a one or more passive tracking devices. Also in non-limiting use, a processor can include one or more processors. Still further in non-limiting use, a database can include one or more databases.

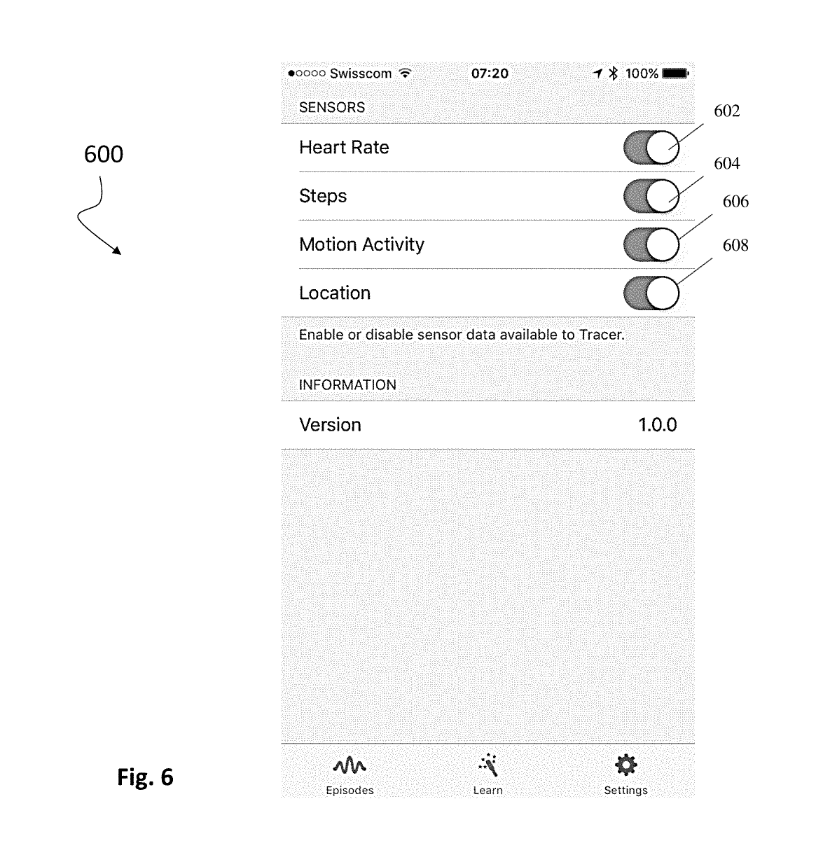

As noted herein, sensors can be used to collect information and activities can be categorized based at least in part thereon. For example, and with reference to the display screen 600 shown in FIG. 6, graphical screen controls can be provided for users to enable one or more sensors. Selections can be made to activate and/or identify respective sensors that are active. Heart rate 602 identifies information from a suitable device that can detect and/or store regular, intraday heart rate information, such as via APPLE WATCH. Steps 604 identifies step count information from any suitable device that can detect and/or store regular, intraday step count information, such as via APPLE WATCH and IPHONE. Motion activity 606 identifies motion information that can be received from a suitable device, such as IPHONE. In one or more implementations, a determination can be made to detect, for example, that a user is stationary, walking, bicycling, traveling in a vehicle, or engaged in other activity. Location 608 identifies location information. that can be received from a suitable device, such as via an IPHONE. In one or more implementations, low-accuracy, background modes are used to reduce a drain on the battery and do not rely heavily on GPS. Such modes can include location hints from change of cell towers as well as visits to places as determined by a respective device, such as an IPHONE.

In one or more implementations of the present application, data are broken down into episodes of 10 minutes. This results in six episodes per hour, and up to 144 episodes per day. An episode may only be generated if there are heartrate data and/or step data. Recent episode data (e.g., the most recent 1,000 episodes) can be supported. Various episode data can contain the following:

Date. The start of the respective episode.

Heart rate. The average heart rate during the respective episode.

Steps. The step count for the episode. In case multiple devices report step data, the app selects the device with the highest contribution for the respective episode.

Motion Activity. The motion activity for the respective episode.

Location. The course location for the respective episode.

Distance. The course distance change from the previous episode.

Inferred activity. The activity that has been statistically inferred by the trained classification model.

Certainty. A certainty of the above inference, 0 to 1.

Confirmed activity. The confirmed activity of the episode. This activity is set manually by the user and forms the ground truth for training the classification model.

In one or more implementations, users are prompted to confirm a number (e.g., 50) data associated with episodes, which are usable to train an initial classification model. Episodes are preferably confirmed for a classification model to infer such episodes in the future. For example, when a user takes a few steps inside an office building, the user confirms the episode as “none,” and not as “walking,” on the grounds that the user does not want such limited exercise to be categorized as a walking workout. Accordingly, the value “none” represents inactivity.

Further, the present application supports mapping terms that would otherwise have no associated meaning. For example, a user plays badminton and not tennis. In such case, the user can confirm episodes in which (s)he plays badminton by selecting a value representing tennis. In this way, exercise that occurs during badminton is recognized and accounted for.

In one or more implementations, users are prompted to confirm episodes in which activity occurs for an uninterrupted amount of time, such as 10 minutes. For example, a user goes for a walk from 14:15 to 14:45, confirms the episodes at 14:20 and 14:30 as walking, but does not confirm the episodes at 14:10 and 14:40. Such episodes are considered “dirty,” and unsuitable as the ground truth for training the classification model. In such cases, users are prompted to classify the exercise as “unconfirmed.” In one or more implementations, the classification model can infer activity in response to receiving several user classifications associated with a particular activity, some of which may be inconsistent, and thereafter inferring a classification based on a majority of the user-based classifications that were received.

In the event that a user unintentionally confirms an episode, one or options can be provided for the user to clear the confirmation and, thereafter, reconfirm the episode and/or confirm a future episode associated with the same activity.

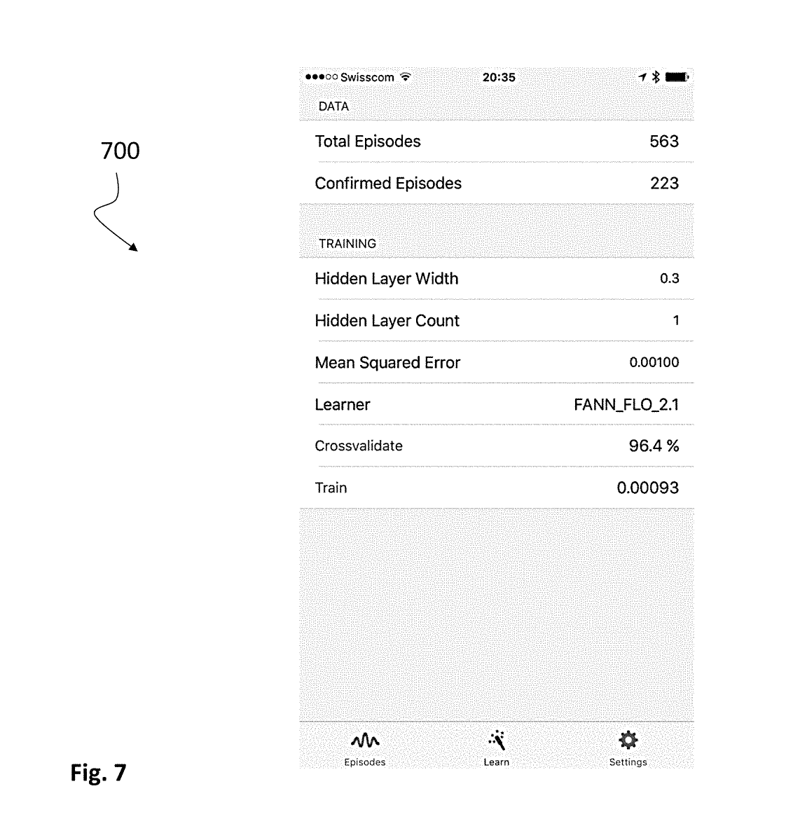

After a predetermined number of episodes (e.g., 50 episodes) have been confirmed by a user, and at least 25 of which are classified as “none,” the user can be prompted to further train the classification model. Options provided in an example training interface are shown in display screen 700 in FIG. 7. The trained model can be implemented in an artificial neuronal network (“ANN”). The model is based on the up to 10,000 most recent episodes in the app, and immediately applied those episodes, including confirmed and unconfirmed ones. New episodes can be classified automatically based on the model.

Periodically, users can be prompted to re-train the model, such as when the user initially interacts with the system and/or confirms one or more episodes. In such case, a user can be prompted to review previously classified episodes and to make revisions to such classifications as appropriate. For example, users can be prompted to decide whether an inference is wrong based on the sensor data points of the episode. Users can be further prompted to determine whether more confirmed episodes for one or the other activity would increase accuracy.

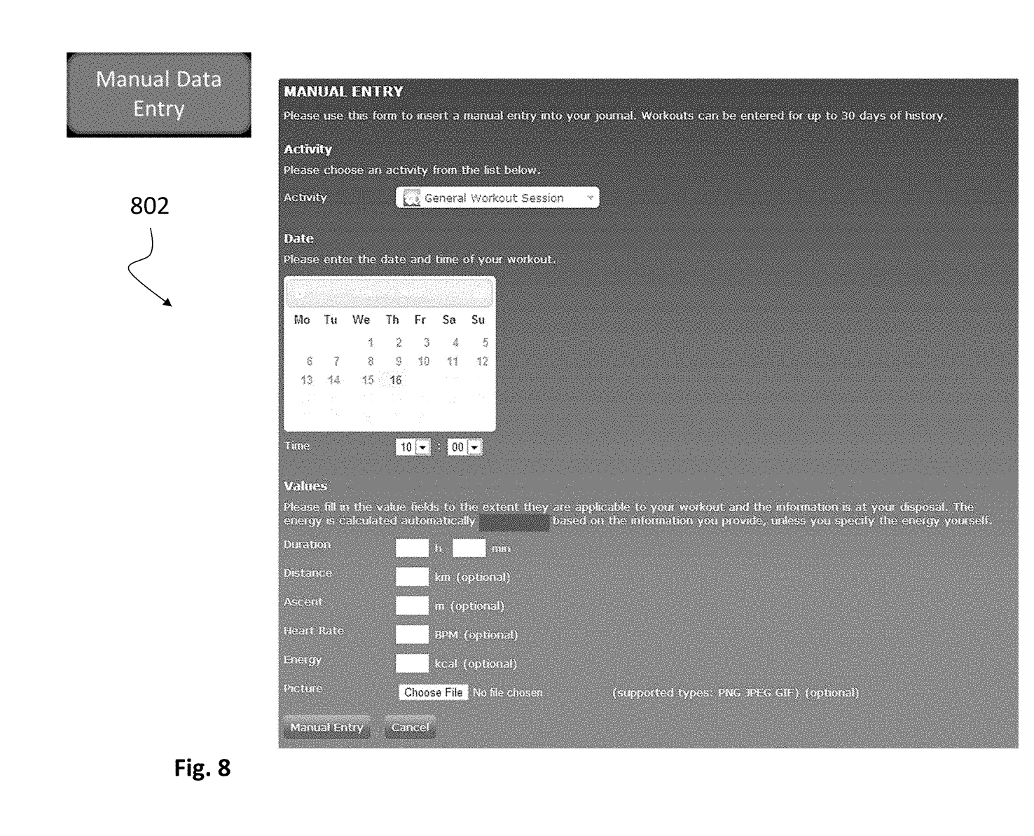

An example interactive data entry display screen 802 is shown in FIG. 8, which includes graphical screen controls in a user interface provided, for example, on device 104 and that enables the user to enter activity information, calendar information, and respective values associated with duration, distance, heart rate, energy and picture. These, for example, are usable for manual data entry in connection with the teachings herein.

Once a user has confirmed a predetermined number of episodes, such as at least 200 episodes, at least 100 of which are classified as none, the user can engage in a process to cross-validate the episodes. For example, a 10-fold cross-validation, using 90% of the confirmed episodes, can be used as a training set and the remaining 10% as test set. The process can be repeated 10 times, using a different 10% of the data as test set each time. After this cross-validation process is complete, the accuracy of the model is shown and formatted as a percentage. Occasionally, a user may repeat cross-validation steps a few times to properly gauge the accuracy, as there may be a certain degree of randomness, especially for one or more activities that have very few confirmed episodes.

In case a user sees a low accuracy (e.g., <90%), the user may want to configure hyper parameters of the ANN: One hyper parameter is a hidden layer width. The hidden layer width is relative width of the hidden layers compared to the input layer, 0 to 1. Wider hidden layers allow the algorithm to learn more complex patterns, but may lead to overfitting, thus lowering the accuracy.

Another example hyper parameter is hidden layer count. This is the number of hidden layers, 0 to 4. More hidden layers allow the algorithm to learn more complex patterns, but may also lead to overfitting, thus lowering the accuracy.

Another example hyper parameter is mean squared error. This is the stop threshold, 1 to 0.00001. Network training stops once the mean squared error over the training set has fallen below that threshold. A lower threshold allows the algorithm to train to a higher degree of precision, but, again, may lead to overfitting, thus lowering the accuracy.

After changing one or more hyper parameters, the user can re-run cross-validation one or more times to see if there is an improvement or a degradation in accuracy. When changing a mean squared error, the user may try orders of magnitude first, i.e. 1, 0.1, 0.01, . . . , 0.00001. In one more implementations, the default values of the hyper parameters can be 0.3 hidden layer width, 1 hidden layer, and 0.001 mean squared error threshold.

### “Pay as You Live” and Insurance Implementations

In one or more implementations, the health score can be integrated in insurance products, such as health insurance, vehicle insurance and life and/or health insurance. Using the systems and methods disclosed herein, including in connection with the generation and reception of information substantially in real-time via on-line and/or mobile platforms, insurers can identify risk exposure and provide for price controls for users accordingly. For example, one or more sensors, such as biosensors, can be used to collect and transmit health information about a user who has purchased health insurance. The one or more sensors can be configured in a health band, such as shown and described herein, to collect health and activity information about the user. Further, the tracker application can track fitness activities, and generate and transmit activity information to one or more computing devices. Introducing such technology into the insurance and/or reinsurance industry can facilitate an entirely new user experience and provide for significant cost savings for both the insurer and the insured. For example, information such as relating to respiratory activity, steps and other physical activity, heart rate, sleep, nutrition, stress, blood sugar and food intake can be generated and transmitted to one or more computing devices and used for generating notifications for respective parties, such as insured users. Such notifications can alert users to various conditions, and lead to changes in behavior.

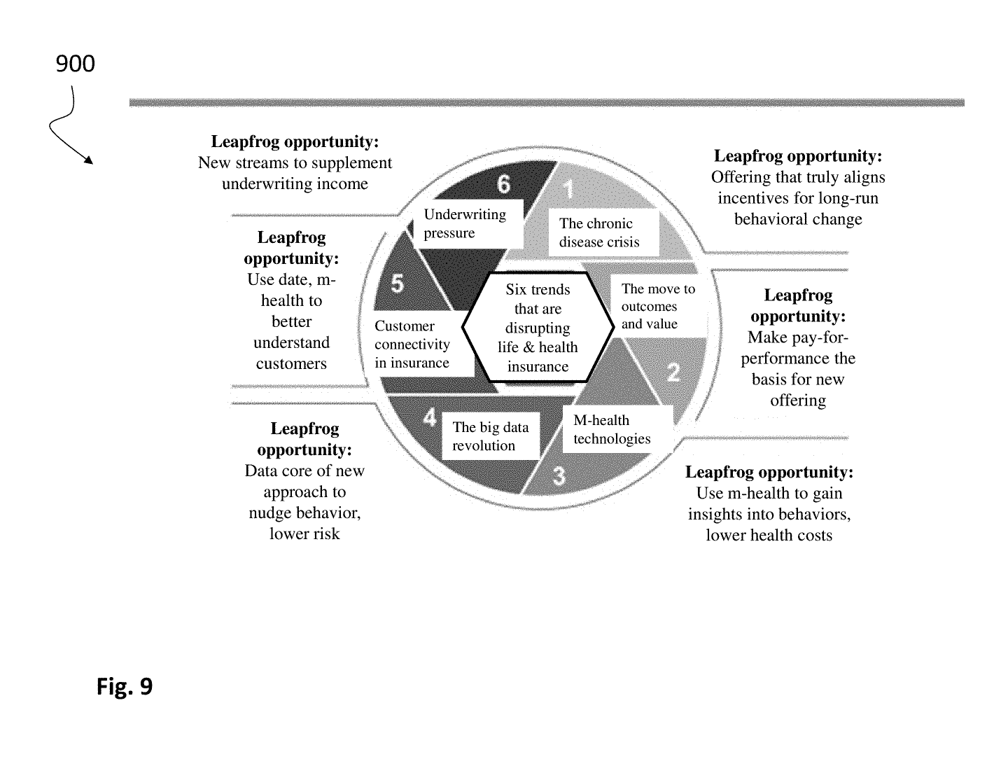

FIG. 9 is an example graph 900 that identifies six opportunities that can be provided in accordance with one or more implementations of the present application. For example, opportunity can be provided in accordance with the present application by providing offerings that align incentives for long-term behavioral change, such as in connection with chronic disease and pain management. One or more modules implemented by the present application support new insurance offerings based on a pay-for-performance model. This opportunity provides for development of new insurance products that are contingent upon outcomes and corresponding value to a user. Furthermore, mobile health (“mHealth” or “m-health”) technologies such as shown and described in herein can be employed to provide usable information that can gain insights into behaviors of insured people, improve outcomes and lower costs.

Thus, the teachings herein effectuate a complex data core as a function of integrated monitoring technologies, and that encourage insured people to improve behavior and lower health risks. Further, the health of insured people can be better understood as more information attributed to respective lifestyles is obtained and processed by the present application. The result can be, for example improved income streams to supplement underwriting income, reductions of pressure to underwrite insurance policies, and significant cost savings for insured users.

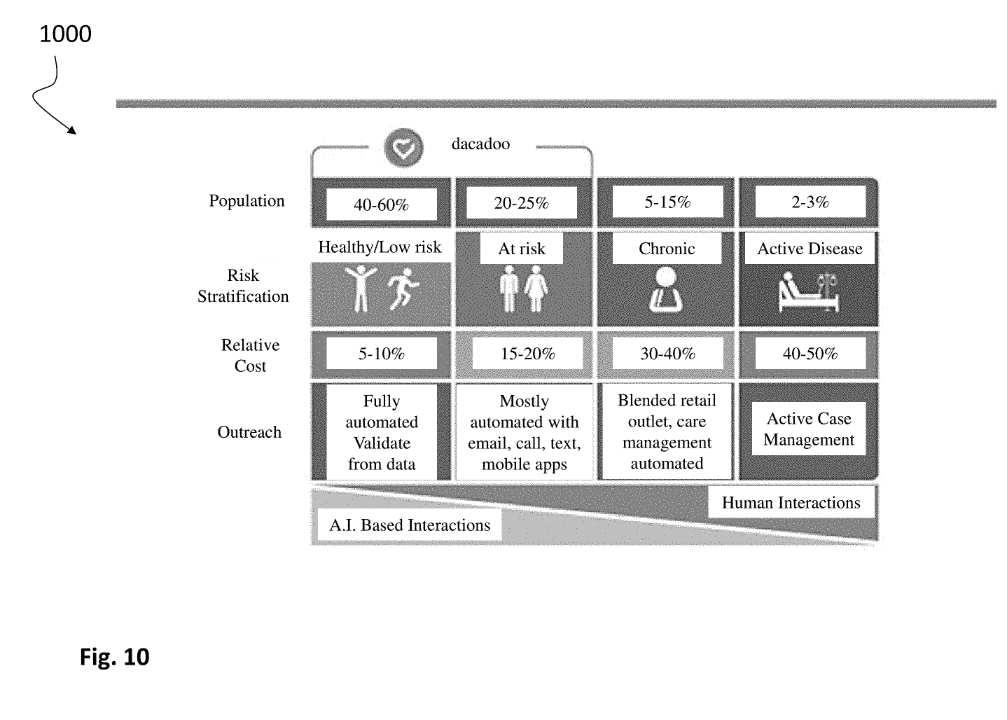

FIG. 10 illustrates an example chart 1000 that identifies, generally, populations that can be serviced well by proprietors of the present application. For example, a relatively large percentage of the population, 40 to 60% as shown in FIG. 10, are well served by artificial intelligence-based interactions, and having a fully automated outreach. Risk stratification for such percentage of the population is relatively low, as members are relatively healthy. Accordingly, this percentage of the population bears a relatively low cost of health and/or life and/or health insurance. Also well served by the teachings herein is a significant sector of the population, shown generally as 20 to 25%, who are at some health risk, and bar a somewhat larger percentage of insurance-related costs, such as 15 to 20% of the total costs. The outreach to this percentage of the population is well-automated, including as a function of email, cellular phone communications, chat bots, SMS text and online mobile applications. Also represented in the chart 1000 is a smaller percentage of the population, such as between 5 to 15%, are among higher risk, and may endure chronic health and pain issues, and who bear larger percentage of total insurance-related costs, such as 30 to 40% of the total. This percentage of users may be reachable via some forms of technology, with a significant portion of information representing this percentage of users being provided from care management centers and/or medical professionals. Moreover, a relatively small percentage of the population, illustrated in chart 1000 as between 2 to 3%, are not necessarily well-served by the technological features shown and described herein. This small percentage of the population may bear 40 to 50% of total insurance-related costs, and suffer from chronic disease and require active care and case management.

Thus, the present application provides opportunity and implementations for new insurance-related products that are configured to factor M-health information including health scoring, and move toward real-time lifestyle based underwriting and new products. Insured individuals can benefit from the teachings herein as behavior and activity is monitored, and information is generated and transmitted to one or more computing devices associated with insurers, substantially without human interaction, which increases speed and efficiency backspace. Insurers benefit, as well, as the health and state of insured parties (and insured things) remains healthy, thereby increasing the length of time that premiums will be paid.

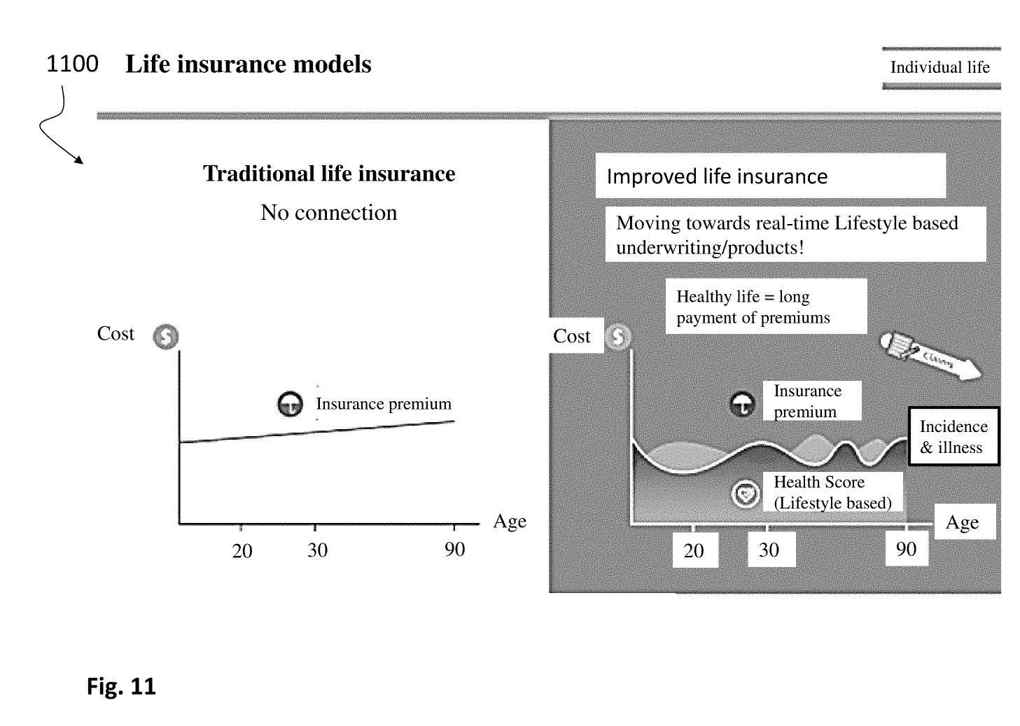

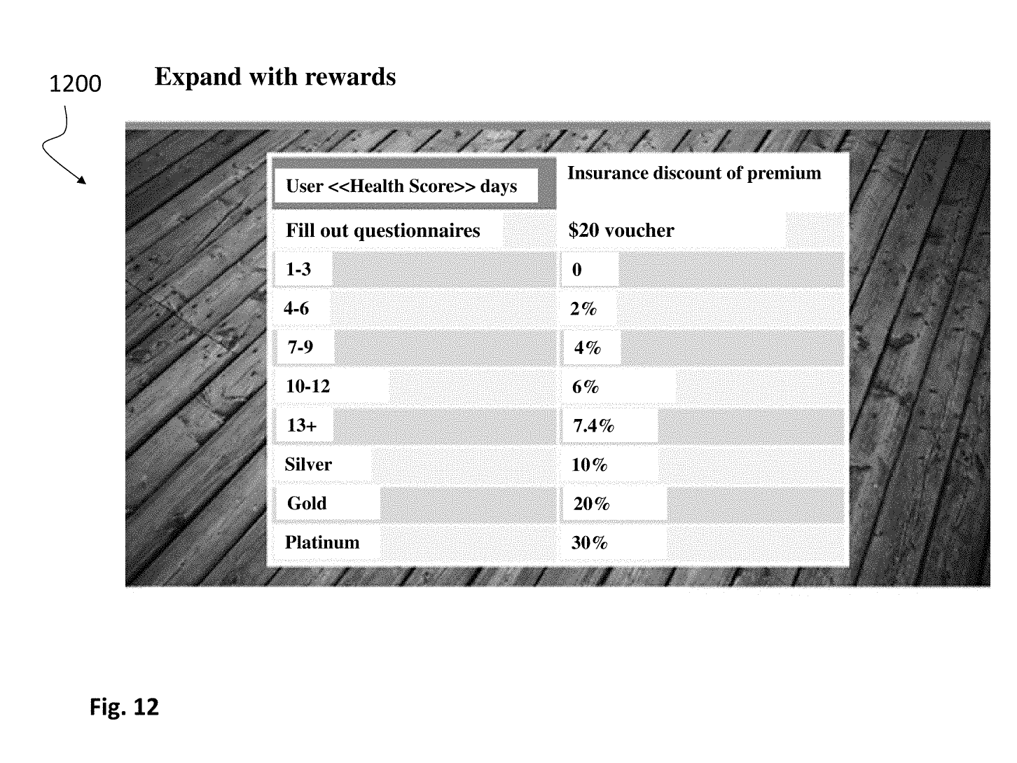

FIG. 11 illustrates example graphs 1100 showing traditional life and/or health insurance and the improved life and/or health insurance offerings of the present application, in which insurance costs are represented (Y-axis) and insurance premium payments over time is represented (X-axis). In the traditional insurance model, the result is a relatively straight line, static result. In the improved life and/or health insurance of the present application, however, a dynamic nature of real-time lifestyle information is represented. The dynamic relationship represented in the improved life and/or health insurance graph represents incidents and illness, as a function of information being regularly and automatically received from one or more on-line and mobile platforms. Furthermore, health scores are calculated substantially in real-time, and premium rates are determined in response, such as for new insurance products and/or to adjust rates in existing ones.

Accordingly, and in one or more implementations, behavior and physical conditions of people can be automatically monitored, and information can be dynamically and regularly provided, such as to calculate real-time health scores. This enables insurance companies to develop lifestyle-based insurance products, such as in connection with health insurance and life and/or health insurance, that are far more accurate, provide better value for users, and increase profitability for companies. For example, the present application implements block chain and electronic information transfer technology in substantially automated insurance offerings. The present application enables companies to assess risk that would otherwise be impossible to detect, and improved underwriting and pricing resources can be developed to ensure a comprehensive underwriting and pricing strategy that is sustainable and that warrants long-term profitability and growth. Using the technology shown and descried herein, users have access to real-time information that assist them to make that reduce risk and positively affect outcome-based fees and/or benefits.

In one or more implementations, information received and processed in accordance with the teachings herein can be applied to determine discounts, rebates or other financial benefits associated with insurance premiums. FIG. 12 illustrates an example chart 1200 that identifies percentage discounts offered to users as a function of information actively provided by a user, such as in response to questionnaires, or one or more interactive mechanisms (e.g., data entry forms). Upon reaching a certain level, such as by responding to 13 or more individual questionnaires over time, a user's status can increase (e.g., to silver, gold, platinum) and the benefits offered by the present application, such as percentage discounts on premiums, can correspondingly increase.

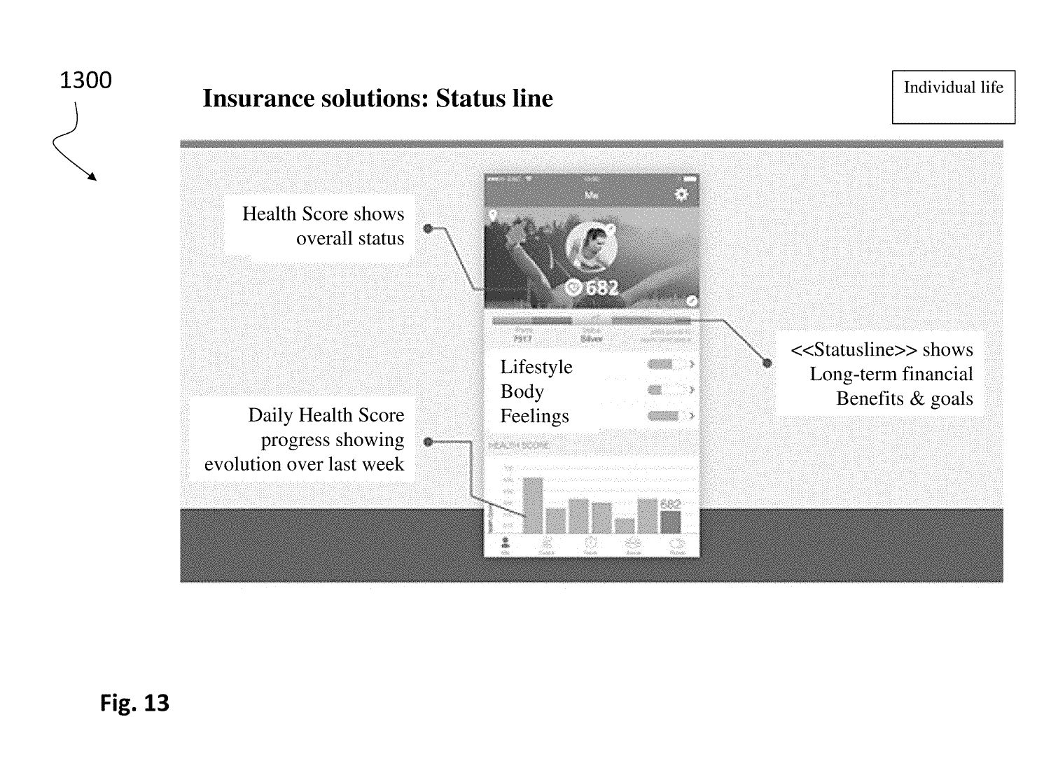

In addition to information received in response to questionnaires, other information can be attributed for defining a respective user's status and impacting benefits, such as percentage discounts on premiums. FIG. 13 illustrates an example display screen 1300 that is provided on a mobile computing device and that identifies a respective user's health score at the present time, as well as a bar graph identifying progress of the user's daily health score over a previous week. Additionally, graphical screen controls are illustrated that provide feedback associated with lifestyle, body and feelings, as shown in display screen 1300. Further, display screen 1300 includes a status line that shows long-term financial benefits and goals attributed to the health score and its impact on the user's insurance premiums.

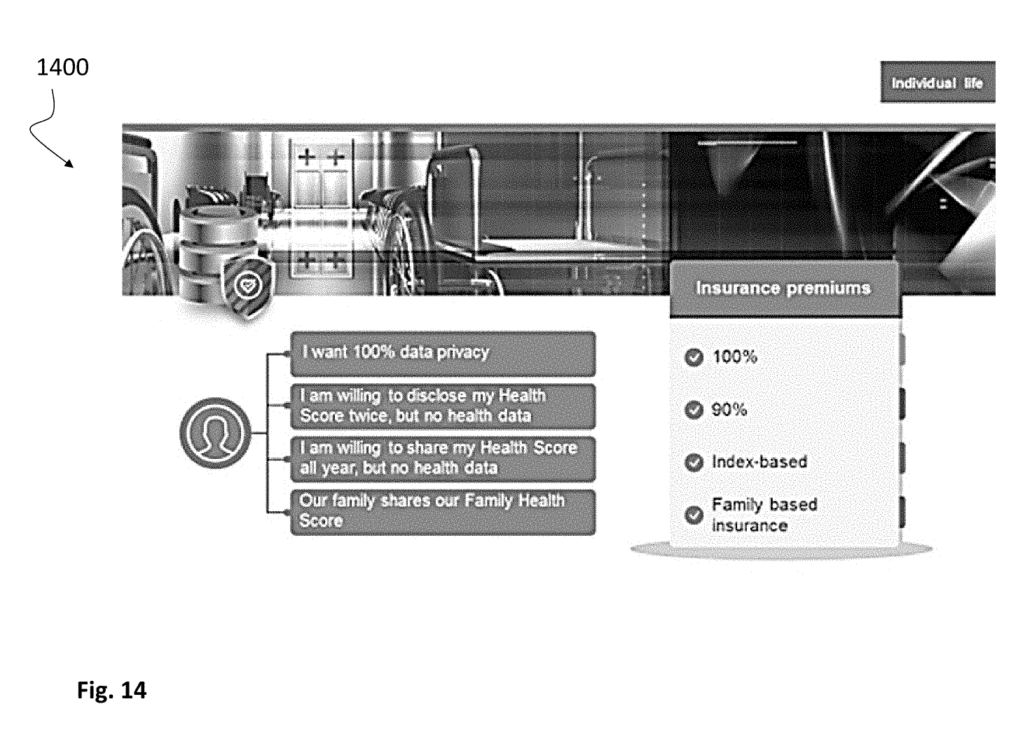

It is recognized by the inventor that some users of the present application may be willing to forego some financial benefits, such as rewards or discounts on insurance premiums, in exchange for greater control over the users' personal health information and data privacy. FIG. 14 illustrates an example data entry display form 1400 that includes options for a user to select in connection with maintaining control over information and privacy. As shown in display screen 1400, options are available for user to maintain data privacy, an option to disclose health score a limited number of times (e.g., twice) but without disclosing health data, such as that is used in the calculation of the user's health score. Another selectable option enables a user to submit that (s)he is willing to share the health score for a given period of time, such as an entire year, but is not willing to share any of the health data that is used in the calculation of the health score. Yet another selectable option illustrated in example display screen 1400 enables a user to identify that his or her family shares a family health score, which can be a calculated aggregate value representing the family's health at large, without identifying or disclosing any individual member of the family's health score. Also illustrated in display screen 1400 is a chart identifying percentages of insurance premiums, including 90% twice per annum, index-based and family-based insurance, that are identified as being directly impacted by the amount of information a user is willing to share or otherwise disclose publicly or in limited contexts. Accordingly, the present application provides substantially persistent and real-time information that improves automated underwriting/risk modeling and rewards customers accordingly.

In one or more implementations, the present application processes health score and/or related information associated with a plurality of people in the aggregate. For example, information processor 102 and/or user computing device 104 processes user information associated with a plurality of users in the aggregate and can define bands or tiers of users accordingly. An example of such bands can be defined as shown in display screen 100 (FIG. 10), and in display screen 1100 (FIG. 11).

For example, a band of individuals deemed very healthy and extremely low-risk as a function of at least their respective health scores satisfy the “platinum” user band and are offered a maximum discount on insurance premiums, such as 30%. Other respective user bands or tiers, as defined or represented in connection with users' health scores, can be offered other corresponding benefits, such as discounts on insurance premiums. In various implementations, other types of information such as representing users' responsiveness, lifestyle, activity or the like can be factored into the aggregate user groups for defining user bands and determining respective individuals that belong thereto. Processing respective user information, such as relating to health scores, in the aggregate improves upon known conventional computing and technological processes associated with underwriting/risk modeling and providing customer benefits that directly result therefrom.

Various implementations of the present application that include a pay-as-you-live insurance model, such as shown and described herein, and that process information in the aggregate for multitudes of people can pass cost savings associated with improved technological functionality to customers, while affording improved profitability for insurers. For example, 30 bands or tiers of users can be defined as a function of various medical criteria, including the users' health scores and/or health score ranges as shown and described herein. Users can be informed that pluralities of tiers or bands exist, and that varying degrees of benefits (e.g., discounts on premiums, increases in coverage, or other benefits) are associated with the respective bands. This incentivizes users to improve their health scores, for example, by eating right, exercising regularly and following medical advice, thereby lowering their overall risk levels for insurers.

Once users have been categorized within a respective band or tier, mobility into another band such as one that provides a greater or lesser degree benefit can be achieved. It is recognized, however, that users may engage in healthy activities in an effort to increase their respective health scores with the hope of moving into more profitable bands, but that such improved behaviors often taper off over time. It would be inefficient to process information associated with thousands or millions of users over and over, re-categorizing the users back and forth into various bands as behavior habits that affect health scores change. Accordingly, the present application supports a mechanism to detect when user behavior improves (e.g., engages in activity to improve his or her health score) and thereafter implements a module to delay altering the user's categorized user band for a period of time or in accordance with other predetermined criteria. In one or more implementations, a mechanism is supported that detects when a user's health score improves and begins a process for tracking the user over time for continued and consistent improvement. Further, the present application can be configured to recognize patterns of behavior that may misrepresent a user's behavior and health trend over time. For example, a user may consistently exercise, eat right and follow medical advice at the beginning of each month, but by the end of each month the same user is not exercising, eating foods that are unhealthy and not taking prescribed medications. It would be inefficient to move this user in and out of a higher tier or band each month as the user cycles through this behavior.

The present application improves upon computing and processing technology by including and exercising a form of a dampening mechanism to preclude changes in a user's respective band or tier. For example, after a user trends positively for a period of time e.g. a month, three months, six months or more, then the user can be migrated out of his or her current user band and into a different one. An example of detecting activity is provided in display screen 1300 (FIG. 13), which represents a spike in the user's health score at the beginning of the week, followed by a reduction in the user's health score progress over time. Using the dampening mechanism of the present application, the user is not categorized in a different tier or band as the inconsistency in the user's health score is revealed.

In one or more implementations, the present application supports a messaging interface that can be configured for respective tiers and/or bands of users, as well as for users who are engaged in various activities or lack thereof. For example, a database accessible by information processor 102 and/or user computing device 104 can store messages or elements thereof associated with respective bands of users. Criteria associated with user behavior, user health scores, user bands and/or tiers can be used to identify respective messages and/or elements thereof in a database to be retrieved, assembled and/or transmitted to users. For example, a message can represent that the user has been active in respect in various ways and has been improving his or her health score. The messages can be provided via the feedback loop and formatted in many ways, such as text, chat bot, voice or other suitable mechanism, such as shown and described herein.

Thus, as shown and described herein, outcome-based insurance is supported using the technological features and advantages shown and described herein. Use of the health score, for example, can be particularly integral for identifying risk and calculating adjusted insurance premiums initially and/or over time for users. The present application offers users and insurance companies competitive advantages as a function of our underwriting research and development services, as well engaging in feedback to help users make better decisions that reduce risk. In one or more implementations, computer transmissions are limited based on a reclassification of each of a plurality of users at least as a function of masked numerical scores received via a data communication network, over time.

### Health Score Range Implementations

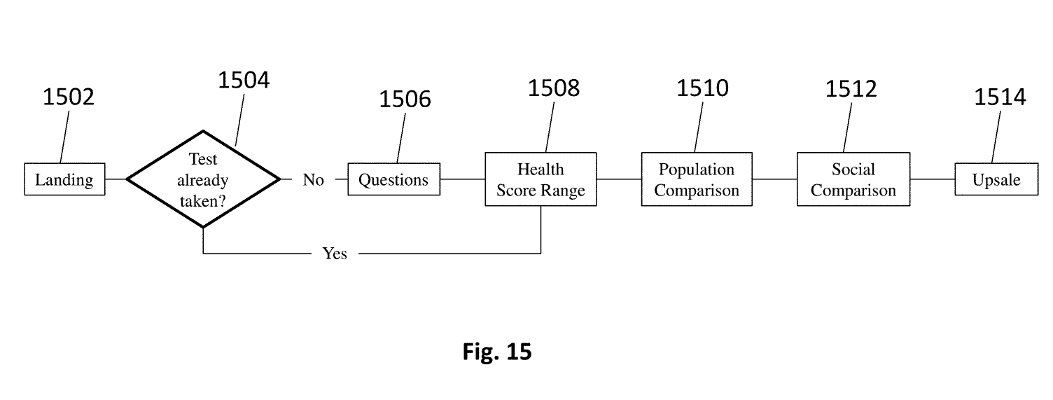

In one or more implementations, the present application supports generation and display of a range associated with a health score. The health score range can be provided in a software application, for example, that integrates with one or more publisher web sites, such as a social network. One goal is to generate and/or encourage awareness of users' health scores in public forums, as well as to encourage increased social and user interaction, including via a no-cost and/or for pay subscription-based model. FIGS. 15-23 identify one or more implementations for engaging a user to provide various information, for calculating and using a health score range publicly.

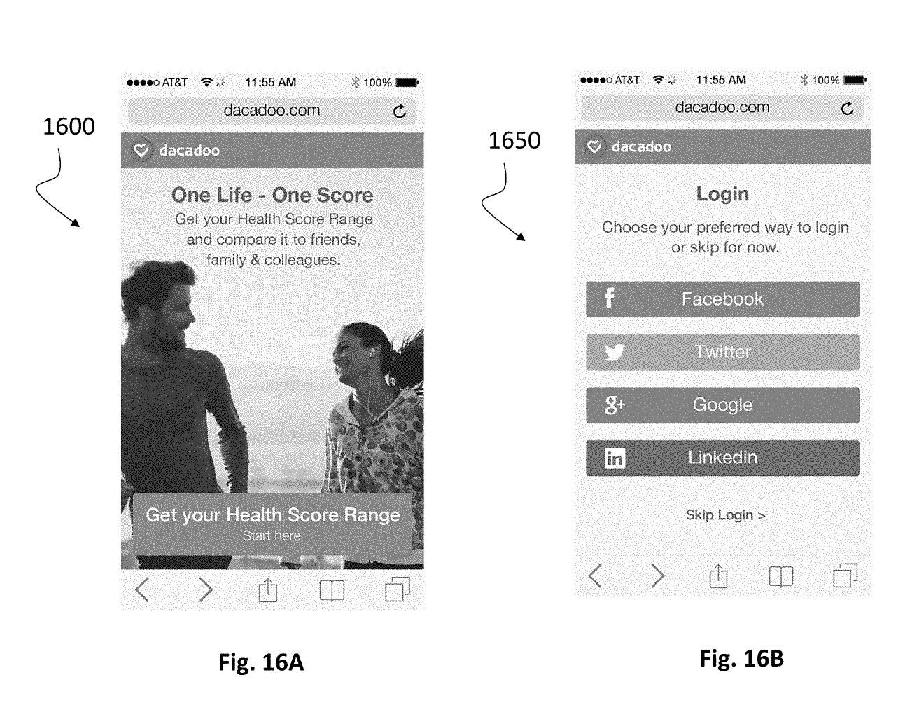

FIG. 15 illustrates an example flow of information and steps associated with generating and using a health score range in accordance with one or more implementations of the present application. At step 1502, a “landing” page is provided, which may be via an online Internet-based site, a mobile app, or other suitable interactive platform. An example landing page display screen 1600 is illustrated in FIG. 16A, which includes one or more graphical screen controls for a user to begin the process of generating a health score range. The landing page can introduce options to login to a user's account via one or more social networks (e.g., Facebook, Twitter, Google or LinkedIn). Alternatively, the user can skip the login process and proceed to enter information to generate the health score range. An example login data entry display screen 1650 is shown in FIG. 16B. The landing page can be optimized with “Open Graph” and “Twitter Card” tags, for example, to ensure a suitable rendering when shared on social networks. Examples can include ogp.me, dev.twitter.com/cards/overview or the like. Furthermore, the landing page can be opened via a simple uniform resource locator (“URL”), or via a more complex URL, such as formatted to contain a respective user or other ID. The respective ID can be referenced in one or more database, and personalized information can be accessed and entered vis-à-vis the Open Graph and/or Twitter Card tags. This enables personal information to be rendered when a user shares his/her ID on a respective social network. Thereafter, when other users select (e.g., click on) a shared link, the other users can be “sent” to the Landing page where they can be prompted to enter information for respective health score ranges. In one or more implementations, only minimal amount of information is requested of a user, such as a respective ID, identification of one or more “friends” that are active using the health score Range application, a username, and/or a profile picture.

At step 1504 (FIG. 15), a determination is made whether a user has already submitted information suitable for calculating a health score range (taken a “test”). If not, then the process continues to step 1506 and one or more questions are provided to the user.

In one or more implementations, a test is assigned a random variable, e.g., the value “1” with values “A” to “C.” the variable is usable to determine a title and description used in the Open Graph and Twitter Card tags. Table 1 shows variables in an example implementation using a test ID in accordance with the present application.

TABLE 1

| Test | Test | | | |
|---|---|---|---|---|
| ID in | Random | | | |
| URL | Variable 1 | Tag Title | Tag Description | Tag Image |
| No | N/A | dacadoo Health | Take the dacadoo Health Score Range test and see how healthy you are | Generic Health Score |
| | | Score Range | compared to friends, family and colleagues. One life, one score. | Range Image |
| Yes | A | My Health Score | <name> has taken the Health Score Range test and got into the Top | Personalized Health |
| | | Range is <range> | <persentile> %. Can you beat that? | Score Range Image |
| Yes | B | <name> is in the | <name> has taken the Health Score Range test. What is your score? | Personalized Health |
| | | Top <percentile> %. | | Score Range Image |
| Yes | C | One Life - One Score | Do it like <name>, and find out how healthy you are with the dacadoo | Personalized Health |
| | | | Health Score. | Score Range Image |

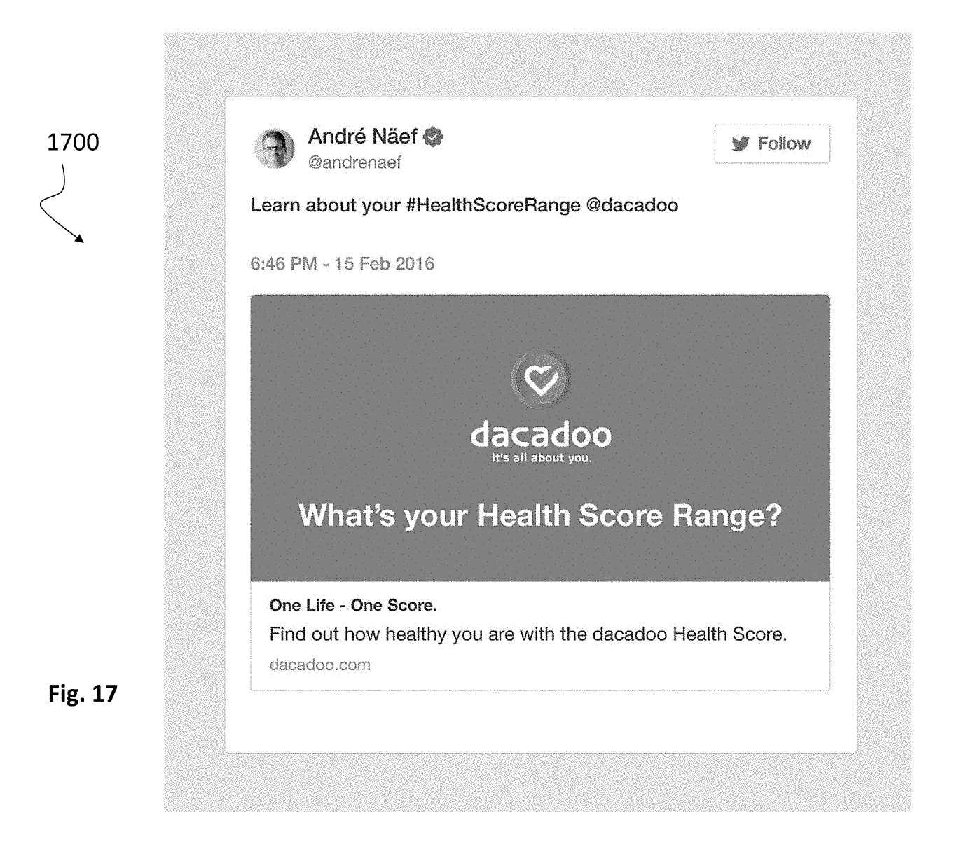

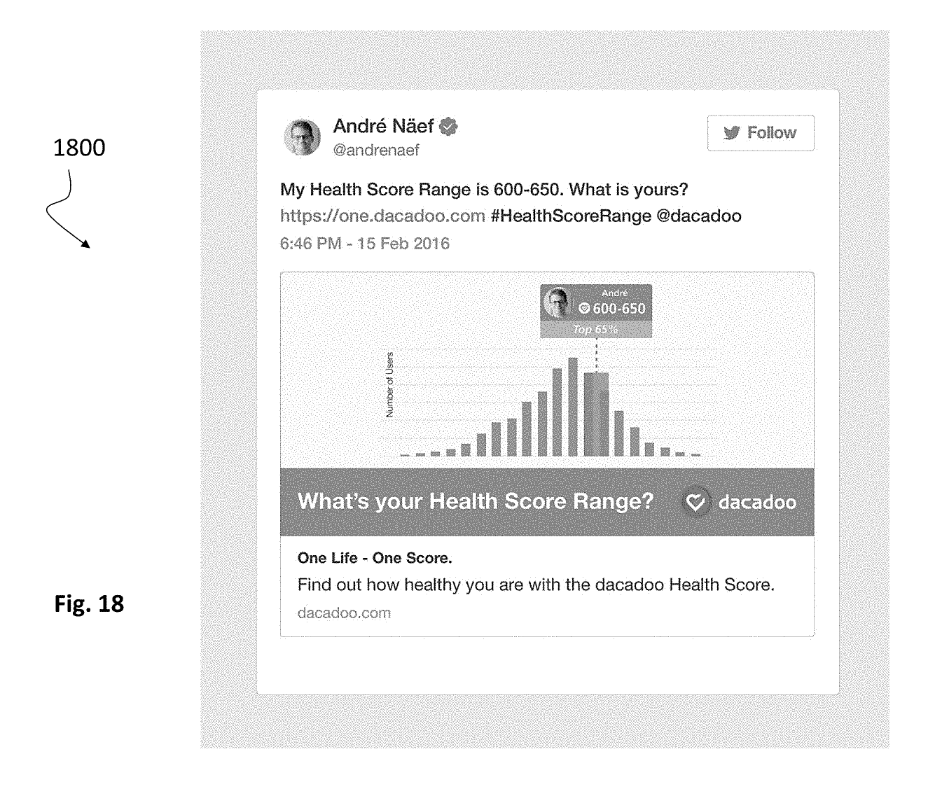

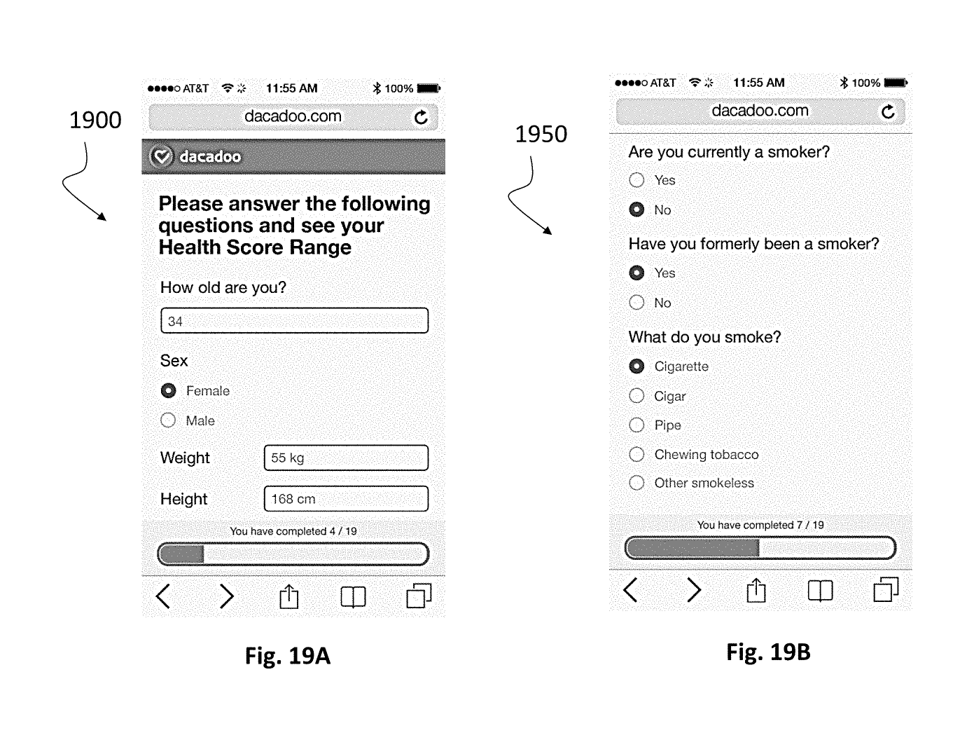

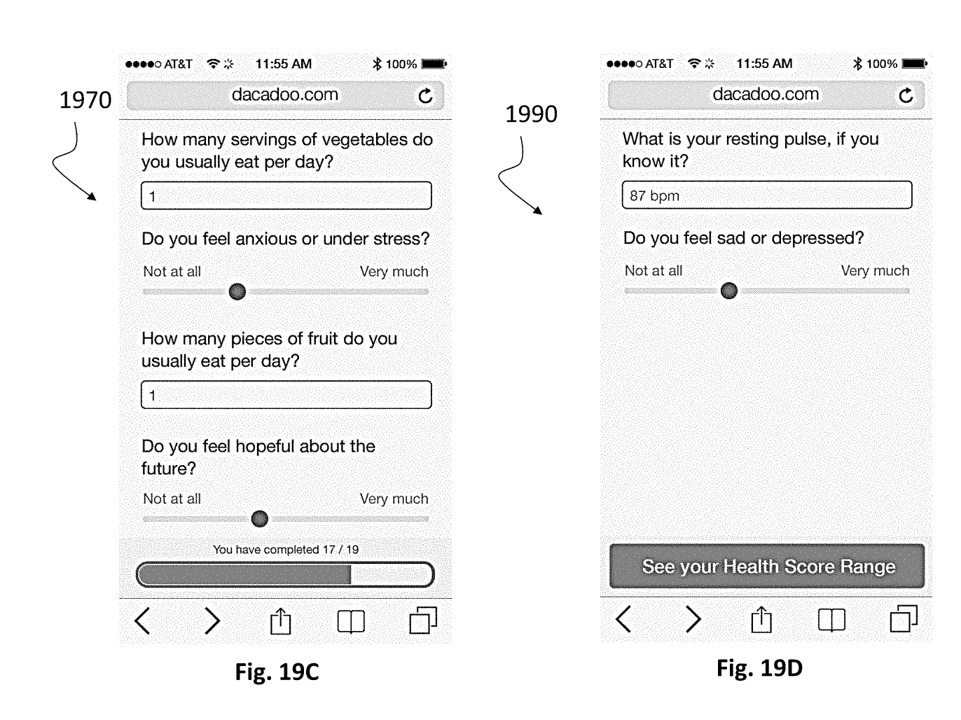

Upon completion of a suitable login procedure, a series of prompts can be generated via the mobile computing device, a user can be prompted to begin the process of calculating his or her health score range vis-à-vis a social network or other publisher site, such as shown in display screens 1700 and 1800, FIGS. 17 and 18, respectively, as the process continues to step 1506 (FIG. 15). Thereafter, a plurality of data entry display screens can be provided that include questions for the user to respond to. Example display screens 1900 (FIG. 19A), 1950 (FIG. 19B), 1970 (FIG. 19C) and 1990 (FIG. 19D), illustrate an example data entry interface in which a user is requested to answer information, such as relating to age, gender, weight, and height. Other information requested of the user can include habit-based information, such as smoking and drinking, types of food consumed, and subjective feelings information, such as whether a user feels anxious or hopeful.

Table 2 shows an example implementation including questions, conditions and data input types, in accordance with providing a “test” to a user in connection with generating a health score range.

TABLE 2

| ID | Question | Condition | Unit | Input |
|---|---|---|---|---|
| 1 | How old are you? | | years | Number |
| 2 | Sex | | male, female | Buttons |
| 3 | Height | | cm or in | Number |
| 4 | Weight | | kg or lb. | Number |
| 5 | Are you currently a smoker? | | Yes, No | Buttons |
| 6 | Have you formerly been a smoker? | (5) = no | Yes, No | Buttons |
| 7 | What do you smoke? | (5) = yes | Cigarette, Cigar, Pipe, Chewing tobacco or Other | Buttons |
| | | | smokeless | |
| 8 | How many of these products do you consume per day? | (5) = yes | 0 to 30 | Number |
| 9 | What did you smoke? | (6) = yes | Cigarette, Cigar, Pipe, Chewing tobacco or Other | Buttons |
| | | | smokeless | |
| 10 | How many of these products did you consume per day? | (6) = yes | 0 to 30 | Number |
| 11 | How many years ago did you start smoking? | (5) = yes or | >0 | Number |
| | | (6) = yes | | |
| 12 | How many years ago did you stop smoking? | (6) = yes | >=0 | Number |
| 13 | How much do you exercise per week? | | None, Up to 1.5 h, Up to 3.5 h, Up 6 h, Above 6 h | Buttons |
| 14 | How many servings of vegetables do you usually eat in a day? | | 0 to 5 | Number |
| 15 | Do you feel anxious or under stress? | | not at all to very much so | Slider |
| 16 | How many pieces of fruit do you usually eat in a day? | | 0 to 20 | Number |
| 17 | Do you feel hopeful about the future? | | not at all to very much so | Slider |
| 18 | What is your resting pulse, if you know it? | | 40 to 110 bpm | Number |
| 19 | Do you feel sad or depressed? | | not at all to very much so | Slider |

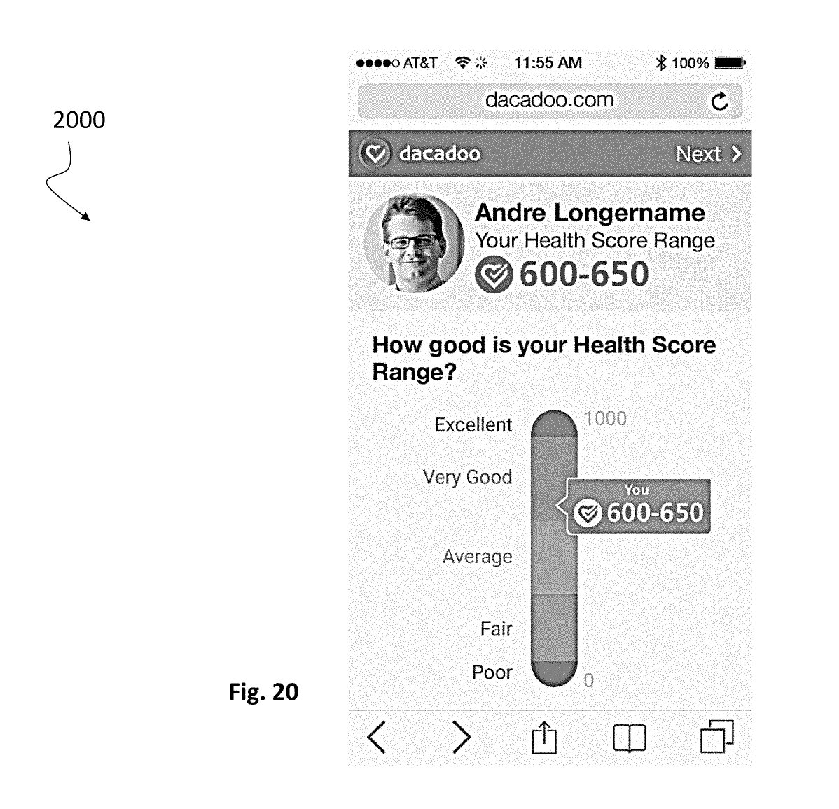

Upon receiving answers to one or more of the respective questions via the interactive data interface, such as illustrated in FIGS. 19A-19D, one or more modules operate to generate a health score range, substantially without human interaction, and to display the health score range (step 1508, FIG. 15). For example, and as illustrated in example display screen 2000 (FIG. 20), a range of 600-650 has been generated and displayed for a respective user. Moreover, a vertical chart identifying the quality of the health score range can be provided, such as shown in display screen 2000, in which the user's health score ranges between average and very good.

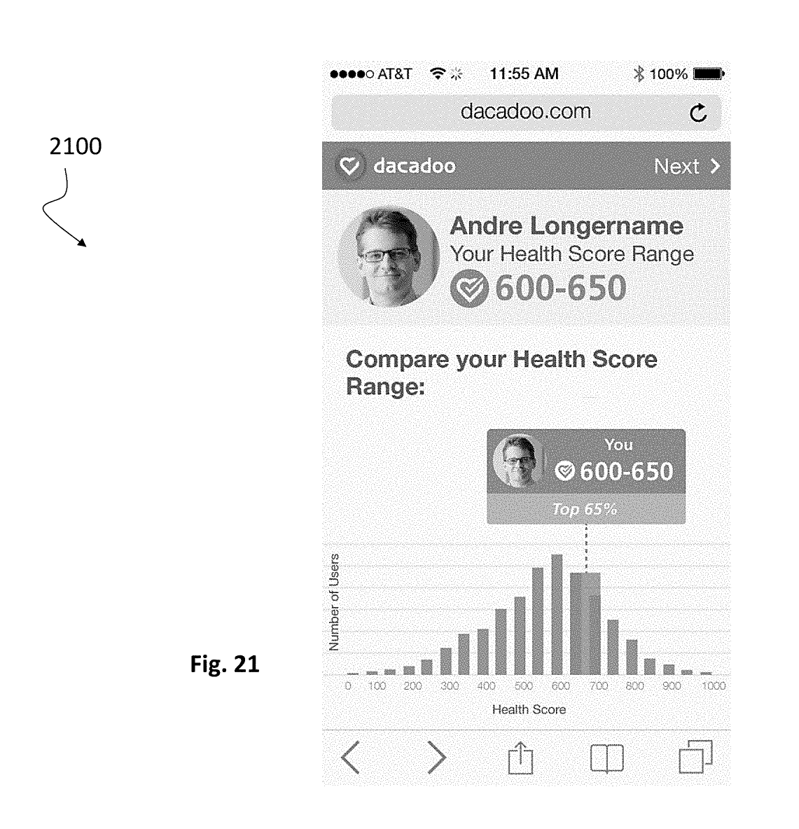

Continuing with the example flow shown in FIG. 15, a population comparison of health score ranges can be provided to the user, such as in the form of a bar graph (display screen 2100, FIG. 21). Providing a comparison of a health scare range to a user is useful feedback, such as to identify where in a percentage of the population the respective user's health score range lies. In the example shown in display screen 2100, the user's health score range is within the top 65% of the overall population.

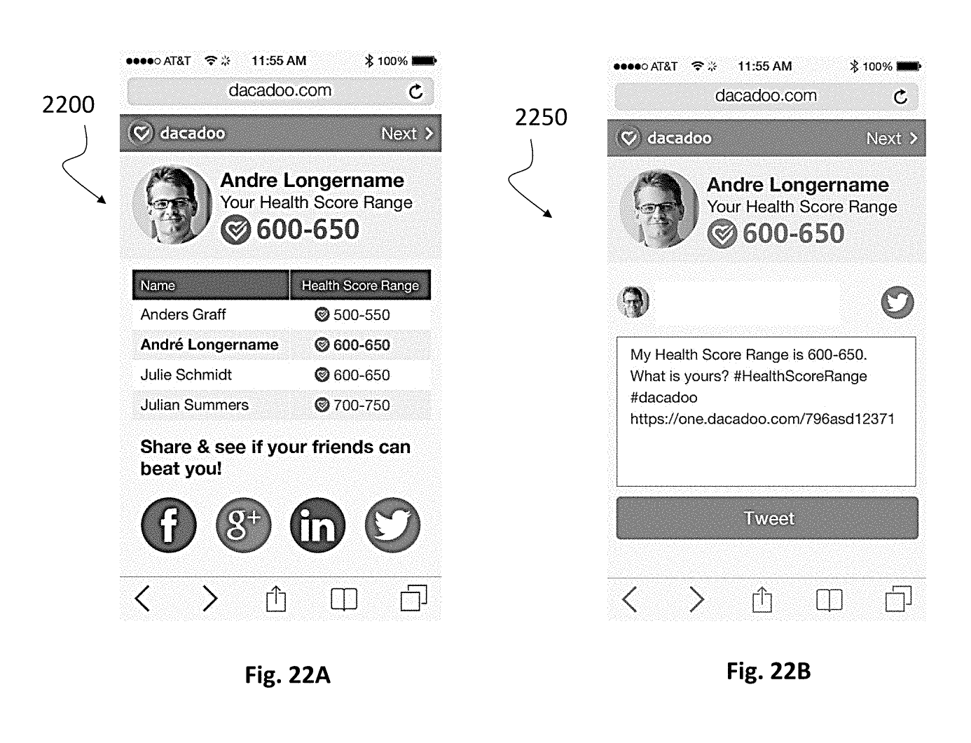

In addition to identifying a population comparison for a user, a social comparison can be provided (step 1512, FIG. 15). For example, and as illustrated in display screen 2200 (FIG. 22A), a user's health score range can be displayed in a table with other users (e.g., “friends”), which can promote competition and incentivize users to improve their health score range, and their overall health accordingly. The social aspect in step 1512 can extend in various ways, including as shown in example display screen 2250 (FIG. 22B) in which a text box can be configured to enable posting (e.g., tweeting) in a social network to other users. In one or more implementations, tweets can be automatically generated regarding a user's health score range for ease and convenience.

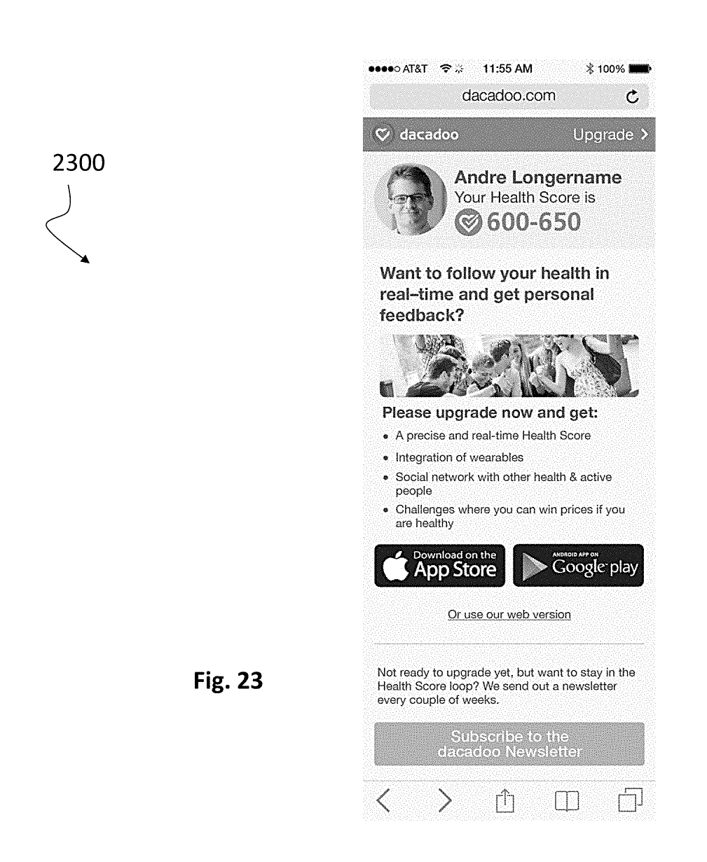

Continuing with reference to the flow shown in FIG. 15, another opportunity provided in accordance with the present application is upsale (step 1514), which can encourage and enhance interactivity, subscriptions and other social-based online activity. FIG. 23, for example, illustrates example display screen 2300 which includes a plurality of prompts for further engagement, such as for downloading and installing additional software (e.g., apps) subscribing to newsletters or other suitable content.

### Neuro Linguistic Programming Implementations

In one or more implementations, one or more modules executing neuro-linguistic programming (NLP) code, for example, in a mobile computing device provides for voice-enabled feedback. In one or more implementations of the present application, feedback such as shown and described herein relating to alerts and notifications, encouragement, and/or various kinds of valuable health-related information, including a user's health score, can be provided via voice-based technology. Such NLP modules reinforce a connection between the user's neurology, and the user's language and experiential (e.g., cultural processes), thereby encouraging behavioral change in users and to achieve specific goals, including activity-based and health-based goals.

Moreover, NLP modules executing on one or more computing devices providing voice-based interactivity are particularly useful for reinforcing patterns of behavior, including as a function of auditory-based representations, verbal communication and positive reinforcement. Such sensory-based reinforcements are attributes to behavior modification, even on a subconscious or even unconscious level.

In one or more implementations, modules executing NLP programming code include modeling that “learns” from the user's behavior and provides subjective experience for the user that contributes to modify the user's behavior, as appropriate. A user's mental state may be determined as a function of behavior that is monitored using the various components shown and described herein, and one or more verbal interventions or suggestions can be made to help guide the user to behave in particular ways. For example, a user may be told to remember to take medication, test blood pressure, get up and exercise, or even resist the urge to consume something that is considered unhealthy, based on learned patterns by the modules. Using information detected about a user's particular state in combination with a user's desired goal can result in verbal encouragement, cautions of undesirable behavior, or other feedback having a likely affect to achieve a respective outcome.

In one or more implementations, the present application integrates with existing voice-based systems, such as a function of open application programming interfaces (“APIs”), software development kits (“SDKs”), or other suitable forms of connectivity. Using one or more open platforms, the present application can integrate voice-based health scoring, lifestyle navigation, coaching and feedback. Moreover, NLP modules can implement voice-based interactivity in any of the various implementations shown and described herein in which information is provided to a user. This provides an effective approach to communication, personal development, and healthy behavior, including in the respective implementations shown and described herein.

For example, the systems and methods herein provide voice-based feedback substantially in real-time via NLP. Using the systems and methods disclosed herein, information can be received from user devices, and the information can be processed to provide various forms of feedback, such as alerts and notifications. Related information can be, thereafter, received and processed for additional feedback (e.g., a form of a feedback loop).

In one or more implementations, one or more rule engines can be provided that periodically and/or continuously process information, such as shown and described herein, and that generate notifications to users. Implementations can depend on a respective subsystem (e.g., data gathering subsystems, data communication subsystems, data processing subsystems) and one or more corresponding notification features. Moreover, one or more notification generating rule engines can be part of individual subsystems generating notifications. The notification features can include core information elements that are useful for the feedback process. Generally, notifications can include questionnaires and/or prompts for information, and can be presented by an interactive interface. The result can include an infrastructure configured for scheduling, processing, and delivering notifications over various communication channels and formats.

### Encrypted Health Information Exchange Implementations

In one or more implementations, the present application can include a decentralized and distributed system in which health information acquisition, health information storage, health information processing and secure communications are used in a dynamic bartering exchange platform. The present application can provide a computer-implemented system and method configured to acquire health-related and/or medical-related information from one or a plurality of sources, and to store securely the information after the information is validated. Moreover and in accordance with the present application, the user to whom the information represents controls over whether and how the information is sold, exchanged, shared or otherwise accessed.

In one or more implementations of the present application, an individual's health information is securely stored as a function of shared and distributed ledger technology to ensure a trustworthy, transparent and unalterable record. For example, at least part of a user's health information is recorded and verified as a function of an algorithm providing a consensus among a plurality of computing devices, such as in a peer-to-peer (P2P) network. Security of the distributed ledger can be provided in a protocol that can include private and public key cryptography. Depending on a respective implementation of the present application, one or more public ledgers can be provided in an open, permission-less blockchain, and/or in a secured permission-based shared or private blockchain, which enables users to define specific access levels for respective parties. Use of such implementations, including in one or more respective blockchains, protects each party's respective health information without incurring risk that such information is not valid or not secure. Thus, in accordance with one or more implementations of the present application, a public/private “hybrid” blockchain model is provided as a function of respective health information, as well as for providing access to the information in accordance with various terms and conditions defined by a respective user.

It is recognized by the inventor that creating secure private objects representing health-related information for validation and storage in a blockchain or other suitable technology, including as collected, processed and/or transmitted by one or more of health devices 105, can be considered to be a source of validated “private” electronic medical records. Control of such private electronic medical records can be maintained by the individual user of information processor 102/user computing device 104. Methods of validating the information as accurate can include confirming a specific identification value, such as a machine access control address (“MAC” address), an internet protocol (“IP”) address, a secured signature (comprising a key, a certificate and/or other trusted mechanism) confirming unaltered and accurate information received via the respective device and/or during a respective data session. In accordance with the present application, such private electronic medical records can be stored in a blockchain or other suitable format and usable in connection with transactions between counterparties in accordance with the teachings herein.

As shown and described herein, a person (i.e. a human being) is provided with information that is stored in secure private objects on the blockchain or similar technology, such as in a validated and securely stored ledger. Information can be collected from various health devices 105 (e.g., digital medical devices), and once validated and securely stored, the person can define sets of rules enabling public access and/or private access to at least specific portions of the blockchain, in accordance with one or more parameters defined by the person.

For example, a user can selectively license or otherwise agree to disseminate validated health-related information securely, such as a function of one or more smart contracts, as known in the art, in which terms and parameters are defined by the owner of the information (i.e., the person) and accepted by one or more parties seeking access to the information (e.g., requesters of the health information). Such requesting parties can desire access to specific health information for, for example, clinical trials, physician assessments, health assessments, health scoring, insurance policies and insurance risk modeling. In connection with DNA sequencing, for example, pharmaceutical companies may desire to research a specific disease, DNA, or the like, and to request access for a specific genome or disease from individuals who know that they are the “owners” of such specific data points. In one or more implementations, access to information in a secure ledger (e.g., a blockchain via Opt-in or other suitable acceptance technology) can be provided in exchange for something of value. Although financial gain is considered as suitable consideration in exchange for access to health-related information stored in a blockchain, the present application envisions an exchange for other forms of value and alternative payment models such as insurance premiums, healthcare visits, treatments and medication, and other healthcare-related value. This is referred to herein, generally, as “the something for something economy.”

In addition to private electronic medical records, such as effected by one or more of the respective devices 105 shown and described herein, institutional-based electronic medical records can be validated and securely stored in a distributed ledger (e.g., a blockchain) in accordance with the features herein. Professionally-generated electronic medical records can be validated as a function of a respective certificate, digital signature or other cryptographic means (e.g., a hash) that represents that the record is authentic, accurate and current. Once added to a blockchain or other suitable technology, the respective medical record is tamper-proof and immune to alteration. This is at least partially supported in a blockchain network, in which a plurality of participating nodes share access to the blockchain and validate transactions and data sources over time.

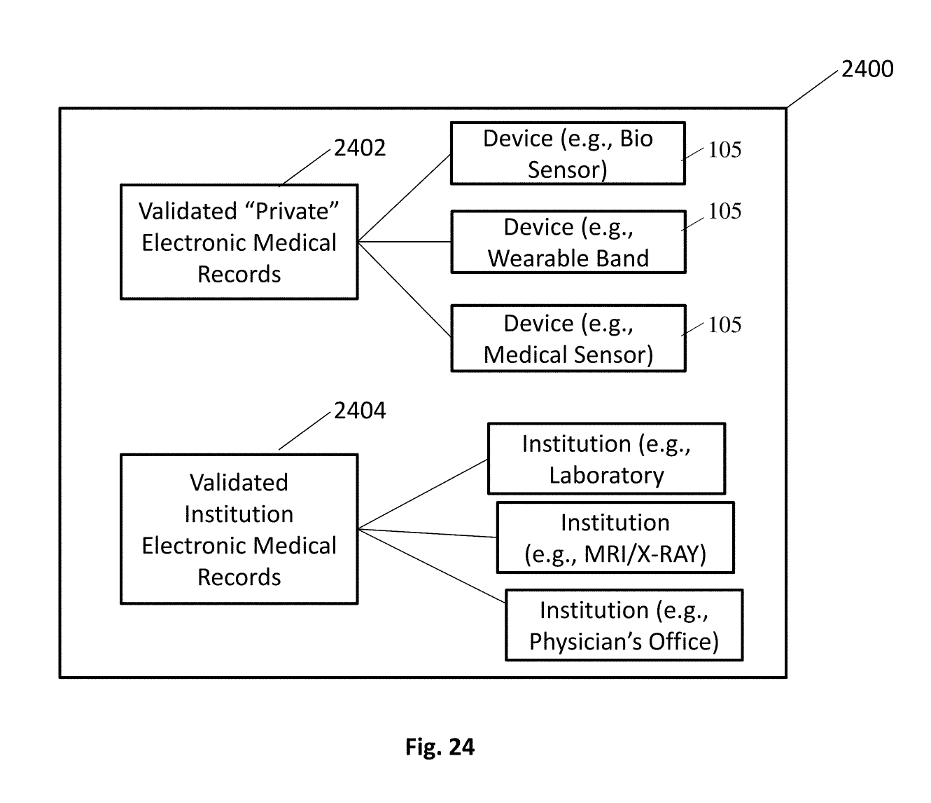

FIG. 24 is a block diagram that illustrates the source of example validated records 2400 that are suitable for entry in a distributed secure ledger, such as a blockchain. In the example shown in FIG. 24, “private” validated electronic medical records 2402 are shown that comprise information received directly or indirectly from various devices 105. In addition, validated and electronic medical records 2404 are illustrated, and include, for example, validated information from a laboratory, from a radiology department (e.g., MRI/X-RAY), and from a physician's office.

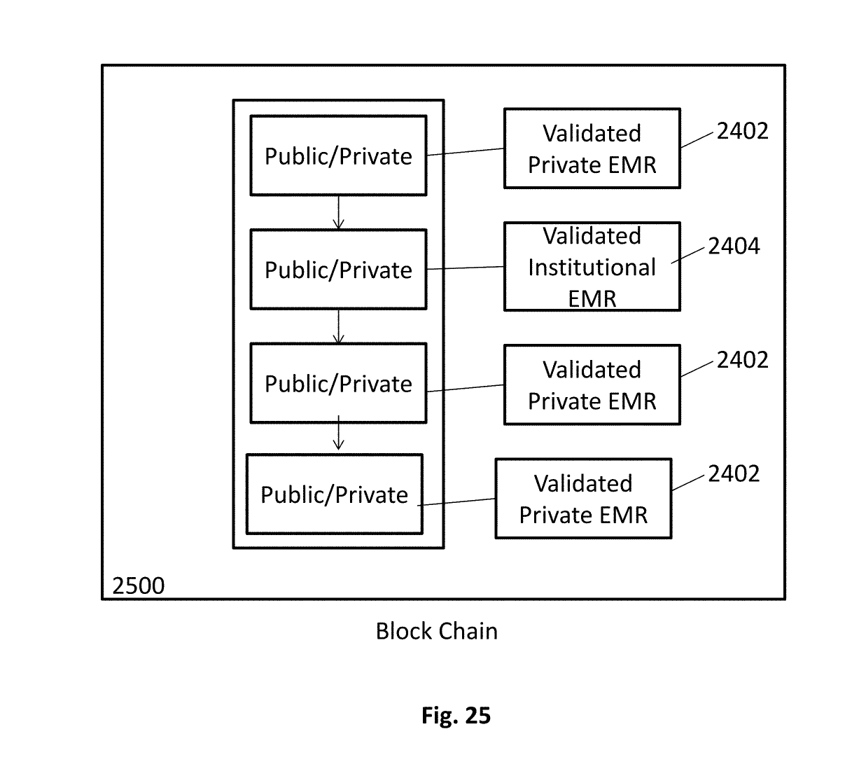

FIG. 25 is a block diagram illustrating an example blockchain 2500 that includes validated private electronic medical records 2402 and validated institutional electronic medical records 2404 which, after securely stored in the blockchain, are secured from tampering or alteration. The entries in the blockchain are, thereafter, usable in connection with one or more transactions, pursuant to terms agreed upon by a user and a counterparty (e.g., a pharmaceutical company seeking research information). In the example illustrated in FIG. 25, some of the validated private electronic medical records 2402 and validated institutional electronic medical records 2404 are defined partially as public and partially as private (illustrated as “Public/Private”). This illustrates further functionality and flexibility of implementations of the present application, in which a user can define particular electronic medical records (or portions thereof) that are securely stored in a blockchain are to be restricted to private access (e.g., are permission-based) and/or those portions that the user elects to be accessible publicly, more generally. This represents the hybrid nature of blockchain technology in various implementations of the present application, and further demonstrates the flexibility of implementing the technology in connection with ownership and access rights to a person's health information, including with regard to an exchange of that information for value.

Moreover, in one or more implementations, individuals can provide access to health information in anonymous fashion, for example, for statistical testing or specific research that the individuals want to support for no specific compensation or gain.

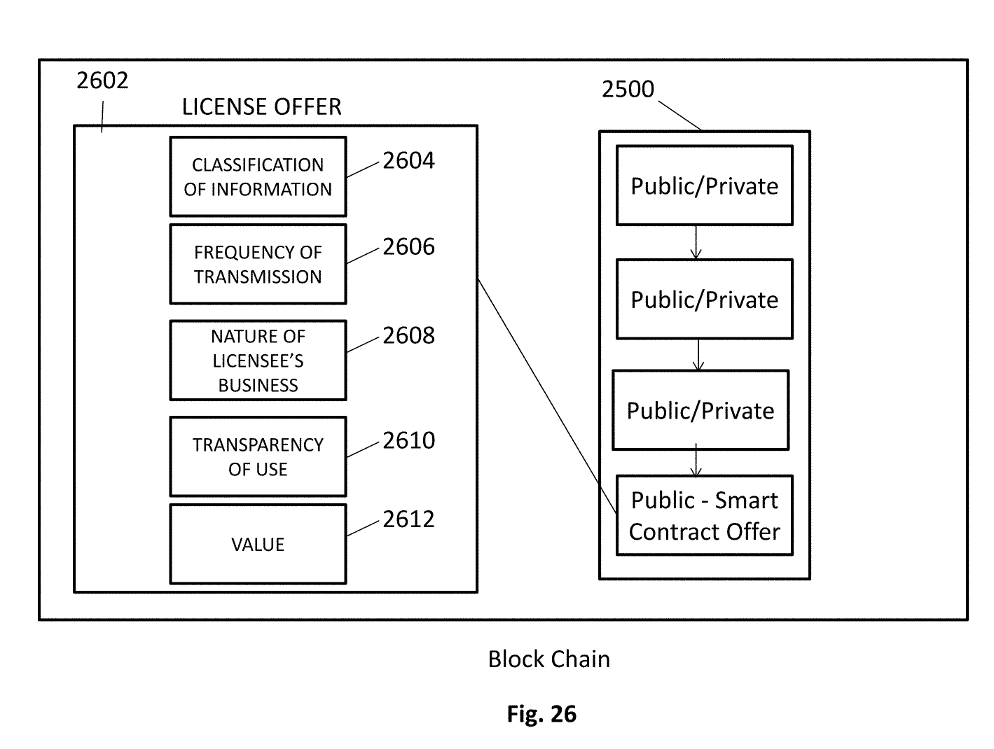

FIG. 26 is a block diagram illustrating an example blockchain that includes a smart contract offer 2602 in accordance with one or more criteria and/or parameters set forth in a license offer of health information by a user. Such parameters, for example, are usable to define the user's terms and can be stored on validating nodes in the blockchain. Upon acceptance of all of the terms by a requesting party, such as a pharmaceutical company, a research institution, an insurance company or other interested party, a transaction can occur pursuant to the terms. In the example illustrated in FIG. 26, a smart contract offer to license the user's health information is validated and securely stored in a blockchain. In the respective blockchain record illustrated in FIG. 26, a plurality of parameters is defined in the offer that represent conditions set forth by the licensor. For example, a parameter 2604 represents a classification of the health information that the licensor is willing to offer for exchange. The health information may be public or private, as set forth in the respective record in the secure ledger, which may impact directly the value the licensor expects to receive in exchange for the information. For example, specific medical information that might be considered to be highly sensitive and private would be considered more valuable by the licensor and, hence, may impact directly the amount of value parameter 2612 in the offer. Other parameters include a frequency of transmission of the respective health information, such as daily, weekly, monthly or some other period. In addition, the example license offer 2602 illustrated in FIG. 26 includes a parameter 2608 representing the nature of the licensee's business. For example, the licensor may not wish to grant a license to health information to a business that is known to engage in offensive business practices. In addition, parameter 2610 is illustrated in example license offer 2602 that requires a transparency of use of the information that is being licensed. For example, the licensor may set forth a term that it wants to know whenever the health information is being used in a specific context. With respect to value 2612, various specifics can be defined, such as that in exchange for the use of the health information, lifestyle information (including, for example, nutrition intake) of the user, discounted or fully paid-for insurance premiums, healthcare visits, medical tests, medication, nutrition or other health-related parameters of value are to be received, including over long periods of time.

Thus, as shown in example license offer 2602 in the respective blockchain 2500, terms and parameters can be defined by owners of health information that are requirements for a smart contract to be made.

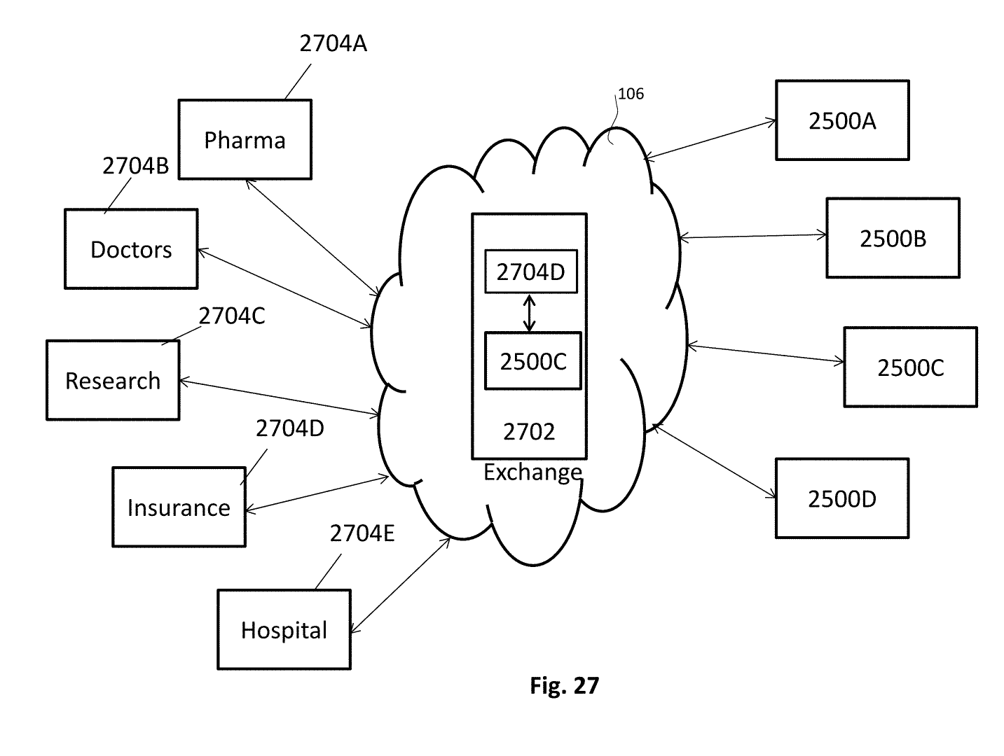

FIG. 27 illustrates an example network of interaction between counterparties and including distributed ledgers that include smart contract offers 2500A, 2500B, 2500C and 2500D from respective individual owners of health information (e.g., license offers for specific health information), and various counterparties who are requesting access to such health information, referred to herein, generally, as requesters. In the example shown in FIG. 27, requesters, such as pharmaceutical companies, doctors, research institutions, insurance companies, and hospitals submit respective blockchains 2704A, 2704B, 2704C, 2704D and 2704E that include parameters defined by the respective counterparties. Of course, other interested potential licensees are envisioned herein, as well. Also included in the example configuration in FIG. 27 is an exchange 2702 in which one of the smart contract offers set forth in blockchain 2500C from an owner of health information can be matched with an offer set forth in blockchain 2704D from a requester of such information. In one or more implementations, in order to update specific ledgers with one or more transactions, each participating node in the blockchain processes the transaction(s) as a function of the terms and logic set forth in the smart contract. Processing transactions in a distributed architecture is more efficient and meets the demands of scalability of the respective participants and ensures trust among the counterparties that the information set forth in the blockchains is accurate, valid and secure.

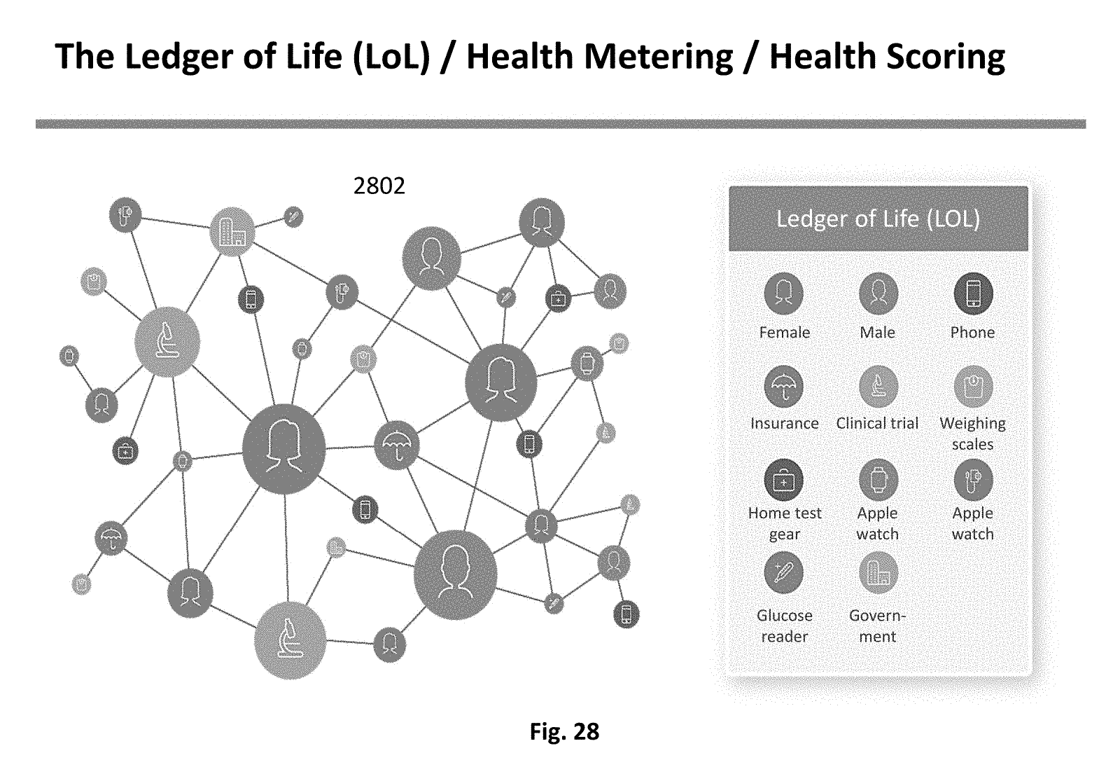

As noted herein, the present application can be implemented in a decentralized architecture, in which validated, secure and distributed ledgers (“ledgers of life”) representing information sent and received from a variety of parties and devices supports a health sharing economy. FIG. 28 illustrates an example decentralized marketplace 2802, including distributed ledgers of life, in accordance with an example implementation of the present application. Smart contracts support a value based, market-priced economy based on health information and health care, in which tangible outcomes are provided. Such outcomes can include, for example, health scores representing multitudes of individuals, as well as specific research and health insurance modeling, in exchange for various value that can include healthcare, treatment, medicine or other sources of value. Implementing an opt-in in an open sharing distributed platform, such as set forth in blockchains, enables records of transactions to be validated, and exchange can be guaranteed over time.

Although many of the examples and discussion set forth herein regard an exchange of value for validated health information that is securely stored and provided in one or more distributed ledgers, the present application supports instances in which access to information via a blockchain or other similar technology without requiring a bargained for exchange, or any further exchange of value. For example, an individual having electronic medical records securely stored in a respective leisure grants access to his or her physician to expedite treatment options. Such access is not necessarily derived from a desire for financial value or other type of gain, but rather to improve access to information and medical care, more generally. Further, this example represents flexibility of the present application to support a one-to-one procedure, without requiring a virtual “exchange” having multiple participants.

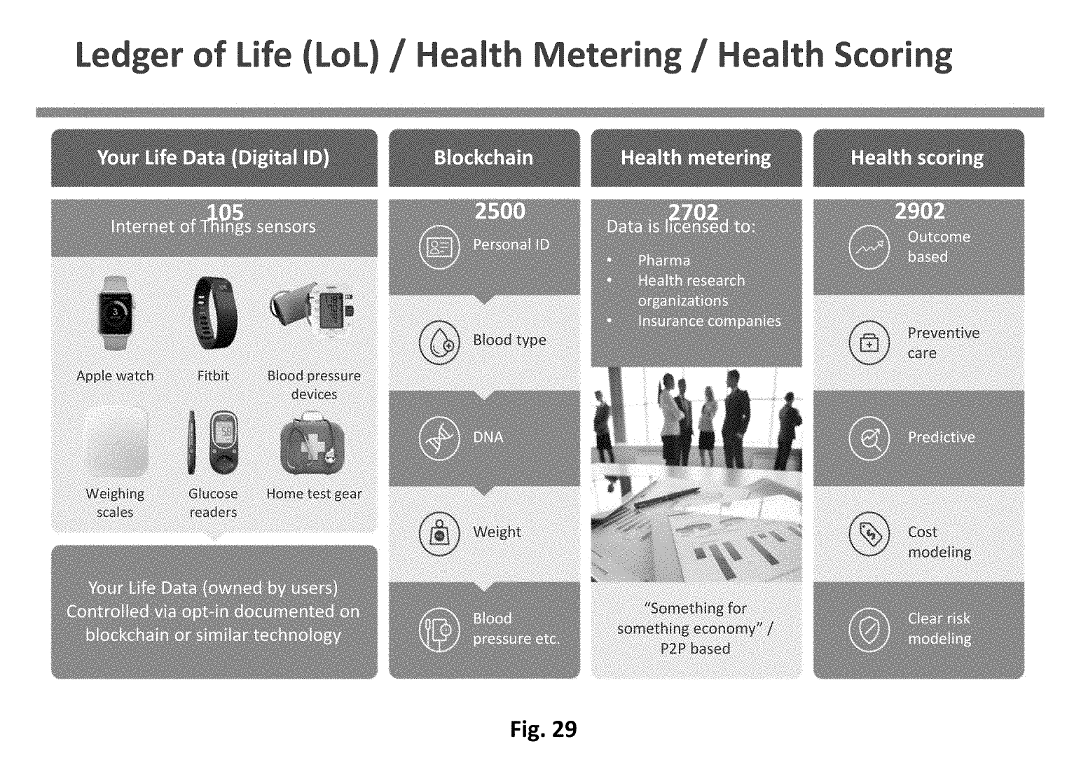

FIG. 29 illustrates an alternative representation of the present application including health information that is generated in distributed as a function of health devices 105 (represented as “Internet of Things”). In addition, blockchain 2500 is illustrated, that includes personal identification information, blood type information, DNA information, weight information, blood pressure information, and other information associated with a user's health.

Example information types that can be included in blockchain 2500 can include:

A user's birth date

A user's sex

A user's age

A user's height

A user's weight

A user's blood pressure

A user's blood type

A user's DNA

A user's Heart Rate

A user's Heart Rate Variability (HRV)

Acid of a user's skin

A user's respiratory rate

A user's skin temperature

A user's blood glycose

A user's blood cholesterol

A user's ECG

A user's steps taken

A user's posture/fat rate/BMI

A user's fall detection

A user's sleep duration/quality/Bed Exit

A user's activity

A user's stress monitor

A user's energy expenditure

A user's daily nutrition intake

It is recognized by the inventor that financial constraints on the insurance industry, including in connection with life insurance and health insurance, particularly as an increasing sector of the population ages. Poor nutrition, for example, is a factor in addition to lifestyle that results in demand for healthcare and treatment and the industry desires improved predictable health outcomes and more accurate risk models. For example, a person is diagnosed with type II diabetes. By providing access to information collected and/or processed by health devices 105, such as via a validated and securely distributed ledger, a respective insurer can provide quality insurance product(s) at highly competitive prices. Since the insurer has access to accurate information representing, for example, that the person is eating well and engaged in an otherwise healthy lifestyle, the likelihood of a claim for treatment is significantly reduced and the insurer can pass savings on to the insured.

Continuing with reference to the example shown in FIG. 29, exchange 2702 includes a match in which health information is licensed to one or more of pharmaceutical companies, health research organizations, and insurance companies (illustrated as “health metering”). Further, outcome information is identified in section 2902, and that includes for example, health score information that is usable for outcome-based studies, preventative care, predictive analysis, cost modeling and clear risk model. FIG. 29 illustrates how respective blockchains (“ledgers of life”) are connected within health metering and health scoring.

In one or more implementations, the present application further supports a donation of medical information, free of charge, such as for not-for-profit research. Such donation may occur after a person passes, in view of a promise or other agreement set forth in a ledger. Example candidates for such agreements may include patients with terminal illness, specific diseases or other health condition.

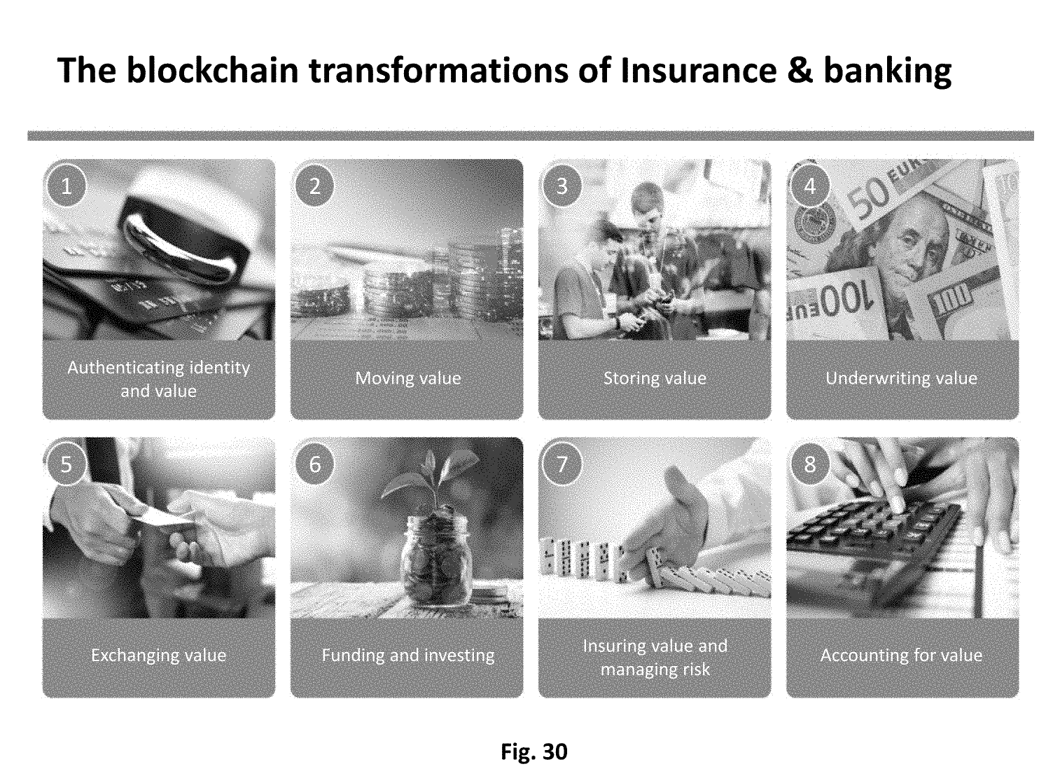

FIG. 30 illustrates example features and benefits of the present application which use of a blockchain serves for transforming various industry, such as insurance and other health related industries. For example, the blockchain or similar technology is operative for authenticating identity and value, moving value (i.e. is transaction-based), stores value (e.g., health information), stores underwriting value, exchanges value, implements funding and investment, ensures value and manages risk, and accounts for value across the industry.

With the exemplary computing system environment being generally shown and discussed above, the method and system of the invention in accordance with illustrated embodiments will now be discussed. It is to be appreciated that the method described herein has been indicated in connection with a flow diagram for facilitating a description of the principal processes of an illustrated embodiment of the invention; however, certain blocks can be invoked in an arbitrary order, such as when the events drive the program flow such as in an object-oriented program. Accordingly, the flow diagram is to be understood as an example flow and that the blocks can be invoked in a different order than as illustrated.

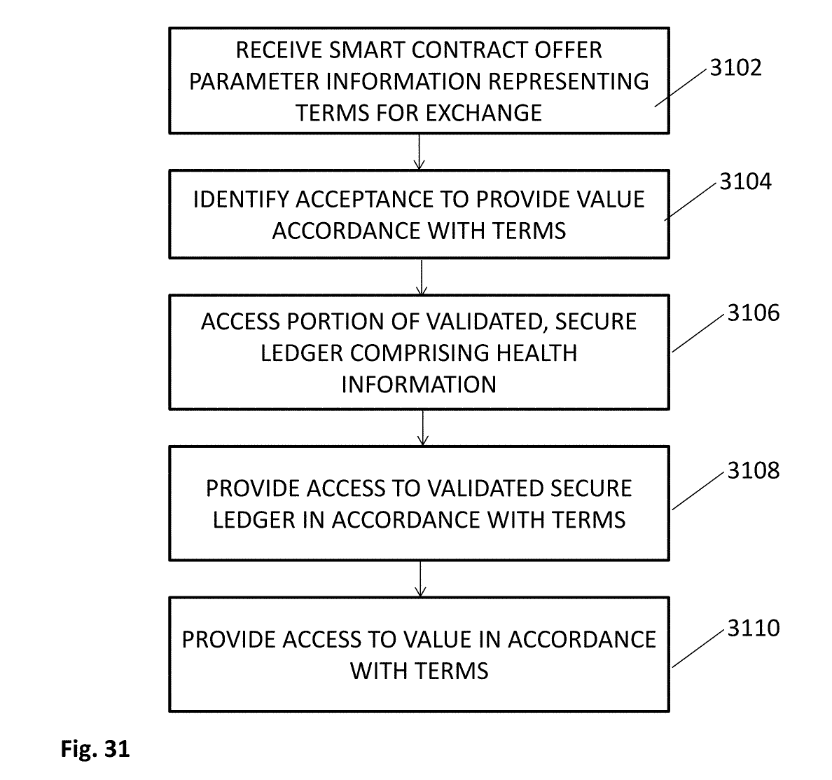

FIG. 31 illustrates a flow diagram of one example implementation of the present application in accordance with exchange 2702 in the implementation shown in FIG. 27. At step 3102, smart contract offer parameter information is received. The parameter information can include, for example, classifications of the type of health information being offered for exchange, a frequency of transmission of the information to a licensee, a preferred classification of a licensee's business and a representation of value in exchange for the health information being licensed. Thereafter, acceptance of the terms and agreement to provide value in accordance with the terms identified (step 3104). A portion of the validated secure ledger (e.g., a blockchain) that comprises health information, is accessed (step 3106). Thereafter, at step 3108, access to the validated secure ledger in accordance with the terms set forth by the parameters is provided. In exchange, access to value in accordance with the terms is provided in step 3110. In one or more alternative implementations, as described herein, such access to health information can be donated or otherwise provided without a requirement for compensation. Thereafter, the process ends.

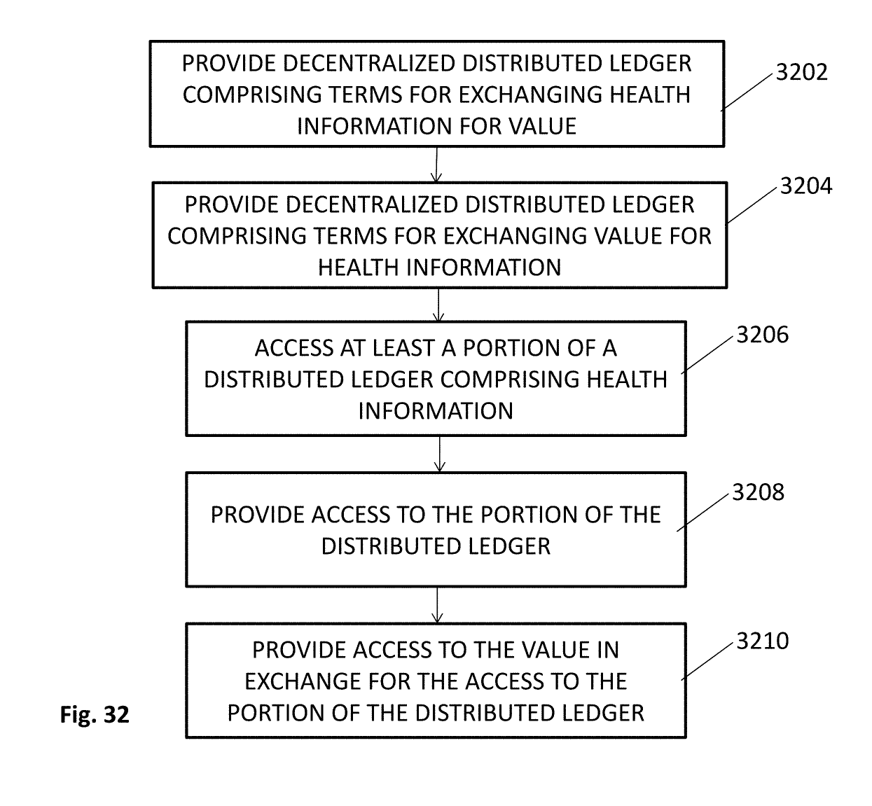

FIG. 32 illustrates a flow diagram of an example alternative implementation of the present application in accordance with at least a decentralized smart contract model. At step 3202, decentralized distributed ledger comprising terms for exchanging health information for value is provided). A decentralized distributed ledger comprising terms are exchanging value for health information is further provided (step 3204). At least a portion of a distributed ledger comprising health information is accessed (step 3206), and access to the portion of the distributed ledger is provided (step 3208). In exchange, value is provided for the access to the portion of the distributed ledger step 3210). Thereafter, the process ends.

Thus, as shown and described herein, a plurality of interactive modules are provided to enable an external party who is in search of certain data points to receive such points as a function of blockchain technology. The centralized ledger network supports a flow of information and operational control from one or more particular points. In addition, computational workload is spread across multiple nodes in the network as a function of a decentralized ledger network that allows nodes to make independent processing and computational decisions. The blockchain network structure of the present application ensures proof of accurate information, accountability and transparency that heretofore was unavailable. In addition, data integrity, data privacy and a newly constructed mechanism for contracting on behalf of a user's private (and public) health information is provided herein.

The present application is now further described with reference to two examples. An individual has access to DNA data and stores the information in a blockchain. The individual decides to store in a blockchain information representing the licensing terms for access to the DNA data, and the terms are set forth in a smart contract. A pharmaceutical company desires access to DNA data, such as offered by the individual, and agrees to the licensing terms set forth in the smart contract offer. In exchange, the individual receives 10 years of free medicine as a function of the data being provided in new groundbreaking medication. In essence, the individual uses the systems and methods disclosed herein to participate in a research and development project, provides access to medical information on the blockchain technology, and is compensated as a function of the blockchain validating the transaction, and receives years of healthcare.

In a second example, an individual has multiple chronic diseases and is deemed to be a high insurance risk. Premiums are extremely high for the individual. Using the systems and methods disclosed herein, individual agrees to participate in a professionally controlled program offered by a life & health insurance company, self-insured companies, retailers, pharmacies or other companies. In exchange for access to the individual's health information, the individual receives a significantly discounted premium for insurance. Access to the individual's health information can include, for example, validated and secured information received via a plurality of bio-sensors, information from physicians and big-data analyses as a function of the laboratory research and testing.

Although the present invention has been described in relation to particular embodiments thereof, many other variations and modifications and other uses will become apparent to those skilled in the art. For example, and in connection with a P2P community, cross jurisdictional and demographic access is available as counterparties are able to assess risk and pricing in respective health insurance communities an opportunity becomes available in view of a free-market exchange.

In addition, the present application can include, in one or more implementations, a decentralized and distributed system in which acquisition and processing of disparate forms of information, such as information associated with health and fitness tracking with information associated with on-line gaming environments, are provided automatically in an integrated hardware and software platform. The present application can provide a computer-implemented system and method configured to acquire health-related and/or medical-related information from one or a plurality of sources, and to calculate a health score based thereon. Information can be processed in accordance with the teachings herein to produce a numerical score as a basis for assessing the relative health of a user (i.e., production of a user's health score). Specific implementations regarding features associated with calculating and distributing a user's health score is more fully shown and described in commonly assigned U.S. Non-Provisional patent application Ser. No. 14/257,855, entitled “AUTOMATED HEALTH DATA ACQUISITION, PROCESSING AND COMMUNICATION SYSTEM” (which is incorporated by reference herein). In one or more implementations, an initial health score can be calculated, such as based on population or other demographic information. In some cases, just a few baseline points of information, such as an individual's height, age, weight, and gender can be processed by a mobile computing device to generate an initial health score. A health score calculated from such baseline information is useful to engage a user quickly and to initiate the systems and methods disclosed herein.

The present application addresses technological shortcomings in order to bridge a gap between disparate sources of information and to utilize the information from such sources in an integrated and holistic fashion. It is recognized by the inventors that, at least initially, individuals often are enthusiastic to use technology to improve and monitor their health progress and keep current with good health practices. Over time, however, maintaining good practice and being current with fitness and health-based technology devices can become a struggle or perceived to be too demanding. Moreover, it is recognized that useful health-related information can be or already is collected in one or more online and/or virtual gaming environments. For example, gaming environments that are implemented or supported by mobile computing devices generate or otherwise have access to information associated with user-activity, such as distances traveled or other fitness-related information. Such information, although often relating to health and fitness, may not be accessed or otherwise available to fitness-based solutions.

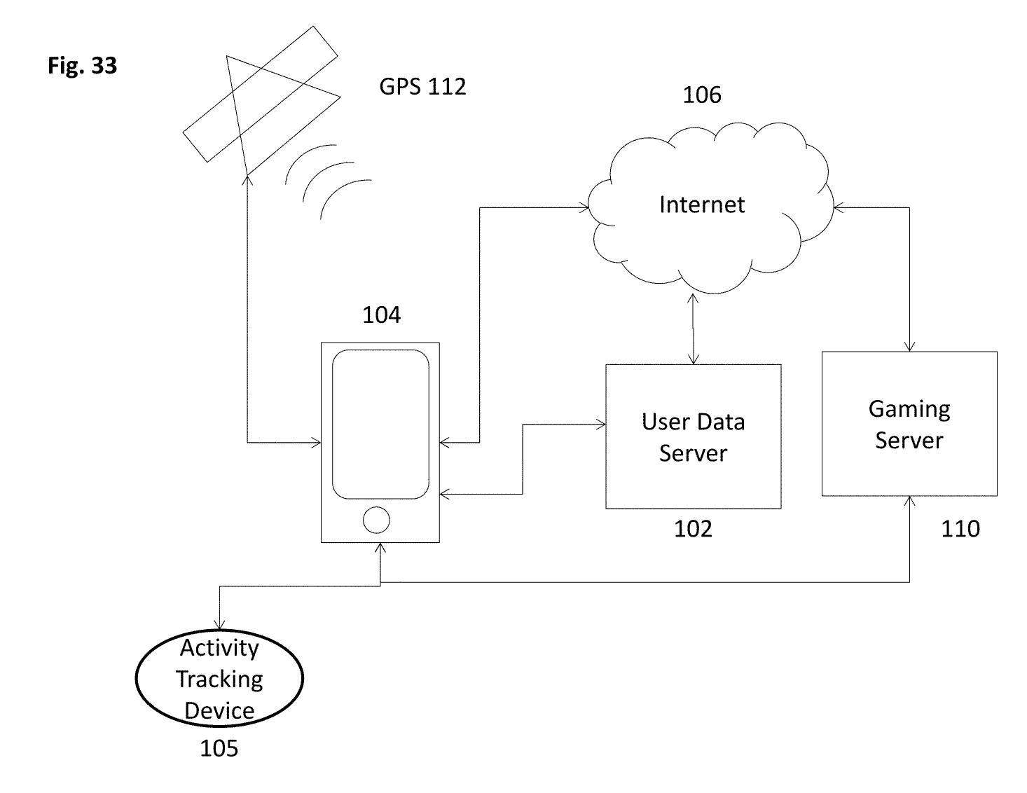

The present application addresses these concerns and provides solutions for tracking, storage and management of health information, including in connection with gaming and other entertainment environments. An example hardware arrangement in accordance with such an example implementation is illustrated in FIG. 33. In one or more implementations, gameplay information is used to augment other activity data, thereby enabling users to keep health information current and accurate. The present application affords this by providing new communication between gaming environments and fitness tracking solutions.

In a particular implementation, a first application operating on a mobile computing device tracks user activity information that is correlated to steps taken by a user over a period of time, such as one day. The first application includes a graphical user interface that operates on the mobile computing device. The mobile computing device communicates with a second application executed by the mobile computing device, and the communication includes information concerning user interaction with the gaming environment provided by the mobile computing device. One or more instruction modules are executed by the mobile computing device that combine, using the first application, the tracked user activity information with at least a portion of the user interaction information into an activity value. The activity value is automatically presented in the graphical user interface of the first application, when the first application is executing.

In an alternative implementation, dynamic tracking of user-based activity is provided as a function of information received from a plurality of data sources. For example, a first application tracks user activity information correlated to steps taken by a user over a period of time. The first application executing on a mobile computing device includes a graphical user interface that presents at least a health score associated with the user. The health score can be provided as a function of baseline information received from a user. Alternatively, the health score can be calculated using a plurality of health activity parameters, such as described in U.S. Non-Provisional patent application Ser. No. 14/257,855. Moreover, communication of gameplay information with a second application executed by the mobile computing device is provided by the mobile computing device, and the tracked user activity information is combined with at least a portion of the user interaction information into an activity value. Using the activity value, the health score of the user is updated and stored in association with the record concerning the user. Moreover, the updated health score is presented in the graphical user interface including by causing a notification independent of launching the first application.

In one or more implementations, the health score and/or the updated health score includes a walking score, which represents, broadly, an inverse of a reduction in all-cause mortality risk associated with walking. Moreover, the walking score can include a buffer referred to herein, generally, as a “health reservoir.” The health reservoir is a numeric value that represents, for example, an ability for a user to rest before his/her health score (e.g., walk score, as described in greater detail herein) begins to decay. Health score decay is described more specifically in U.S. Non-Provisional patent application Ser. No. 14/257,855, such as with regard to a relative weight of an extrinsic lifestyle component or physical activity information. Decay can occur over time with regard to a walk score, in accordance with the present application, and an amount of time or other measurement can be provided by the health reservoir to indicate a buffer before such decay occurs. Moreover, the walking score can include a buffer that allows users to rest for a period of time without loss in their score. The size of this buffer can depend on the accumulated energy earned by a user, for example, from successive walks.

In addition or in the alternative, a reservoir associated with the user's walk score (“waScore”) can translate directly into redeemable value, such as to offset the cost of an item, an insurance premium, or other thing of value. For example, a premium reduction can be calculated by:

$${\sum }_{dayOfYear} ( \frac{waScore}{1000} \$ 0.70 )$$

In one or more implementations, inputs such as tracked footsteps are applied in one or more algorithms that perform a series of steps in connection with calculating a walk score. For example, a daily amount of energy expended by the user throughout a day is calculated. A modulator risk that is based on the risk for all-cause mortality associated with obesity can be further calculated, and an energy amplifier from the obesity risk model can be further calculated. The results are usable to motivate overweight and/or obese users to walk more, such as by providing the values in a user interface and/or to provide encouragement in various ways. Moreover, a value representing net years gained resulting from a positive contribution from energy spent in walking, and, potentially, a negative contribution from excess weight, can be calculated in accordance with the present application.

In addition to the various scores that follow, for example, from the above calculations, the present application can access or otherwise includes a rich gaming environment that is aimed to providing the benefits of inducing users to walk more, as well as the generation of points that can be exchanged for various kinds of rewards. Examples of such gaming are provided herein. In one or more implementations, a gaming environment can be generated and/or customized to implement respective walk scores for particular purposes, such as a discount engine for a health or life insurance company. Examples of pay-as-you-live implementations in connection with insurance are more fully shown and described in commonly assigned International Patent Application WO/2017/091370, entitled “AUTOMATED HEALTH DATA ACQUISITION, PROCESSING AND COMMUNICATION SYSTEM AND METHOD,” which is incorporated by reference in its entirety herein.

In one or more implementations, the present application provides access to a new and challenging gaming environment that can include features, such as quests, with various enticements to maintain user interest. For example, in-game challenges are provided that can include various game levels and corresponding rewards and bonuses upon completion of respective levels. Users can be represented as specific characters and/or with special character names and social interaction is supported for users to have anonymous participation in the levels. For example, a user can choose an alias name that is represented in a shared implementation, such as on a map that is graphically portrayed in a social network site or other socially accessible platform. Furthermore, in one or more implementations, completion of one or more game levels enables users to choose teams that play together, such as in quests and team battles. Further, virtual representations provided on a mobile computing device can be implemented in the real world as well, as quests for gems or other objects of virtual value can be sought after and collected.

In one or more implementations, activity in connection with a gaming environment is measured as a function of points (e.g., “experience points” (“XP”)). At the outset, experience points may be reflected or measured as a function of user activity. For example, one XP can equal one step taken by a user. Over time, however, as users engage in a respective game such as to complete quests or engage in team battles, the number of XP gained can incrementally increase. Moreover, as user's complete levels within a respective game, additional XP can be awarded, which can boost an amount of virtual value amassed by individual users over time. Experience points, in one or more implementations, may be redeemable, such as in connection with lower insurance premiums or other value.

Whether a gaming environment is provided, or information associated with a gaming environment is otherwise accessed, user-based activity such as walking is monitored and information associated therewith is used, such as to update a user's health score, build up a health reservoir, or the like.

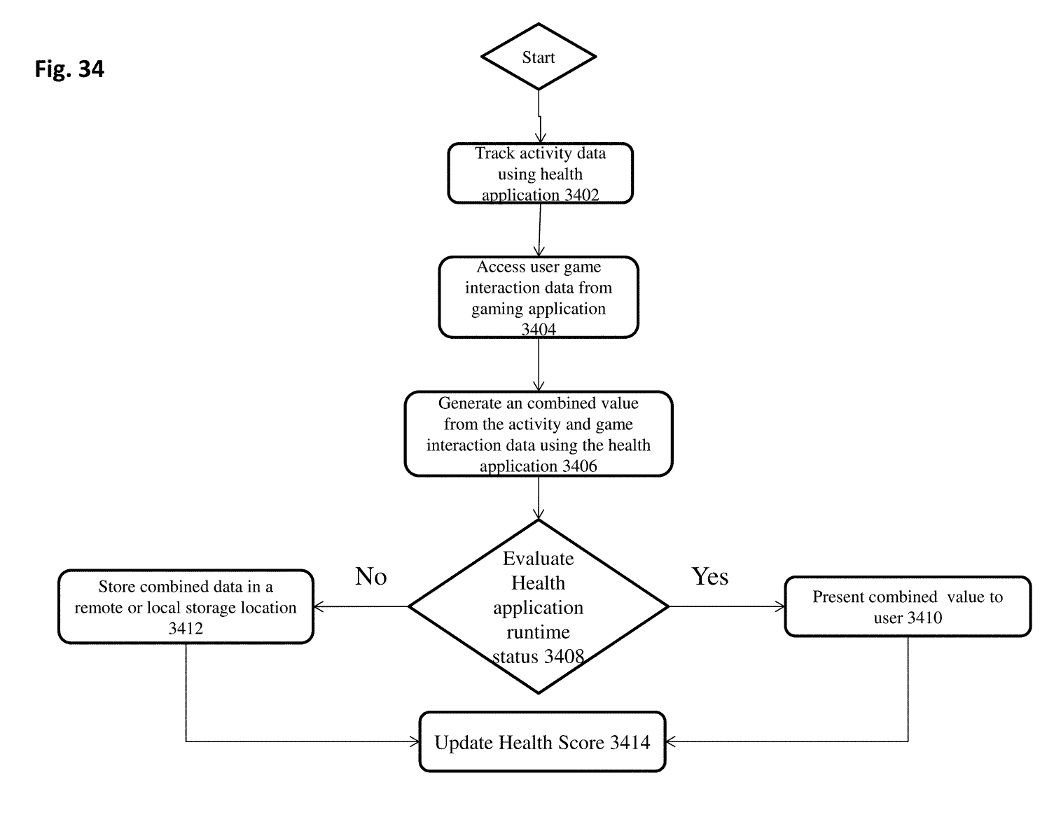

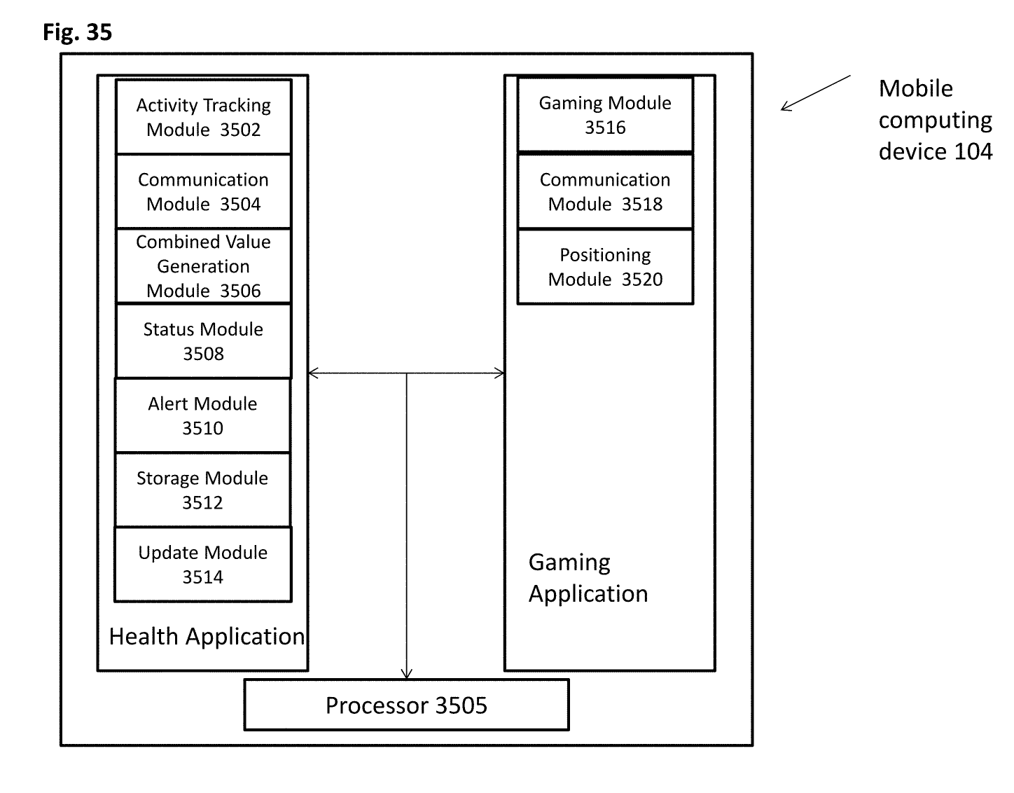

With particular reference to FIG. 33, a dynamic activity data integration system is configured to access user activity data, such as through an activity tracking device. In further implementations, the mobile computing device 102 is configured to access and communicate with activity tracking devices 104, GPS signal transmitters 112, networks such as the Internet 106, and servers, such as user data servers 108 and gaming servers 110, either directly or through one or more network connections. With particular reference to FIGS. 34 and 35, the mobile computing device 104 configured by one or modules of a health application, accesses user activity data as in step 3402. In a particular implementation, an activity tracking module 3502 configures at least one processor of the mobile computing device 104 to access activity tracking data. Here, the activity tracking data may be stored user data, such as prior activity data stored in a database remote or local to the mobile computing device 104. Alternatively, the at least one processor of the mobile computing device 104 is configured by one or more submodules of the activity tracking module 3502 to access from one or more gait or pedometers user data, such as distance traveled over a given period of time.

The mobile computing device 104 is further configured to access data corresponding to user interaction in a gaming application, as in shown in step 3404. Here, at least one processor of the mobile computing device 104 is configured by a communication module 3504 to access data about the present interaction of the user with a gaming application. The communication module 3504 may include one or more submodules configured to access, process, transmit or format data exchanged between the health application and gaming application. The gaming application configures at least one processor of the mobile computing device 104 to access user interaction data during the excitation of the gaming module 3516. Here, the gaming module 3516 configures at least one processor of the mobile computing device 104 to initiate a series of activities incorporating user interactions. In a further implementation, acquiring interaction data includes accessing one or more GPS geolocation measurements from a GPS satellite using positioning module 3520. Such gathered data is transmitted back to the health application using the communication module 3518.

It is recognized by the inventors that information collected by a pedometer or other tracking device may be duplicative of information already accounted for in connection with a gaming environment. Accordingly and to preclude double counting of user activity, the present application precludes either information collected from a tracking device or from a gaming environment when duplicative information is detected. Examples of precluding double counting of information are also shown and described in commonly assigned International Patent Application WO/2017/091370.

Upon acquisition of the gaming state data using communication module 3504, at least one processor of the mobile computing device 104 is configured to combine the activity tracking data and the received interaction data. As shown in step 3406, at least one processor of the mobile computing device 104 is configured by a combined generation module 3506 to generate a combined value from the activity and interaction data. The communication module may include one or more submodules configured to access, process, transmit or format data in relation to the step described.

A status module 3508 configures at least one processor of the mobile computing device 104 to evaluate the current state of the health application. For instance, as shown in step 3408, the run time of the health is evaluated.

Where the health application is currently executing, the combined score generated in step 3408 is presented to the user via one or more visual, audio, tactile, or combined notification protocol.

As shown in step 3410, the combined value is presented to the user. In a particular implementation, at least one processor of the mobile computing device 104 is configured by an alert module 3510. Here, the alert module causes one or more graphical user interface elements of the mobile computing device 104 to generate a text, or iconographic depiction of the combined value.

The alert module 3510 may include one or more submodules configured to access, process, transmit or format data exchanged between the health application and one or more system level or application level GUI applications.

Where the health application is not currently executing, the combined score generated in step 3408 is stored in one or more remote or local storage locations, such as a database. As shown in step 3412, at least one processor of the mobile computing device 104 is configured by a storage module 3512 to store the combined value, and other user data in a user profile or account.

In one or more further implementations, a health score of the user is updated using the tracked data using the activity value as in step 3414. The mobile computing device 104, configured by an update module 3514, accesses one or more user profile dataset containing health score data and updates the health score based on at least the activity value tracked in step 3402.

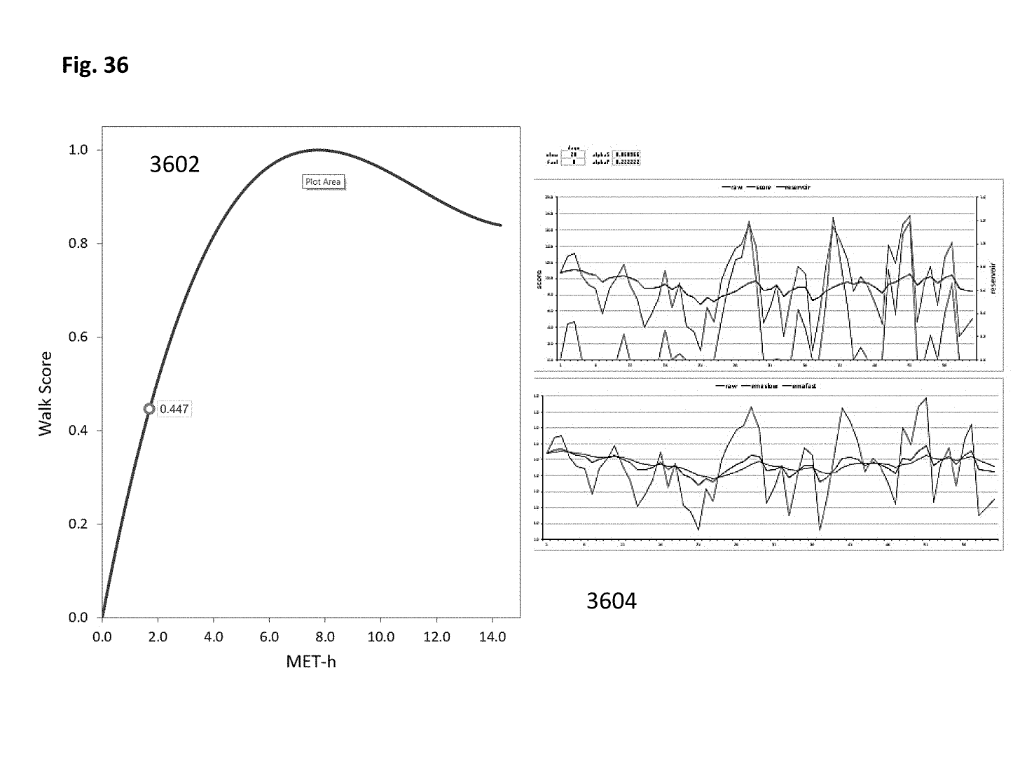

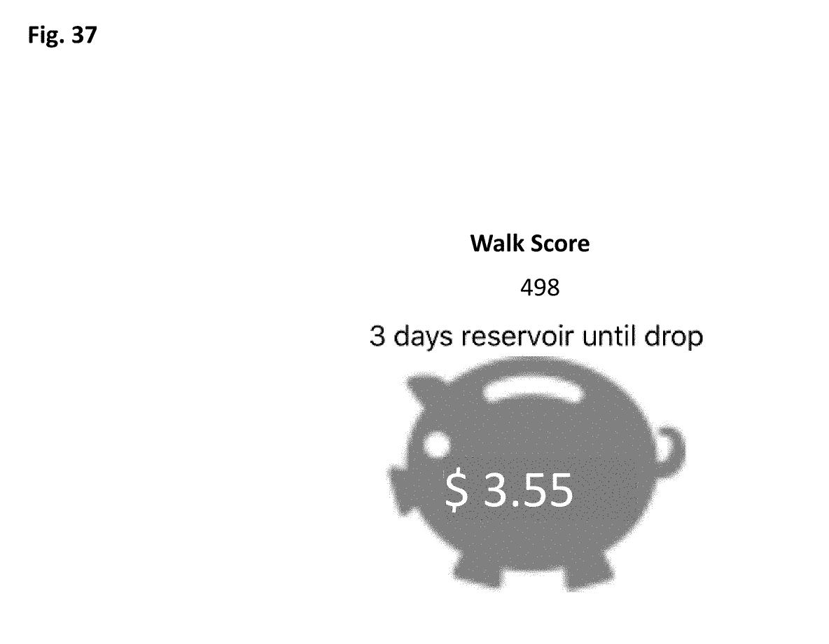

FIG. 36 illustrates graphs associated with a calculated walk score, in connection with the present application. In graph 3602, for example the plot area is provided that identifies along the Y-axis a walk score and the corresponding metabolic equivalent value along the X axis. Graphs 3604 illustrates the comparison of raw information, and information stabilized with one or more filters, such as such as an exponential moving average (“EMA”), as well as the reservoir value, in connection with a walking score. FIG. 37 illustrates an example walk score value (498) a corresponding number of days until the walk score decays or drops, and a corresponding cash value ($3.55) that can be applied, such as to offset an insurance premium.

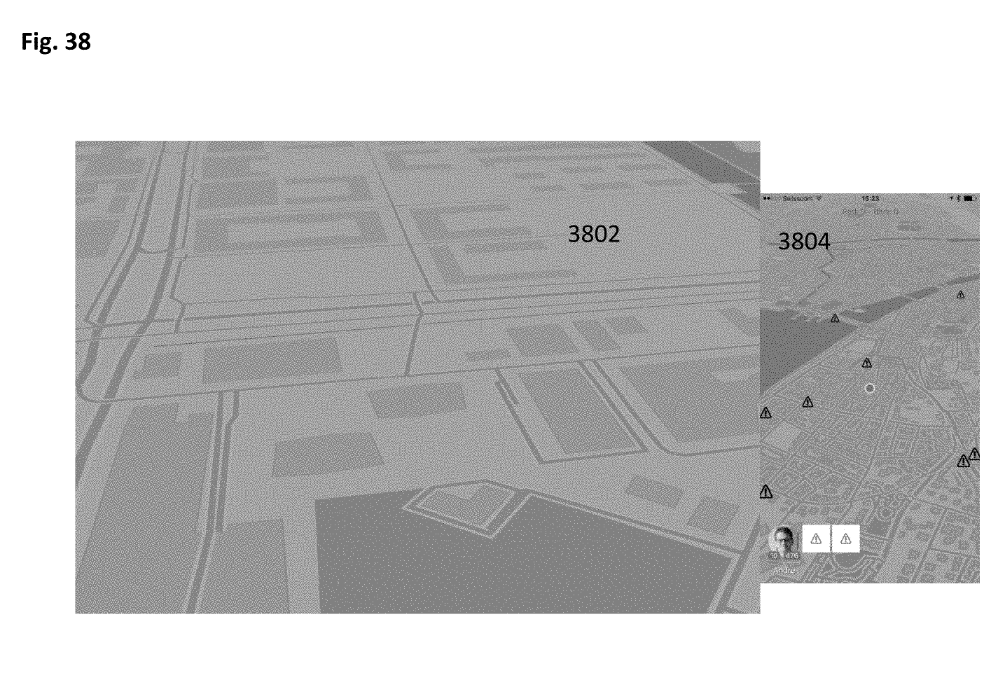

FIG. 38 illustrates an example implementation that includes a graphical virtual map 3802 that virtually represents locations in the real world and corresponding map portion 3804 includes respective icons where points of interest (e.g. items in a quest) are located as well as where a respective player may be positioned.

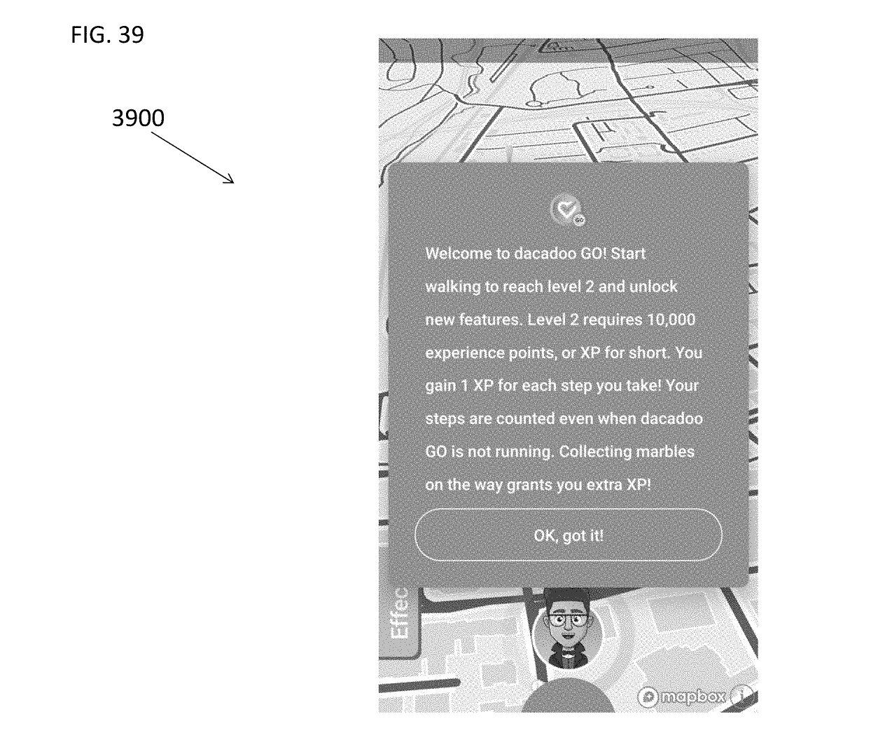

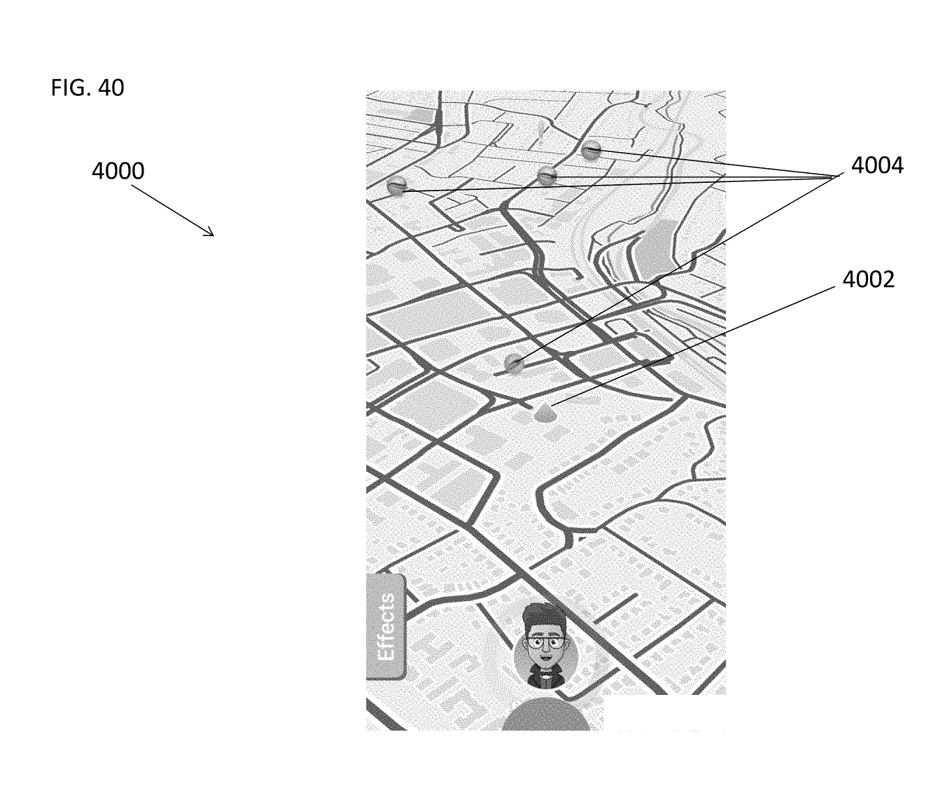

FIGS. 39-44 illustrate example screen displays of a game platform in accordance with an implementation of the present application. FIG. 39, for example, shows display screen 3900 provided on a user computing device that is configured with a mobile software application (“mobile app”) in accordance with the example implementation. FIG. 40 illustrates an example display screen 4000 that includes a map, a representation of a player, a directional icon 4002, and gems 4004. Directional icon 4002, for example, provides a graphical representation of a direction that a player is headed vis-à-vis a map. In one or more implementations, a player can, by a touchscreen interface, adjust the view of the map as a function of direction that the player is headed. The ability to zoom in and out of views of a map, however, can be restricted such that a player's viewpoint with regard to a map is constrained to a respective predefined radius, for example, by the directional icon 4002. Gems 4004 represents virtual and/or physical items that a player navigates to and obtains as a function of gameplay. For example, gems 4004 appear on the map, and a player collects them by walking to the gem. In one or more implementations, as gems 4004 are collected, XP are accumulated.

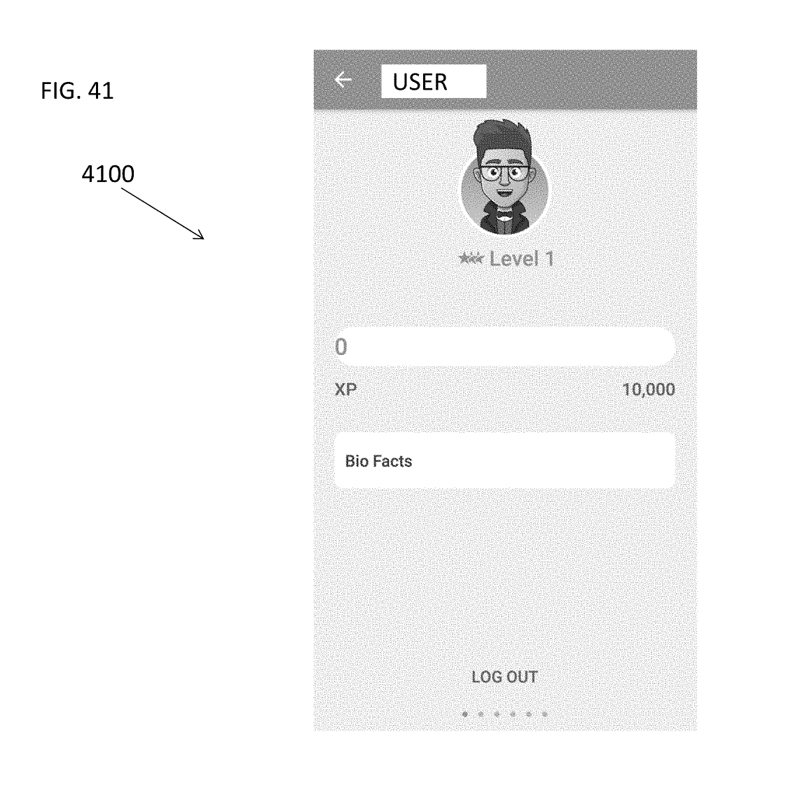

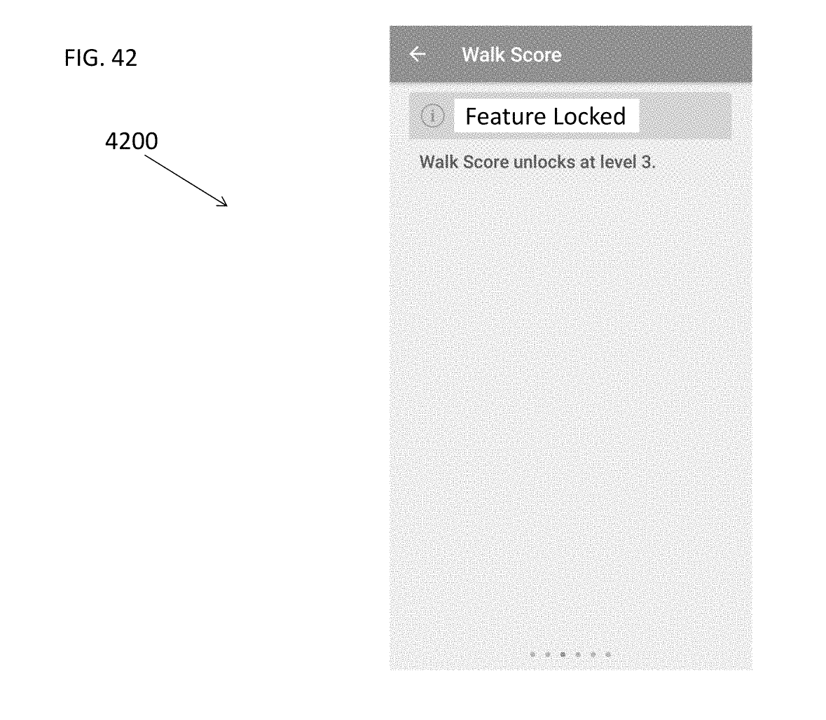

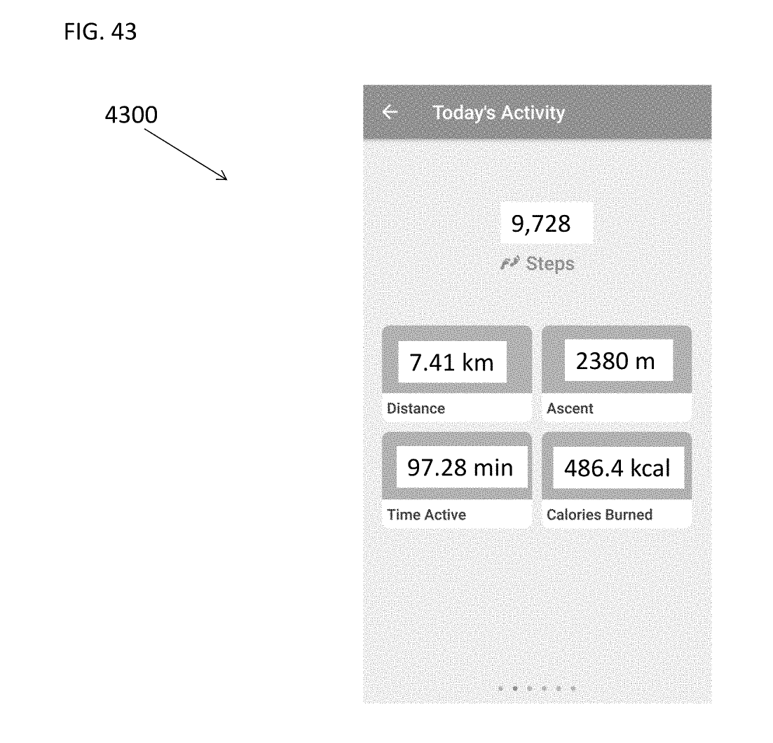

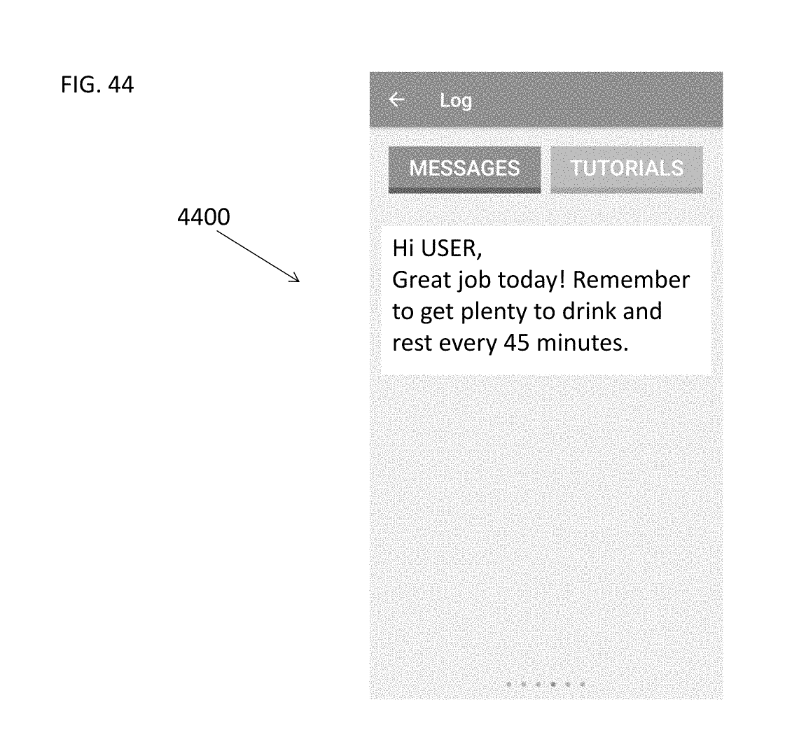

FIG. 41 illustrates an example display screen 4100 that represents a respective level that a user is on, a number of experience points accumulated, as well as various bio facts that may be pertinent, such as heartbeat, respirations, temperature, or the like. FIG. 42 illustrates an example display screen 4200 that identifies a feature (e.g., the “walk score” feature) that is currently locked, an interactive message display that identifies a particular level in which that future may become unlocked. FIG. 43 illustrates an example display screen 1000 that identifies activity for a given day. As illustrated in display screen 1000, total number of steps, distance, ascent, amount of time of activity, and calories burned is provided. FIG. 44 illustrates an example display screen 4400 that provides messaging and/or tutorials. In the example illustrated in display screen 4400, the user is encouraged and reminded to remain hydrated and rested.

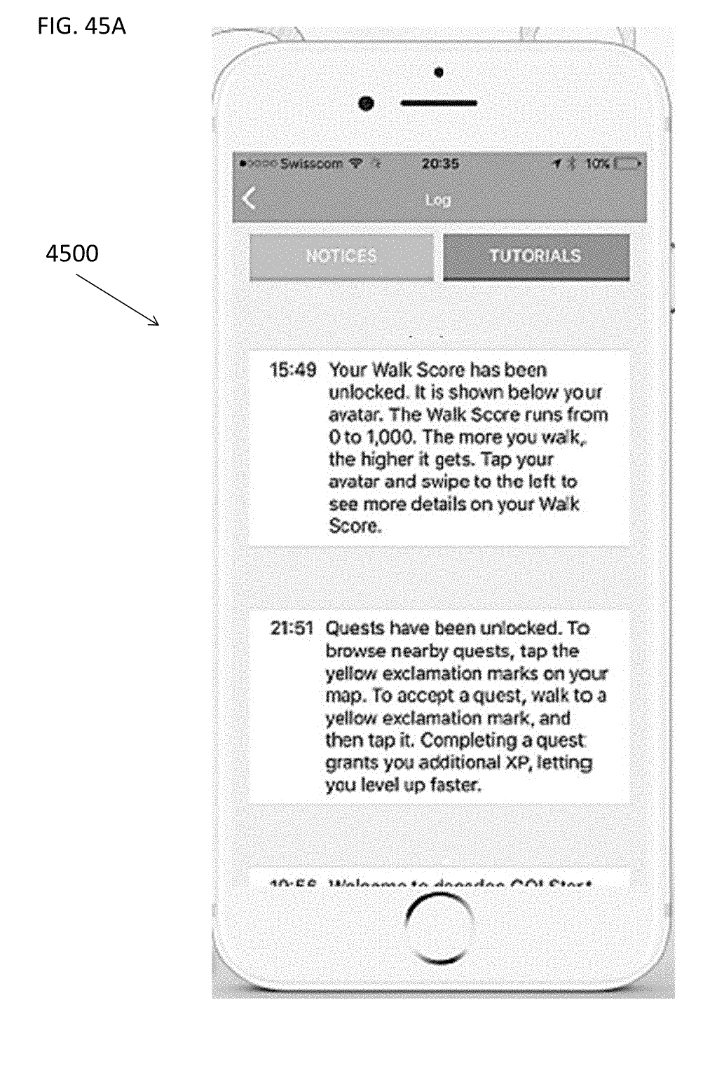

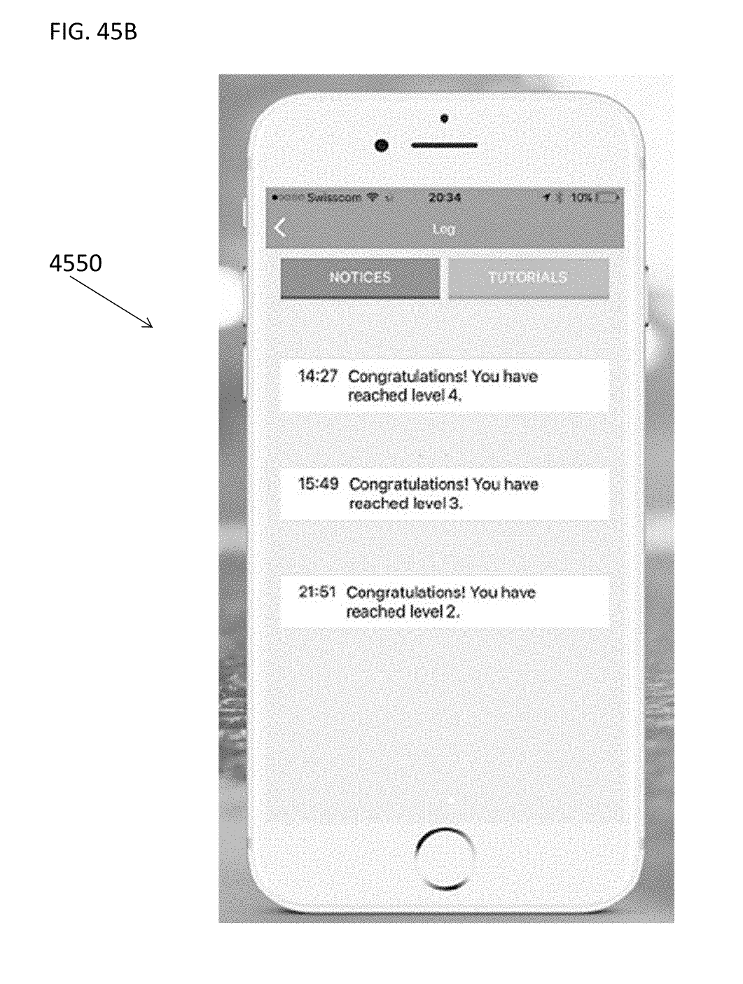

FIGS. 45A and 45B illustrate example display screens 4500 and 4550, respectively, that identify to a user the levels that the user has reached and/or unlocked, as a function of experience points, activity, or other suitable criteria in accordance with one or more implementations.

In one or more implementations, team play is supported in which a plurality of users can work together in furtherance of a single team (e.g., a “blue” team or a “red” team). Team play can be provided by unlocking a respective level, following completion of one or more previous levels. Choosing a team allows players to battle for daily supremacy by collecting gems 4004 that appear on the map for a respective team. In one or more implementations, players walk to the gems 4004 to pick them up. All active players in the winning team gain additional experience points once the competition ends. The present application supports a form of a social game, that emphasizes teamwork and collaboration.

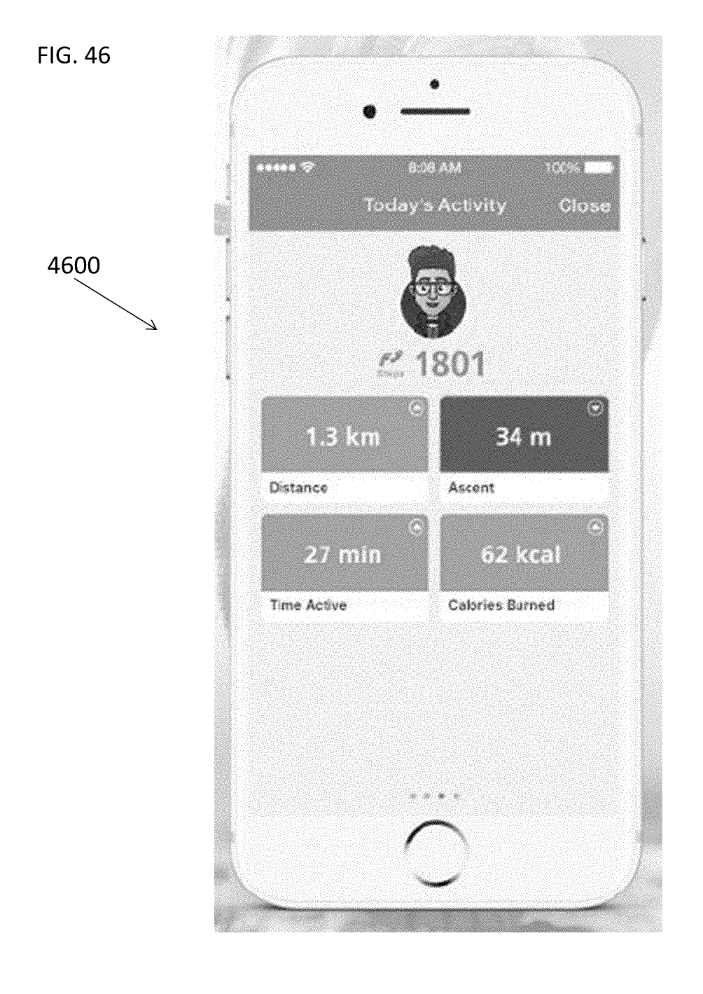

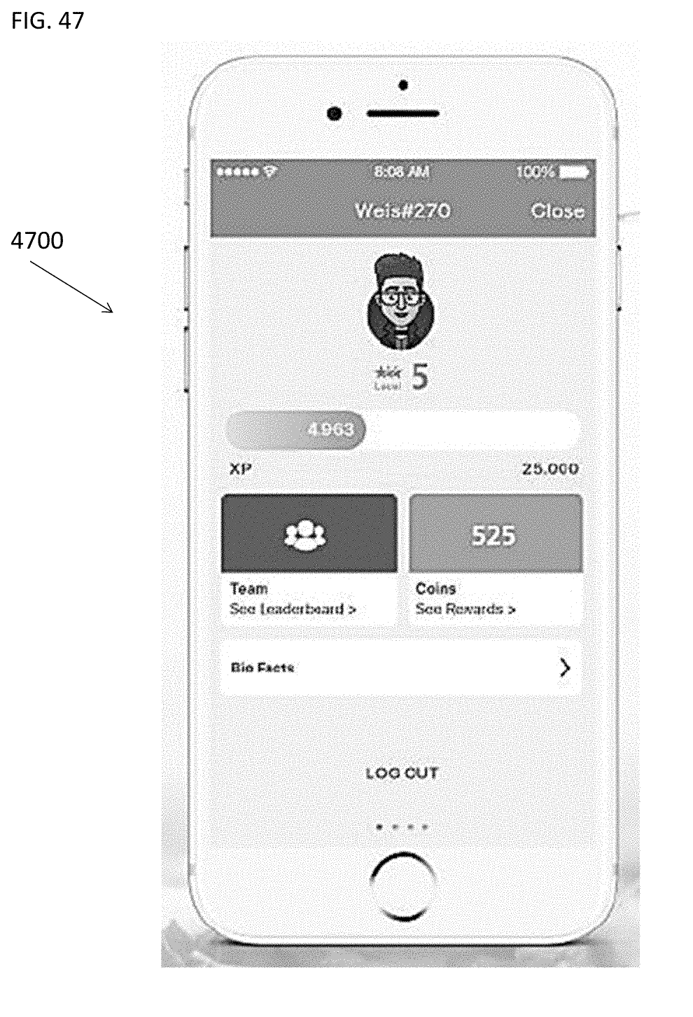

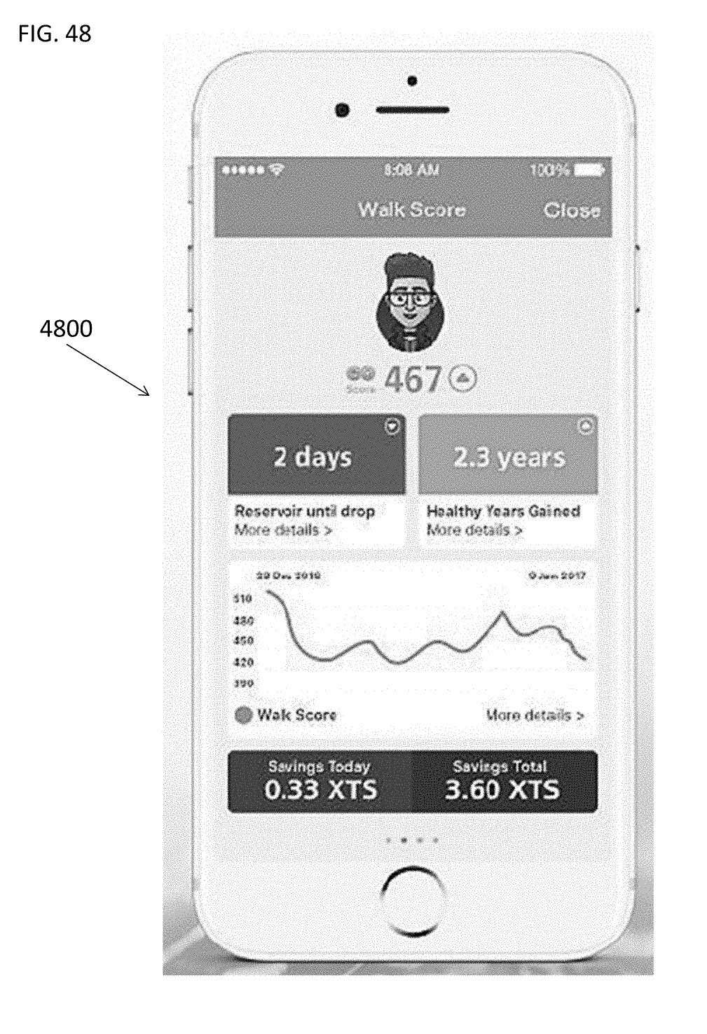

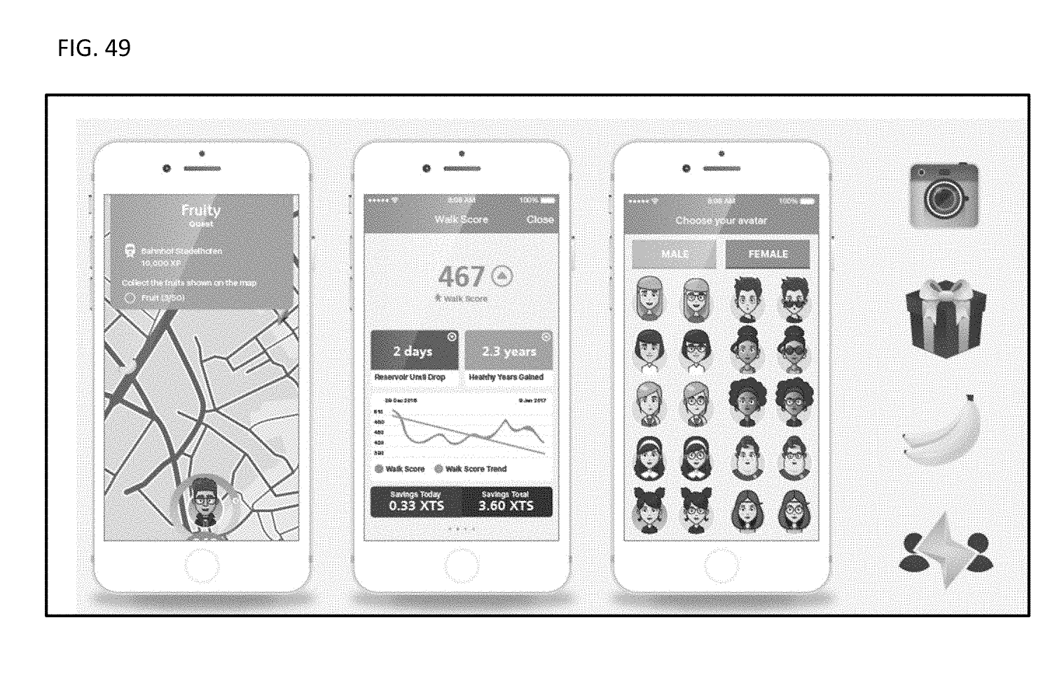

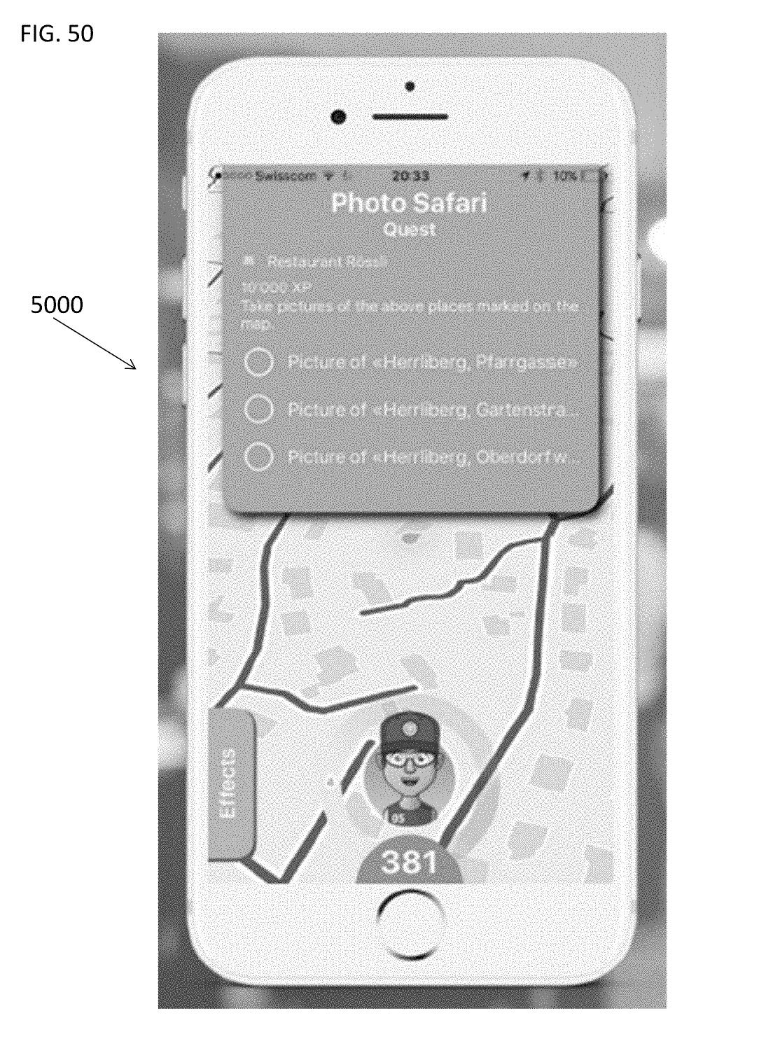

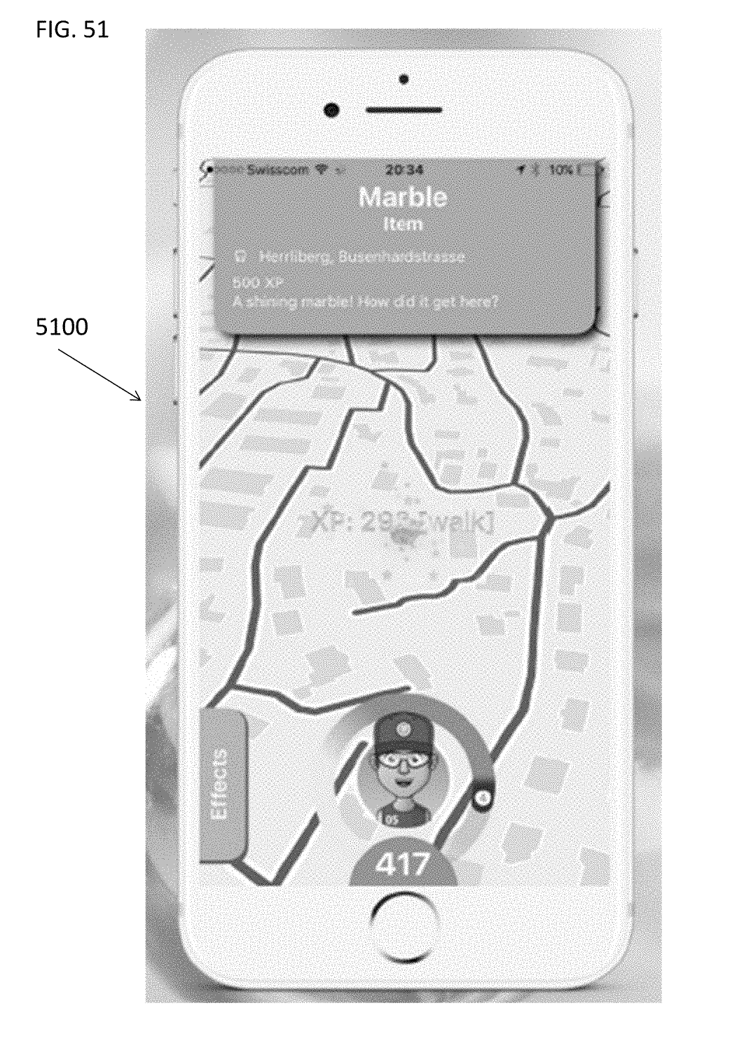

FIGS. 47-51 illustrate example display screens that provide information associated with activity, accomplishments, and status over time, in accordance with one or more implementations of the present application. For example, activity information is illustrated in display screen 4600 (FIG. 46). Further, quantities of experience points, accumulated coins, and a representation of a player's team is provided via display screen 4700 (FIG. 47). Moreover, an amount of time remaining in a reservoir and an amount estimated time gained as a function of a healthy lifestyle is represented in display screen 4800 (FIG. 48). Display screen 4800 can also include a line chart representing fluctuations in the user's calculated walk score over time. FIG. 49 illustrates additional features of one or more implementations of the present application. For example, fruit is illustrated as a form of gem 4004, and various avatars are illustrated for a user to select to represent himself or herself. Gems 4004 and functional icons are illustrated as a camera, a present, fruit and jewels/gems.

In one or more implementations, the present application provides a quest configured as a photo safari. For example, an image can be taken by a user's computing device 102, and transmitted to, for example, the gaming server 110 for confirmation that a user has reached a respective location. In one or more implementations, one or more processors are configured by executing code that enable the processor(s) to distinguish “correct” pictures, for example, of a building, from “wrong” pictures, such as taking a picture of the sky or the ground. This can be implemented as a function of a machine learning model that uses color histograms and gabor textures, including for image classification and similarity searching. This provides functionality to confirm a user's presence at a respective location and time.

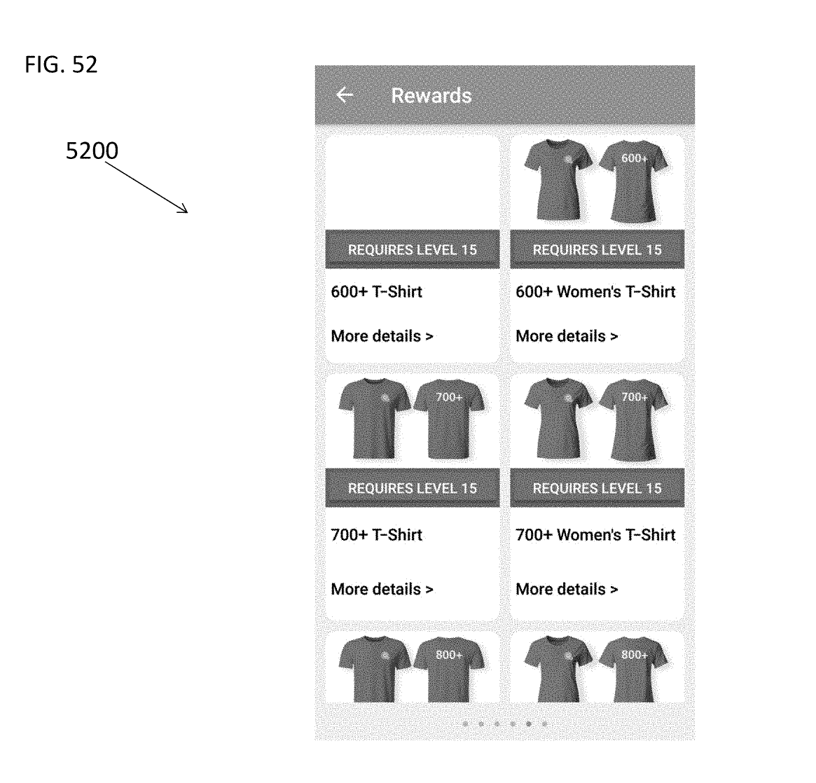

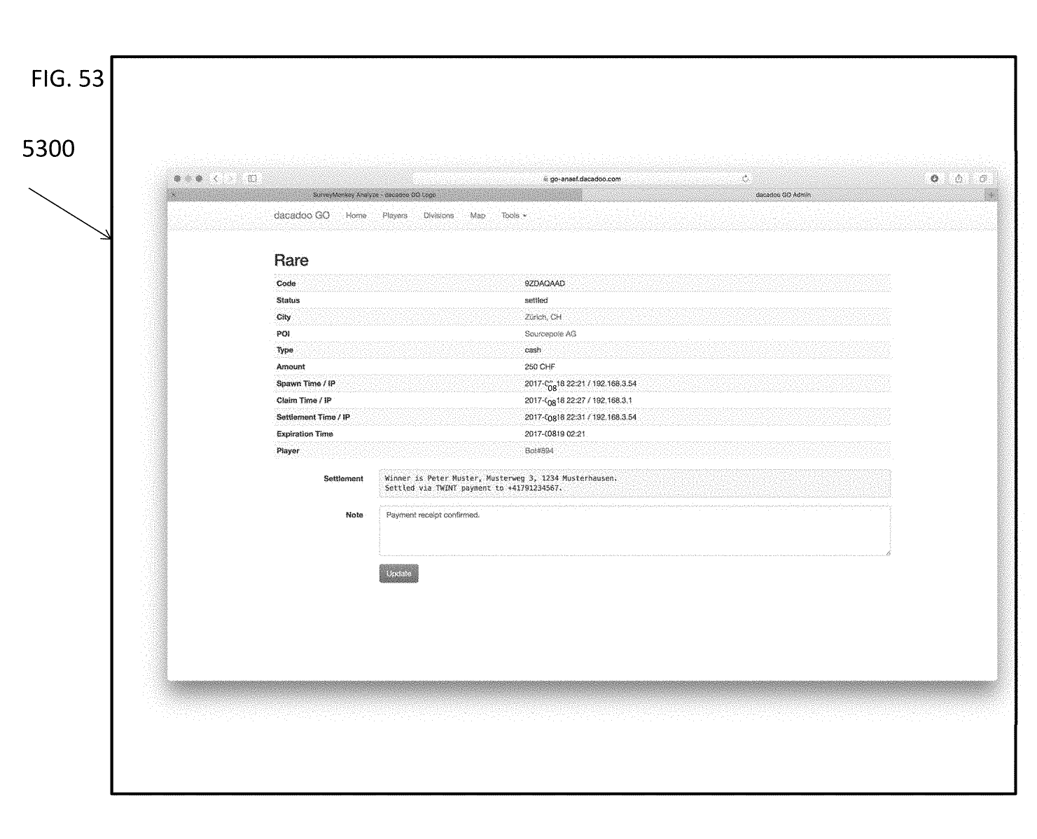

FIGS. 52 and 53 illustrate an example implementation of a photo Safari, in connection with the present application.

In one or more implementations, a system of rewards is provided at a high (e.g., the highest) level, and in connection with a minimum number of experience points that have been gained from “Quests” (e.g., completing various levels and amassing various gems. In one or more implementations, “coins” can be earned and, thereafter, redeemed in a reward shop. Partners can fully customize the content of the reward shop with their own reward merchandise. Providing a reward system in such a fashion provides long-term motivation players. FIG. 52 illustrates an example screen display 5200 of an on-line reward shop, in which users can redeem coins for goods/services.

Thus, in one or more implementations, the present application provides an online environment where users can “find the money,” in which value is amassed that is redeemable for money payments. For example, upon collecting gems and other spawns representing money, a user can connect to gaming server 110 to redeem his/her payments, and money can be transferred to the user, such as via known payment applications.

In addition, or in the alternative, spawns, gems or other objects can be redeemed for physical and/or virtual items of value. In one or more implementations, value can be obtained by users playing the game, which is thereafter redeemed for healthcare, such as insurance premiums, deductibles, medicines or the like.

In one or more implementations, the present application supports providing prompts representing one or more themes which are selectable for a user to represent that he or she has “visited” the specific gem spot or location. Moreover, one or more “pop-up” functions are supported for one or more parties, acting as sponsors for users to “Find the Money,” to gain visibility, traction and fame. For example, user traffic can be driven to specific locations, such as a restaurant with empty tables, a disco not filling up that night, or the like. The present application provides a new form of a power social traffic walking map, which generates revenue while simultaneously growing businesses. In one or more implementations, the sponsors can work together, such as in the form of a competition or a collaborative venture.

In one or more implementations of the present application, “partners” purchase advertising/placement that is represented in the mobile app while a user is playing the game. In one or more other implementations, a business to business to consumer (“B2B2C”) model is supported, in which a company licenses rights to the mobile app and configure the app with custom branding.

In one or more implementations, data management and functionality is provided via one or more processors specially configured by executing code. For example, a user who interacts with the platform (via mobile app or web app) and triggers a server call (e.g. tracks, quests, etc.), can be classified as an “active user.” A daily active user (“DAU”) can include a user having a mean value of per-day active users in the past 30 days. Monthly active users (“MAU”) can include total active users within a monthly period, such as within past 30 days. With regard to engagement, values associated with active users can be expressed as a percentage, and can represent, for example, a degree consistent use, such as “stickiness” of the application.

It is recognized that there is a potential for cheating, particularly as users compete for value. In one or more implementations, a processor configured by executing code monitors a user's heart rate, which can indicate whether a user is actively participating.

In one or more implementations, an administrator user can spawn a rare reward in a given city. The administrator can configure a specific amount and currency, and a logging function occurs to record the time and IP address of the action. Referred to herein, generally, as “spawns,” objects that a player can interact with by walking to them, such as quests and gems, or the rare spawns are supported by the present application. A rare spawn can be assigned a specific status, such as “spawned.” In one or more implementations, a precise location of the rare spawn is redacted, i.e. not shown. Instead, the administrator (or associated parties) is free to promote the location of the spawn, such as via social media.

Continuing with this example, one player may be entitled to claim the spawn, by physically walking to it and selecting a representation of the spawn on a map displayed on the player's user computing device. The player can then receive a notification, such as a “win code” (e.g., 8 letters and digits) and can receive a message associated therewith. In one or more implementations, the player's ID, time and IP address are logged and a day trace can be generated for software robot (“bot”) detection. Provided it is determined that the activity is not performed by a bot, then the spawn is claimed, and the status of the rare spawn is revised from “spawned” to “claimed”.

In one or more implementations, a delay occurs until the rare spawn despawns on other players' maps. These players can receive a notification that the specific spawn has already been claimed.

An administrator can browse and process claimed rare spawns. Upon spawns being claimed, the respective locations thereof may no longer be redacted. For example, the administrator may be authorized to view information about the claiming player, can browse the claiming player's record, and can also view a day trace that was automatically generated to detect bots. The administrator can further be authorized to settle the spawn, for example by reaching out to the player, and requesting name, address, and mobile phone number. Thereafter, the administrator can, for example, execute a TWINT payment to the mobile phone number of the claiming player. The administrator can enter the complete settlement information into the respective data fields and, thereafter, settles the rare spawn. Furthermore, the precise time and IP address of the settlement action can be logged. Once this occurs, the settlement information can no longer be changed. The rare spawn enter status “settled.” An example data record illustrating a settled spawn is shown in the example display screen 5300 in FIG. 20.

Alternatively, in case a rare spawn is not claimed, then the spawn may enter a status of “expired,” such as after ˜4 hours. Once expired, a spawn may no longer be claimed.

Thus, as shown and described herein, an actual financial transaction is occurring. Accordingly, accountability with timestamping and logging of IP addresses is therefore in place, as is automated bot detection.

In one or more implementations, the game server 110 manages so called spawns on the world map. A common operation when determining the game state of a player is to query the database for spawns in a circular radius around the player. In order to conserve computing resources, including by reducing CPU time, one or more implementations of the present application employs functionality to cache spawns by quadrants in a memory cache (memcached). The cache is filled from a database (e.g., MongoDB), when information for a quadrant is missing. In the event that spawns of a player are needed, a query for a respective quadrant where the player is located, as well as the 8 surrounding quadrants, is executed. Thereafter, a distance calculation is performed for each spawn, and only those that are within a desired radius are used for entry into an effective result set. This optimized approach reduces load on a test system by factor ˜30.

As noted herein, a machine-learning model is employed to detect the use of bots, which would violate gameplay. In one or more implementations, the model factors the relationship and distribution of various data points, such as player velocity between locations, GPS accuracy, tap distance to markers, country from IP address vs. country from geolocation, distance between locations, steps, number of locations, number of interactions, active hours, and other suitable data elements. By factoring these data elements and confirming the accuracy and relevance of each, the likelihood of the system getting spoofed by bots is significantly less.

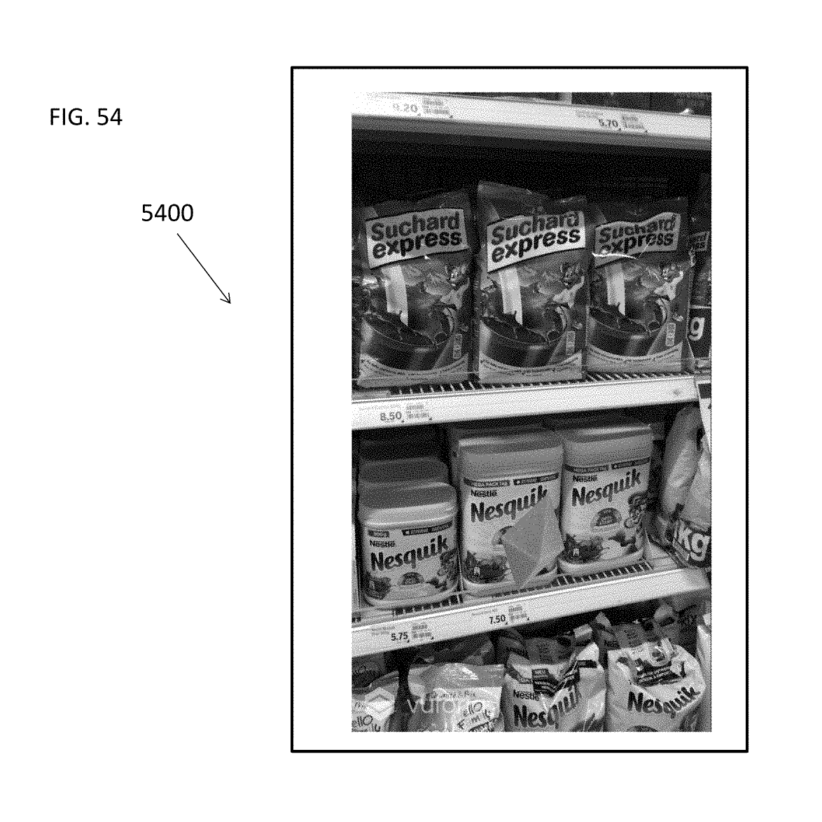

In one or more implementations, an augmented virtual reality mode is supported, which is particularly well suited for indoor use, such as at a retail establishment. In operation, one or more processors can be configured by executing code to detect that a player is near or inside a retail location. For example, GPS can be received and/or processed to determine a player's specific whereabouts. Thereafter, as a computing device associated with the player moves to a particular shelf/product, such as a predetermined shelf/product, a prompt can be provided on the computing device for the user to launch a camera configured with the computing device. Alternatively, the camera can be activated automatically. One or more images of the shelf/product (or other particular place or item) can be modified to include a gem 4004 or other relevant feature. The gem, thus, appears by means of augmented reality, and the player can interact with the app to collect the gem, just as described above with regard to collecting gems outdoors vis-à-vis a map. This has the added benefit of driving traffic to a respective location (e.g., a retailer) and/or to particular products, and does not require indoor navigation functionality or other complex technology. FIG. 54 illustrates an example image 5400 representing an item with a gem overlaid thereon, in accordance with an example implementation of the present application.

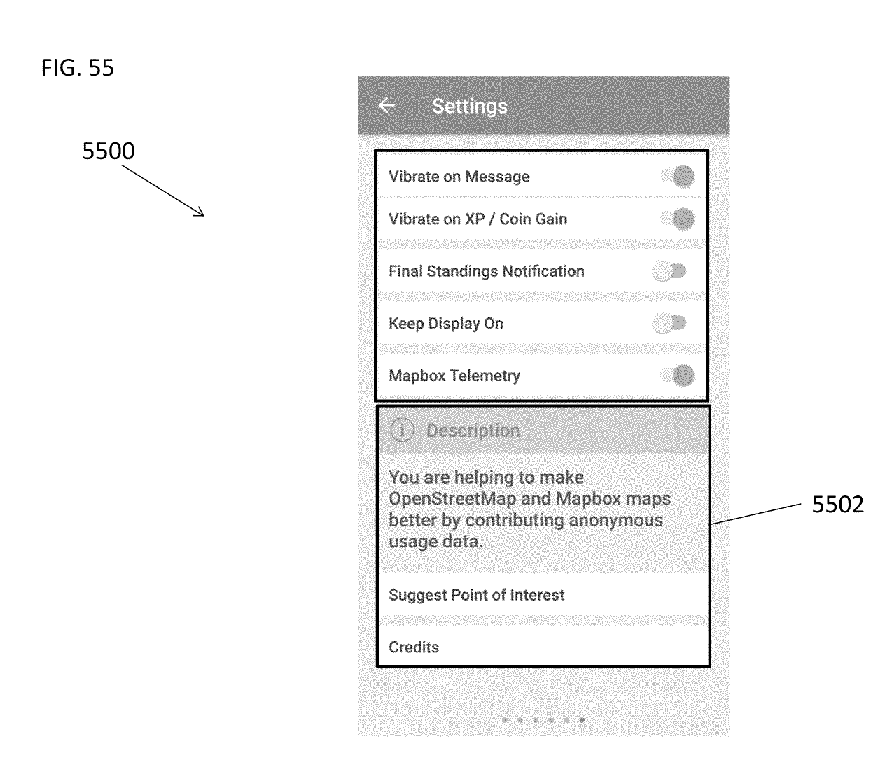

FIG. 55 illustrates an example display screen 5500 for defining respective user settings, in accordance with an example implementation of the present application. Included in display screen 5500 is crowd source section 5502, which enables users to suggest particular points of interest.

Thus, as shown and described herein, an integration of two pillars is provided: a health pillar that can include a walk score value and a health reservoir can be implemented and provided for value, such as reductions in insurance premiums. Further, a gaming pillar is the implemented or accessed, that can be driven by experience points and game levels, in which user interest is maintained by features such as quests, team battles and collection of items in mapped points of interest. Users can maintain and monitor good health practices, including to prevent decay of walk scores while simultaneously realizing value, such as in cost reductions.

Thus, as shown and described herein, an integration of two pillars is provided: a health pillar that can include a walk score value and a health reservoir can be implemented and provided for value, such as reductions in insurance premiums. Further, a gaming pillar is implemented or accessed, that can be driven by experience points and game levels, in which user interest is maintained by features such as quests, team battles and collection of items in mapped points of interest. Users can maintain and monitor good health practices, including to prevent decay of walk scores while simultaneously realizing value, such as in cost reductions.

The subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

The various embodiments described above disclose features that can optionally be combined in a variety of ways depending on the desired implementation. It will be appreciated that other embodiments based on different combinations of features are also possible. It will also be appreciated that more than one parameter for a particular parameter type can be used. None of the described features are mutually exclusive, and any combination of can be deployed to achieve the functions described above.

Thus, as shown and described herein, a plurality of interactive modules are provided to encourage individual users to submit information actively, as well as to track activity and health information passively. The information can be used for gaining key insights into the health of individuals, such as in connection with the calculation of a health score. In one or more implementations, pay as you live insurance options can be provided as a function of the health score information, and interactivity, such as via social Internet websites encourages users to promote health information and, perhaps more importantly, improve their health statues. Such functionality has been heretofore unavailable.

Although the present invention has been described in relation to particular embodiments thereof, many other variations and modifications and other uses will become apparent to those skilled in the art. It is preferred, therefore, that the present invention not be limited by the specific disclosure herein.

What is claimed:

1. A system for dynamic tracking of user-based activity as a function of information received from a plurality of data sources, the system comprising:
at least one computing device configured with a processor and non-transitory processor readable media and instructions that, when executed by the at least one computing device, configures the computing device to:
track via a first application user activity information correlated to steps taken by a user over a period of time, the first application executing on a mobile computing device and including a graphical user interface that presents at least a health score associated with the user;
communicate with a second application executed by the mobile computing device gameplay information, the communications including information concerning user interaction with a gaming environment provided by the mobile computing device;
combine, using the first application, the tracked user activity information with at least a portion of the user interaction information into an activity value;
update the health score of the user using the activity value;
store the updated health score in association with a record concerning the user; and
cause a notification on the mobile computing device of the updated health score when the first application is not executing.

2. A method for updating a health score of a user through dynamic tracking of user-based activity as a function of information received from a plurality of data sources, the method comprising:
tracking via a first application user activity information correlated to steps taken by the user over a period of time, the first application executing on a mobile computing device and including a graphical user interface that presents at least the health score;
communicating with a second application executed by the mobile computing device gameplay information, the communications including information concerning user interaction with a gaming environment provided by the mobile computing device;
combining, using the first application, the tracked user activity information with at least a portion of the user interaction information into an activity value;
updating the health score of the user using the activity value;
storing the updated health score in association with a record concerning the user; and
presenting the updated health score in the graphical user interface by causing a notification independent of launching the application.

3. The method of claim 2, wherein the health score is calculated, by the mobile computing device or a server computing device, in accordance with initial baseline information representing at least one of age, weight, and height of the user;

4. The method of claim 2, wherein the health score includes a walking score.

5. The method of claim 4, wherein the walking score represents an inverse of a reduction in all-cause mortality risk associated with walking.

6. The method of claim 4, wherein the walking score includes a buffer value that represents an amount of time that a user can rest before the walking score decays.

7. The method of claim 6, wherein the size of buffer depends on accumulated energy from successive footsteps taken by the user.

8. The method of claim 2, further comprising calculating by the mobile computing device a value representing a modulator risk based on risk for all-cause mortality associated with obesity.

9. The method of claim 2, further comprising calculating by the mobile computing device a value representing an energy amplifier from the obesity risk model to motivate overweight and obese users to walk.

10. The method of claim 2, further comprising calculating by the mobile computing device a value representing net years gained, which is a positive contribution from energy spent in walking, and a negative contribution from excess weight.

11. The method of claim 2, wherein the gaming environment provided by the mobile computing device includes at least one of: mapping functionality; game levels; social networking; and team-based activity.

12. The method of claim 2, further comprising reducing insurance premium as a function of the health score.

13. The method of claim 2, further comprising preventing double counting by accounting for user activity in the first application and the second application and eliminating one accounted user activity.

14. A method for dynamic tracking user-based activity as a function of information received from a plurality of data sources, the method comprising:
tracking via a first application user activity information correlated to steps taken by a user over a period of time, the first application executing on a mobile computing device and including a graphical user interface;
communicating with a second application executed by the mobile computing device gameplay information, the communications including information concerning user interaction with a gaming environment provided by the mobile computing device;
combining, using the first application, the tracked user activity information with at least a portion of the user interaction information into an activity value; and
automatically presenting the activity value in the graphical user interface of the first application when the first application is executing.
